(19) United States
(12) Patent Application Publication
(10) Pub. No.: US 2009/0036311 A1
(43) Pub. Date: Feb. 5, 2009

(54) Benzoyl-Substituted Alanines

(75) Inventors: Matthias Witschel, Bad Durkheim (DE); Cyrill Zagar, Hong Kong (CN); Eike Hupe, Ludwigshafen (DE); Toralf Kuhn, Mannheim (DE); William Karl Moberg, Hassloch (DE); Liliana Parra Rapado, Offenburg (DE); Frank Stelzer, Mannheim (DE); Andrea Vescovi, Mannheim (DE); Robert Reinhard, Limburgerhof (DE); Bernd Sievernich, Hassloch (DE); Klaus Grossmann, Neuhofen (DE); Thomas Ehrhardt, Speyer (DE)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/279,351

(22) PCT Filed: Feb. 7, 2007
(86) PCT No.: PCT/EP07/51158

§ 371 Date: Aug. 14, 2008

(30) Foreign Application Priority Data
Feb. 16, 2006 (EP) 06110013.7

(51) Int. Cl. A01N 37/18; C07C 237/34; C07C 229/34; A01N 37/44; A01P 13/00

(52) U.S. Cl. 504/315; 564/157; 564/153; 562/442; 560/37; 564/154; 504/322; 504/335

(57) ABSTRACT

The present invention relates to benzoyl-substituted alanines of the formula I

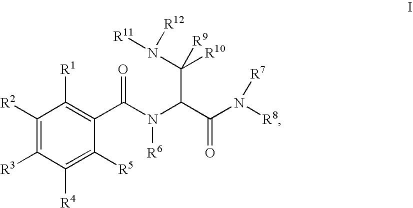

in which the variables R¹ to R¹² are as defined in the description,
and to their agriculturally useful salts,
to processes and intermediates for their preparation, and to the use of these compounds or of compositions comprising these compounds for controlling unwanted plants.

The present invention relates to benzoyl-substituted alanines of the formula I

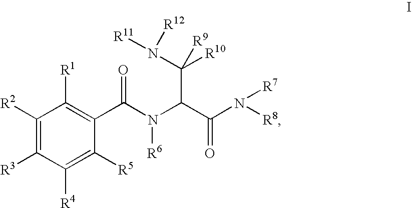

in which the variables are as defined below:
R¹ is halogen, cyano, C₁-C₆-alkyl, C₁-C₆-haloalkyl or C₁-C₆-haloalkoxy;
R², R³, R⁴, R⁵ are hydrogen, halogen, cyano, C₁-C₆-alkyl, C₁-C₆-haloalkyl, C₁-C₆-alkoxy or C₁-C₆-haloalkoxy;
R⁶, R⁷ are hydrogen, hydroxyl or C₁-C₆-alkoxy;
R⁸ is C₁-C₆-alkyl, C₁-C₄-cyanoalkyl or C₁-C₆-haloalkyl;
R⁹ is hydrogen or C₁-C₆-alkyl;
R¹⁰ is hydrogen, C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₁-C₆-cyanoalkyl, C₂-C₆-cyanoalkenyl, C₂-C₆-cyanoalkynyl, C₁-C₆-hydroxyalkyl, C₂-C₆-hydroxyalkenyl, C₂-C₆-hydroxyalkynyl, C₃-C₆-cycloalkyl, C₃-C₆-cycloalkenyl, 3- to 6-membered heterocyclyl,
   where the cycloalkyl, cycloalkenyl or 3- to 6-membered heterocyclyl radicals mentioned above may be partially or fully halogenated and/or may carry one to three radicals from the group consisting of oxo, cyano, nitro, C₁-C₆-alkyl, C₁-C₆-haloalkyl, hydroxyl, C₁-C₆-alkoxy, C₁-C₆-haloalkoxy, hydroxycarbonyl, C₁-C₆-alkoxycarbonyl, hydroxycarbonyl-C₁-C₆-alkoxy, C₁-C₆-alkoxycarbonyl-C₁-C₆-alkoxy, amino, C₁-C₆-alkylamino, di(C₁-C₆-alkyl)amino, C₁-C₆-alkylsulfonylamino, C₁-C₆-haloalkylsulfonylamino, aminocarbonylamino, (C₁-C₆-alkylamino)carbonylamino, di(C₁-C₆-alkyl)-aminocarbonylamino, aryl and aryl(C₁-C₆-alkyl);
   C₁-C₆-alkoxy-C₁-C₄-alkyl, C₂-C₆-alkenyloxy-C₁-C₄-alkyl, C₂-C₆-alkynyloxy-C₁-C₄-alkyl, C₁-C₆-haloalkoxy-C₁-C₄-alkyl, C₂-C₆-haloalkenyloxy-C₁-C₄-alkyl, C₂-C₆-haloalkynyloxy-C₁-C₄-alkyl, C₁-C₆-alkoxy-C₁-C₄-alkoxy-C₁-C₄-alkyl, C₁-C₆-alkylthio-C₁-C₄-alkyl, C₂-C₆-alkenylthio-C₁-C₄-alkyl, C₂-C₆-alkynylthio-C₁-C₄-alkyl, C₁-C₆-haloalkyl-C₁-C₄-thioalkyl, C₂-C₆-haloalkenyl-C₁-C₄-thioalkyl, C₂-C₆-haloalkynyl-C₁-C₄-thioalkyl, C₁-C₆-alkylsulfinyl-C₁-C₄-alkyl, C₁-C₆-haloalkylsulfinyl-C₁-C₄-alkyl, C₁-C₆-alkylsulfonyl-C₁-C₄-alkyl, C₁-C₆-haloalkylsulfonyl-C₁-C₄-alkyl, amino-C₁-C₄-alkyl, C₁-C₆-alkylamino-C₁-C₄-alkyl, di(C₁-C₆-alkyl)amino-C₁-C₄-alkyl, C₁-C₆-alkylsulfonylamino-C₁-C₄-alkyl, C₁-C₆-alkylsulfonyl(C₁-C₆-alkyl)amino-C₁-C₄-alkyl, C₁-C₆-alkylcarbonyl, hydroxycarbonyl, C₁-C₆-alkoxycarbonyl, aminocarbonyl, C₁-C₆-alkylaminocarbonyl, di(C₁-C₆-alkyl)aminocarbonyl, formylamino-C₁-C₄-alkyl, C₁-C₆-alkoxycarbonylamino-C₁-C₄-alkyl, C₁-C₆-alkylcarbonyl-C₁-C₆-alkyl, hydroxycarbonyl-C₁-C₄-alkyl, C₁-C₆-alkoxycarbonyl-C₁-C₄-alkyl, C₁-C₆-haloalkoxycarbonyl-C₁-C₄-alkyl, C₁-C₆-alkylcarbonyloxy-C₁-C₄-alkyl, aminocarbonyl-C₁-C₄-alkyl, C₁-C₆-alkylaminocarbonyl-C₁-C₄-alkyl, di(C₁-C₆-alkyl)aminocarbonyl-C₁-C₄-alkyl, C₁-C₆-alkylcarbonylamino-C₁-C₄-alkyl, C₁-C₆-alkylcarbonyl-(C₁-C₆-alkylamino)-C₁-C₄-alkyl, (C₁-C₆-alkyl)aminocarbonyloxy-C₁-C₄-alkyl, di(C₁-C₆-alkyl)aminocarbonyloxy-C₁-C₄-alkyl, [(C₁-C₆-alkyl)aminocarbonylamino]C₁-C₄-alkyl, [di(C₁-C₆-alkyl)aminocarbonylamino]C₁-C₄-alkyl;
   phenyl-C₁-C₄-alkyl, phenyl-C₂-C₄-alkenyl, phenyl-C₂-C₄-alkynyl, phenyl-C₁-C₄-haloalkyl, phenyl-C₂-C₄-haloalkenyl, phenyl-C₂-C₄-haloalkynyl, phenyl-C₁-C₄-hydroxyalkyl, phenyl-C₂-C₄-hydroxyalkenyl, phenyl-C₂-C₄-hydroxyalkynyl, phenylcarbonyl-C₁-C₄-alkyl, phenylcarbonyloxy-C₁-C₄-alkyl, phenyloxycarbonyl-C₁-C₄-alkyl, phenyloxy-C₁-C₄-alkyl, phenylthio-C₁-C₄-alkyl, phenylsulfinyl-C₁-C₄-alkyl, phenylsulfonyl-C₁-C₄-alkyl,
   heteroaryl, heteroaryl-C₁-C₄-alkyl, heteroaryl-C₂-C₄-alkenyl, heteroaryl-C₂-C₄-alkynyl, heteroaryl-C₁-C₄-haloalkyl, heteroaryl-C₂-C₄-haloalkenyl, heteroaryl-C₂-C₄-haloalkynyl, heteroaryl-C₁-C₄-hydroxyalkyl, heteroaryl-C₂-C₄-hydroxyalkenyl, heteroaryl-C₂-C₄-hydroxyalkynyl, heteroarylcarbonyl-C₁-C₄-alkyl, heteroarylcarbonyloxy-C₁-C₄-alkyl, heteroaryloxycarbonyl-C₁-C₄-alkyl, heteroaryloxy-C₁-C₄-alkyl, heteroarylthio-C₁-C₄-alkyl, heteroarylsulfinyl-C₁-C₄-alkyl, heteroarylsulfonyl-C₁-C₄-alkyl,
      where the phenyl and heteroaryl radicals mentioned above may be partially or fully halogenated and/or may carry one to three radicals from the group consisting of cyano, nitro, C₁-C₆-alkyl, C₁-C₆-haloalkyl, hydroxyl, C₁-C₆-hydroxyalkyl, C₁-C₆-alkoxy, C₁-C₆-haloalkoxy, hydroxycarbonyl, C₁-C₆-alkoxycarbonyl, hydroxycarbonyl-C₁-C₆-alkoxy, C₁-C₆-alkoxycarbonyl-C₁-C₆-alkoxy, amino, C₁-C₆-alkylamino, di(C₁-C₆-alkyl)amino, C₁-C₆-alkyl-sulfonylamino, C₁-C₆-haloalkylsulfonylamino, (C₁-C₆-alkylamino)-carbonylamino, di(C₁-C₆-alkyl)-aminocarbonylamino, aryl and aryl(C₁-C₆-alkyl);
R¹¹ is hydrogen, C₁-C₆-alkyl, C₃-C₆-cycloalkyl, C₃-C₆-alkenyl, C₃-C₆-alkynyl, C₃-C₆-haloalkenyl, C₃-C₆-haloalkynyl, formyl, C₁-C₆-alkylcarbonyl, C₃-C₆-cycloalkylcarbonyl, C₂-C₆-alkenylcarbonyl, C₂-C₆-alkynylcarbonyl, C₁-C₆-alkoxycarbonyl, C₃-C₆-alkenyloxycarbonyl, C₃-C₆-alkynyloxycarbonyl, aminocarbonyl, C₁-C₆-alkylaminocarbonyl, C₃-C₆-alkenylaminocarbonyl, C₃-C₆-alkynylaminocarbonyl, C₁-C₆-alkylsulfonylaminocarbonyl, di(C₁-C₆-alkyl)aminocarbonyl, N—(C₃-C₆-alkenyl)-N—(C₁-C₆-alkyl)aminocarbonyl, N—(C₃-C₆-alkynyl)-N—(C₁-C₆-alkyl)aminocarbonyl, N—(C₁-C₆-alkoxy)-N—(C₁-C₆-alkyl)aminocarbonyl, N—(C₃-C₆-alkenyl)-N—(C₁-C₆-alkoxy)aminocarbonyl, N—(C₃-C₆-alkynyl)-N—(C₁-C₆-alkoxy)aminocarbonyl, di(C₁-C₆-alkyl)aminothiocarbonyl, (C₁-C₆-alkyl)cyanoimino, (amino)cyanoimino, [(C₁-C₆-alkyl)amino]cyanoimino, di(C₁-C₆-alkyl)aminocyanoimino, C₁-C₆-alkylcarbonyl-C₁-C₆-alkyl, C₁-C₆-alkoxyimino-C₁-C₆-alkyl, N—(C₁-C₆-alkylamino)imino-C₁-C₆-alkyl, N-[di(C₁-C₆-alkyl)amino]imino-C₁-C₆-alkyl or tri-C₁-C₄-alkylsilyl,
   where the alkyl, cycloalkyl and alkoxy radicals mentioned may be partially or fully halogenated and/or may carry one to three of the following groups: cyano, hydroxyl, C₃-C₆-cycloalkyl, C₁-C₆-alkoxy-C₁-C₄-alkyl, C₁-C₄-alkoxy-C₁-C₄-alkoxy-C₁-C₄-alkyl, C₁-C₄-alkoxy, C₁-C₄-alkylthio, di(C₁-C₄-alkyl)amino, C₁-C₄-alkyl-C₁-C₆-alkoxycarbonylamino, C₁-C₄-alkylcarbonyl, hydroxycarbonyl, C₁-C₄-alkoxycarbonyl, aminocarbonyl, C₁-C₄-alkylaminocarbonyl, di(C₁-C₄-alkyl)aminocarbonyl or C₁-C₄-alkylcarbonyloxy;
   phenyl, phenyl-C₁-C₆-alkyl, phenylcarbonyl-C₁-C₆-alkyl, phenoxycarbonyl, phenylaminocarbonyl, phenylsulfonylaminocarbonyl, N—(C₁-C₆-alkyl)-N-(phenyl)aminocarbonyl, phenyl-C₁-C₆-alkylcarbonyl,
      where the phenyl radical may be partially or fully halogenated and/or may carry one to three of the following groups: nitro, cyano, C₁-C₄-alkyl, C₁-C₄-haloalkyl, C₁-C₄-alkoxy or C₁-C₄-haloalkoxy; or
   SO₂R¹³;
R¹² is hydrogen, C₁-C₆-alkyl, C₃-C₆-cycloalkyl, C₃-C₆-alkenyl, C₃-C₆-alkynyl, C₃-C₆-haloalkenyl, C₃-C₆-haloalkynyl, hydroxyl or C₁-C₆-alkoxy;
R¹³ is C₁-C₆-alkyl, C₁-C₆-haloalkyl or phenyl,
   where the phenyl radical may be partially or fully halogenated and/or may carry one to three of the following groups: C₁-C₆-alkyl, C₁-C₆-haloalkyl or C₁-C₆-alkoxy;
      or an agriculturally useful salt thereof.

Moreover, the invention relates to processes and intermediates for preparing compounds of the formula I, to compositions comprising them and to the use of these derivatives or of the compositions comprising them for controlling harmful plants.

2,ω-Diaminocarbonyl compounds with herbicidal activity are described, inter alia, in WO 03/045878.

Also known from the literature (for example WO 05/061464 and WO 05/061443) are heteroaroyl-substituted and benzoyl-substituted phenylalanines which may carry an optionally substituted amino group in the β-position.

However, the herbicidal properties of the prior-art compounds and/or their compatibility with crop plants are not entirely satisfactory.

Accordingly, it is an object of the present invention to provide novel, in particular herbicidally active, compounds having improved properties.

We have found that this object is achieved by the benzoyl-substituted alanines of the formula I and their herbicidal action.

Furthermore, we have found herbicidal compositions which comprise the compounds I and have very good herbicidal action. Moreover, we have found processes for preparing these compositions and methods for controlling unwanted vegetation using the compounds I.

Depending on the substitution pattern, the compounds of the formula I comprise two or more centers of chirality, in which case they are present as enantiomers or diastereomer mixtures. The invention provides both the pure enantiomers or diastereomers and their mixtures.

The compounds of the formula I may also be present in the form of their agriculturally useful salts, the nature of the salt generally being immaterial. Suitable salts are, in general, the cations or the acid addition salts of those acids whose cations and anions, respectively, have no adverse effect on the herbicidal action of the compounds I.

Suitable cations are in particular ions of the alkali metals, preferably lithium, sodium and potassium, of the alkaline earth metals, preferably calcium and magnesium, and of the transition metals, preferably manganese, copper, zinc and iron, and also ammonium, where, if desired, one to four hydrogen atoms may be replaced by C₁-C₄-alkyl, hydroxy-C₁-C₄-alkyl, C₁-C₄-alkoxy-C₁-C₄-alkyl, hydroxy-C₁-C₄-alkoxy-C₁-C₄-alkyl, phenyl or benzyl, preferably ammonium, dimethylammonium, diisopropylammonium, tetramethylammonium, tetrabutylammonium, 2-(2-hydroxyeth-1-oxy)eth-1-yl-ammonium, di-(2-hydroxyeth-1-yl)ammonium, trimethylbenzylammonium, furthermore phosphonium ions, sulfonium ions, preferably tri(C₁-C₄-alkyl)sulfonium, and sulfoxonium ions, preferably tri(C₁-C₄alkyl)sulfoxonium.

Anions of useful acid addition salts are primarily chloride, bromide, fluoride, hydrogensulfate, sulfate, dihydrogenphosphate, hydrogenphosphate, nitrate, bicarbonate, carbonate, hexafluorosilicate, hexafluorophosphate, benzoate, and the anions of C₁-C₄-alkanoic acids, preferably formate, acetate, propionate and butyrate.

The organic moieties mentioned for the substituents R¹-R¹³ or as radicals on phenyl, heterocyclyl, aryl, heteroaryl or heterocyclyl rings are collective terms for individual enumerations of the specific group members. All hydrocarbon chains, i.e. all alkyl, alkylsilyl, alkenyl, alkynyl, cyanoalkyl, haloalkyl, haloalkenyl, haloalkynyl, alkoxy, haloalkoxy, alkoxyalkyl, alkoxyalkoxyalkyl, alkylcarbonyl, alkenylcarbonyl, alkynylcarbonyl, alkoxycarbonyl, alkenyloxycarbonyl, alkynyloxycarbonyl, alkylamino, alkylsulfonylamino, haloalkylsulfonylamino, alkylalkoxycarbonylamino, alkylaminocarbonyl, alkenylaminocarbonyl, alkynylaminocarbonyl, alkylsulfonylaminocarbonyl, dialkylaminocarbonyl, N-alkenyl-N-alkylaminocarbonyl, N-alkynyl-N-alkylamino-carbonyl, N-alkoxy-N-alkylaminocarbonyl, N-alkenyl-N-alkoxyaminocarbonyl, N-alkynyl-N-alkoxyaminocarbonyl, dialkylaminothiocarbonyl, alkylcarbonylalkyl, alkoximinoalkyl, N-(alkylamino)iminoalkyl, N-(dialkylamino)iminoalkyl, formylamino-C₁-C₄-alkyl, C₁-C₆-alkoxycarbonylamino-C₁-C₄-alkyl, [(C₁-C₆-alkyl)amino-carbonylamino]C₁-C₄-alkyl, [di(C₁-C₆-alkyl)amino-carbonylamino]-C₁-C₄-alkyl, (C₁-C₆-alkyl)cyanoimino, [(C₁-C₆-alkyl)amino]cyanoimino, [di(C₁-C₆-alkyl)amino]cyanoimino, phenylalkyl, phenylcarbonylalkyl, N-alkyl-N-phenyl-aminocarbonyl, phenylalkylcarbonyl, arylalkyl, heterocyclylcarbonylalkyl, N-alkyl-N-heterocyclylaminocarbonyl, heterocyclylalkylcarbonyl, alkylthio and alkylcarbonyloxy moieties may be straight-chain or branched.

Unless indicated otherwise, halogenated substituents preferably carry one to five identical or different halogen atoms. The term halogen denotes in each case fluorine, chlorine, bromine or iodine.

Examples of other meanings are:
C₁-C₄-alkyl and also the alkyl moieties of tri-C₁-C₄-alkylsilyl, C₁-C₄-alkylcarbonyloxy, C₁-C₄-alkyl-C₁-C₄-alkoxycarbonylamino, C₁-C₆-alkyliminooxy-C₁-C₄-alkyl, C₁-C₄-alkoxy-C₁-C₄-alkoxy-C₁-C₄-alkyl, C₁-C₆-alkoxy-C₁-C₄-alkyl, C₂-C₆-alkenyloxy-C₁-C₄-alkyl, C₂-C₆-alkynyloxy-C₁-C₄-alkyl, C₁-C₆-haloalkoxy-C₁-C₄-alkyl, C₂-C₆-haloalkenyloxy-C₁-C₄-alkyl, C₂-C₆-haloalkynyloxy-C₁-C₄-alkyl, C₁-C₆-alkoxy-C₁-C₄-alkoxy-C₁-C₄-alkyl, C₁-C₆-alkylthio-C₁-C₄-alkyl, C₂-C₆-alkenylthio-C₁-C₄-alkyl, C₂-C₆-alkynylthio-C₁-C₄-alkyl, C₁-C₆-alkylsulfinyl-C₁-C₄-alkyl, C₁-C₆-haloalkylsulfinyl-C₁-C₄-alkyl, C₁-C₆-alkylsulfonyl-C₁-C₄-alkyl, C₁-C₆-haloalkylsulfonyl-C₁-C₄-alkyl, amino-C₁-C₄-alkyl, C₁-C₆-alkylamino-C₁-C₄-alkyl, di(C₁-C₆-alkyl)amino-C₁-C₄-alkyl, formylamino-C₁-C₄-alkyl, C₁-C₆-alkoxycarbonylamino-C₁-C₄-alkyl, C₁-C₆-alkyl-sulfonylamino-C₁-C₄-alkyl, C₁-C₆-alkylsulfonyl-(C₁-C₆-alkylamino)-C₁-C₄-alkyl, hydroxycarbonyl-C₁-C₄-alkyl, C₁-C₆-alkoxycarbonyl-C₁-C₄-alkyl, C₁-C₆-halo-alkoxycarbonyl-C₁-C₄-alkyl, C₁-C₆-alkylcarbonyloxy-C₁-C₄-alkyl, aminocarbonyl-C₁-C₄-alkyl, C₁-C₆-alkylaminocarbonyl-C₁-C₄-alkyl, di(C₁-C₆-alkyl)aminocarbonyl-C₁-C₄-alkyl, [(C₁-C₆-alkyl)aminocarbonylamino]C₁-C₄-alkyl, [di(C₁-C₆-alkyl)amino-carbonylamino]C₁-C₄-alkyl, C₁-C₆-alkylcarbonylamino-C₁-C₄-alkyl, C₁-C₆-alkylcarbonyl-(C₁-C₆-alkylamino)-C₁-C₄-alkyl, C₁-C₆-alkylaminocarbonyloxy-C₁-C₄-alkyl, [di(C₁-C₆-alkylamino)carbonyloxy]C₁-C₄-alkyl, phenyl-C₁-C₄-alkyl, heteroarylcarbonyl-C₁-C₄-alkyl, heteroarylcarbonyloxy-C₁-C₄-alkyl, heteroaryloxycarbonyl-C₁-C₄-alkyl, heteroaryloxy-C₁-C₄-alkyl, heteroarylthio-C₁-C₄-alkyl, heteroarylsulfinyl-C₁-C₄-alkyl, heteroarylsulfonyl-C₁-C₄-alkyl, and aryl(C₁-C₄-alkyl):
for example methyl, ethyl, n-propyl, 1-methylethyl, n-butyl, 1-methylpropyl, 2-methylpropyl and 1,1-dimethylethyl;
C₁-C₆-alkyl and also the alkyl moieties of C₁-C₆-cyanoalkyl, C₁-C₆-alkoxycarbonyl-C₁-C₆-alkyl, C₁-C₆-alkylsulfonylamino, C₁-C₆-alkylsulfonylaminocarbonyl, N—(C₃-C₆-alkenyl)-N—(C₁-C₆-alkyl)aminocarbonyl, (C₃-C₆-alkynyl)-N—(C₁-C₆-alkyl)-aminocarbonyl, N—(C₁-C₆-alkoxy)-N—(C₁-C₆-alkyl)aminocarbonyl, C₁-C₆-alkylcarbonyl-C₁-C₆-alkyl, C₁-C₆-alkoxyimino-C₁-C₆-alkyl, N—(C₁-C₆-alkylamino)imino-C₁-C₆-alkyl, N-(di-C₁-C₆-alkylamino)imino-C₁-C₆-alkyl, (C₁-C₆-alkyl)cyanoimino, phenyl-C₁-C₆-alkyl, phenylcarbonyl-C₁-C₆-alkyl, N—(C₁-C₆-alkyl)-N-phenylaminocarbonyl, heterocyclyl-C₁-C₆-alkyl, heterocyclylcarbonyl-C₁-C₆-alkyl and N—(C₁-C₆-alkyl)-N-heterocyclylaminocarbonyl:
C₁-C₄-alkyl as mentioned above, and also, for example, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methyl-pentyl, 4-methylpentyl, 1,1-dimethylbutyl, 1,2-dimethylbutyl, 1,3-di-methylbutyl, 2,2-dimethylbutyl, 2,3-dimethylbutyl, 3,3-dimethylbutyl, 1-ethylbutyl, 2-ethylbutyl, 1,1,2-trimethylpropyl, 1-ethyl-1-methylpropyl and 1-ethyl-3-methylpropyl;
C₁-C₄-alkylcarbonyl: for example methylcarbonyl, ethylcarbonyl, propylcarbonyl, 1-methylethylcarbonyl, butylcarbonyl, 1-methylpropylcarbonyl, 2-methylpropyl-carbonyl or 1,1-dimethylethylcarbonyl;
C₁-C₆-alkylcarbonyl and also the alkylcarbonyl radicals of C₁-C₆-alkylcarbonyl-C₁-C₆-alkyl, C₁-C₆-alkylcarbonyloxy-C₁-C₆-alkyl, C₁-C₆-alkylcarbonylamino-C₁-C₄-alkyl, phenyl-C₁-C₆-alkylcarbonyl and heterocyclyl-C₁-C₆-alkylcarbonyl, C₁-C₆-alkylcarbonyl-(C₁-C₆-alkylamino)-C₁-C₄-alkyl:
C₁-C₄-alkylcarbonyl as mentioned above, and also, for example, pentylcarbonyl, 1-methylbutylcarbonyl, 2-methylbutylcarbonyl, 3-methylbutylcarbonyl, 2,2-dimethylpropylcarbonyl, 1-ethylpropylcarbonyl, hexylcarbonyl, 1,1-dimethylpropylcarbonyl, 1,2-dimethylpropylcarbonyl, 1-methylpentylcarbonyl, 2-methylpentylcarbonyl, 3-methylpentylcarbonyl, 4-methylpentylcarbonyl, 1,1-dimethylbutylcarbonyl, 1,2-dimethylbutylcarbonyl, 1,3-dimethylbutylcarbonyl, 2,2-dimethylbutylcarbonyl, 2,3-dimethylbutylcarbonyl, 3,3-dimethylbutylcarbonyl, 1-ethylbutylcarbonyl, 2-ethylbutylcarbonyl, 1,1,2-trimethylpropylcarbonyl, 1,2,2-trimethylpropylcarbonyl, 1-ethyl-1-methylpropylcarbonyl or 1-ethyl-2-methylpropylcarbonyl;
C₃-C₆-cycloalkyl and also the cycloalkyl moieties of C₃-C₆-cycloalkylcarbonyl: monocyclic saturated hydrocarbons having 3 to 6 ring members, such as cyclopropyl, cyclobutyl, cyclopentyl and cyclohexyl;
C₃-C₆-cycloalkenyl: for example 1-cyclopropenyl, 2-cyclopropenyl, 1-cyclobutenyl, 2-cyclobutenyl, 1-cyclopentenyl, 2-cyclopentenyl, 1,3-cyclopentadienyl, 1,4-cyclopentadienyl, 2,4-cyclopentadienyl, 1-cyclohexenyl, 2-cyclohexenyl, 3-cyclohexenyl, 1,3-cyclohexadienyl, 1,4-cyclohexadienyl, 2,5-cyclohexadienyl;
C₃-C₆-alkenyl and also the alkenyl moieties of C₃-C₆-alkenyloxycarbonyl, C₃-C₆-alkenylaminocarbonyl, N—(C₃-C₆-alkenyl)-N—(C₁-C₆-alkyl)aminocarbonyl and N—(C₃-C₆-alkenyl)-N—(C₁-C₆-alkoxy)aminocarbonyl: for example 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
C₂-C₆-alkenyl and also the alkenyl moieties of C₂-C₆-alkenylcarbonyl, C₂-C₆-alkenyloxy-C₁-C₄-alkyl, C₂-C₆-alkenylthio-C₁-C₄-alkyl, phenyl-C₂-C₄-alkenyl, heteroary-C₂-C₄-alkenyl: C₃-C₆-alkenyl as mentioned above, and also ethenyl;
C₃-C₆-alkynyl and also the alkynyl moieties of C₃-C₆-alkynyloxycarbonyl, C₃-C₆-alkynylaminocarbonyl, N—(C₃-C₆-alkynyl)-N—(C₁-C₆-alkyl)aminocarbonyl, N—(C₃-C₆-alkynyl)-N—(C₁-C₆-alkoxy)aminocarbonyl: for example 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
C₂-C₆-alkynyl and also the alkynyl moieties of C₂-C₆-alkynylcarbonyl, C₂-C₂-alkynyloxy-C₁-C₄-alkyl, C₂-C₆-alkynylthio-C₁-C₄-alkyl, phenyl-C₂-C₄-alkynyl, heteroaryl-C₂-C₄-alkynyl: C₃-C₆-alkynyl as mentioned above, and also ethynyl;
C₁-C₄-cyanoalkyl: for example cyanomethyl, 1-cyanoeth-1-yl, 2-cyanoeth-1-yl, 1-cyanoprop-1-yl, 2-cyanoprop-1-yl, 3-cyanoprop-1-yl, 1-cyanoprop-2-yl, 2-cyano-prop-2-yl, 1-cyanobut-1-yl, 2-cyanobut-1-yl, 3-cyanobut-1-yl, 4-cyanobut-1-yl, 1-cyanobut-2-yl, 2-cyanobut-2-yl, 1-cyanobut-3-yl, 2-cyanobut-3-yl, 1-cyano-2-methylprop-3-yl, 2-cyano-2-methylprop-3-yl, 3-cyano-2-methylprop-3-yl and 2-cyanamethylprop-2-yl;
C₁-C₄-hydroxyalkyl and also the C₁-C₄-hydroxyalkyl moieties of phenyl-C₁-C₄-hydroxyalkyl, heteroaryl-C₁-C₄-hydroxyalkyl: for example hydroxymethyl, 1-hydroxyeth-1-yl, 2-hydroxyeth-1-yl, 1-hydroxyprop-1-yl, 2-hydroxyprop-1-yl, 3-hydroxyprop-1-yl, 1-hydroxyprop-2-yl, 2-hydroxyprop-2-yl, 1-hydroxybut-1-yl, 2-hydroxybut-1-yl, 3-hydroxybut-1-yl, 4-hydroxybut-1-yl, 1-hydroxybut-2-yl, 2-hydroxybut-2-yl, 1-hydroxybut-3-yl, 2-hydroxybut-3-yl, 1-hydroxy-2-methylprop-3-yl, 2-hydroxy-2-methylprop-3-yl, 3-hydroxy-2-methylprop-3-yl and 2-hydroxymethyl-prop-2-yl, 1,2-dihydroxyethyl, 1,2-dihydroxyprop-3-yl, 2,3-dihydroxyprop-3-yl, 1,2-dihydroxyprop-2-yl, 1,2-dihydroxybut-4-yl, 2,3-dihydroxybut-4-yl, 3,4-dihydroxybut-4-yl, 1,2-dihydroxybut-2-yl, 1,2-dihydroxybut-3-yl, 2,3-dihydroxybut-3-yl, 1,2-dihydroxy-2-methylprop-3-yl, 2,3-dihydroxy-2-methylprop-3-yl;
C₁-C₆-hydroxyalkyl: C₁-C₄-hydroxyalkyl as mentioned above and also, for example, 1-hydroxypent-5-yl, 2-hydroxypent-5-yl, 3-hydroxypent-5-yl, 4-hydroxypent-5-yl, 5-hydroxypent-5-yl, 1-hydroxypent-4-yl, 2-hydroxypent-4-yl, 3-hydroxypent-4-yl, 4-hydroxypent-4-yl, 1-hydroxypent-3-yl, 2-hydroxypent-3-yl, 3-hydroxypent-3-yl, 1-hydroxy-2-methylbut-3-yl, 2-hydroxy-2-methylbut-3-yl, 3-hydroxy-2-methylbut-3-yl, 1-hydroxy-2-methylbut-4-yl, 2-hydroxy-2-methylbut-4-yl, 3-hydroxy-2-methylbut-4-yl, 4-hydroxy-2-methylbut-4-yl, 1-hydroxy-3-methylbut-4-yl, 2-hydroxy-3-methylbut-4-yl, 3-hydroxy-3-methylbut-4-yl, 4-hydroxy-3-methylbut-4-yl, 1-hydroxyhex-6-yl, 2-hydroxyhex-6-yl, 3-hydroxyhex-6-yl, 4-hydroxyhex-6-yl, 5-hydroxyhex-6-yl, 6-hydroxyhex-6-yl, 1-hydroxy-2-methylpent-5-yl, 2-hydroxy-2-methylpent-5-yl, 3-hydroxy-2-methylpent-5-yl, 4-hydroxy-2-methylpent-5-yl, 5-hydroxy-2-methylpent-5-yl, 1-hydroxy-3-methylpent-5-yl, 2-hydroxy-3-methylpent-5-yl, 3-hydroxy-3-methyl-pent-5-yl, 4-hydroxy-3-methylpent-5-yl, 5-hydroxy-3-methylpent-5-yl, 1-hydroxy-4-methylpent-5-yl, 2-hydroxy-4-methylpent-5-yl, 3-hydroxy-4-methylpent-5-yl, 4-hydroxy-4-methylpent-5-yl, 5-hydroxy-4-methylpent-5-yl, 1-hydroxy-5-methylpent-5-yl, 2-hydroxy-5-methylpent-5-yl, 3-hydroxy-5-methylpent-5-yl, 4-hydroxy-5-methylpent-5-yl, 5-hydroxy-5-methylpent-5-yl, 1-hydroxy-2,3-dimethylbut-4-yl, 2-hydroxy-2,3-dimethylbut-4-yl, 3-hydroxy-2,3-dimethylbut-4-yl, 4-hydroxy-2,3-dimethylbut-4-yl, 1,2-dihydroxy-pent-5-yl, 2,3-dihydroxy-pent-5-yl, 3,4-dihydroxy-pent-5-yl, 4,5-dihydroxy-pent-5-yl, 1,2-dihydroxypent-4-yl, 2,3-dihydroxypent-4-yl, 3,4-dihydroxypent-4-yl, 4,5-dihydroxypent-4-yl, 1,2-dihydroxypent-3-yl, 2,3-dihydroxypent-3-yl, 1,2-dihydroxy-2-methylbut-3-yl, 2,3-dihydroxy-2-methylbut-3-yl, 3,4-dihydroxy-2-methylbut-3-yl, 2-hydroxy-2-hydroxymethylbut-3-yl, 1,2-dihydroxy-2-methylbut-4-yl, 2,3-dihydroxy-2-methylbut-4-yl, 3,4-dihydroxy-2-methylbut-4-yl, 1,2-dihydroxy-3-methylbut-4-yl, 2,3-dihydroxy-3-methylbut-4-yl, 3,4-dihydroxy-3-methylbut-4-yl, 3-hydroxy-3-hydroxymethylbut-4-yl, 1,2-dihydroxyhex-6-yl, 2,3-dihydroxy-hex-6-yl, 3,4-dihydroxyhex-6-yl, 4,5-dihydroxyhex-6-yl, 5,6-dihydroxyhex-6-yl, 1,2-dihydroxy-2-methylpent-5-yl, 2,3-dihydroxy-2-methylpent-5-yl, 3,4-dihydroxy-2-methylpent-5-yl, 4,5-dihydroxy-2-methylpent-5-yl, 2-hydroxy-2-hydroxymethylpent-5-yl, 1,2-dihydroxy-3-methylpent-5-yl, 2,3-dihydroxy-3-methylpent-5-yl, 3,4-dihydroxy-3-methylpent-5-yl, 4,5-dihydroxy-3-methylpent-5-yl, 3-hydroxy-3-hydroxymethylpent-5-yl, 1,2-dihydroxy-4-methylpent-5-yl, 2,3-dihydroxy-4-methylpent-5-yl, 3,4-dihydroxy-4-methylpent-5-yl, 4,5-dihydroxy-4-methylpent-5-yl, 4-hydroxy-4-hydroxymethylpent-5-yl, 1,2-dihydroxy-5-methylpent-5-yl, 2,3-dihydroxy-5-methylpent-5-yl, 3,4-dihydroxy-5-methylpent-5-yl, 4,5-dihydroxy-5-methylpent-5-yl, 5-hydroxy-5-hydroxymethylpent-5-yl, 1,2-dihydroxy-2,3-dimethylbut-4-yl, 2,3-dihydroxy-2,3-dimethylbut-4-yl, 3,4-dihydroxy-2,3-dimethylbut-4-yl, 2-hydroxy-2-hydroxymethyl-3-methylbut-4-yl, 3-hydroxy-3-hydroxymethyl-2-methylbut-4-yl;
C₁-C₄-haloalkyl and also the haloalkyl moieties of phenyl-C₁-C₄-haloalkyl, heteroaryl-C₁-C₄-haloalkyl: a C₁-C₄-alkyl radical as mentioned above which is partially or fully substituted by fluorine, chlorine, bromine and/or iodine, i.e., for example, chloromethyl, dichloromethyl, trichloromethyl, fluoromethyl, difluoromethyl, trifluoromethyl, chlorofluoromethyl, dichlorofluoromethyl, chlorodifluoromethyl, bromomethyl, iodomethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoro-ethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl, nonafluorobutyl, 1,1,2,2-tetrafluoroethyl and 1-trifluoromethyl-1,2,2,2,2-tetrafluoroethyl;
C₁-C₆-haloalkyl and also the haloalkyl moieties of C₁-C₆-haloalkylsulfonylamino, C₁-C₆-haloalkyl-C₁-C₄-thioalkyl: C₁-C₄-haloalkyl as mentioned above, and also, for example, 5-fluoropentyl, 5-chloropentyl, 5-bromopentyl, 5-iodopentyl, undecafluoropentyl, 6-fluorohexyl, 6-chlorohexyl, 6-bromohexyl, 6-iodohexyl and tridecafluorohexyl;
C₃-C₆-haloalkenyl: a C₃-C₆-alkenyl radical as mentioned above which is partially or fully substituted by fluorine, chlorine, bromine and/or iodine, for example 2-chloro-prop-2-en-1-yl, 3-chloroprop-2-en-1-yl, 2,3-dichloroprop-2-en-1-yl, 3,3-dichloroprop-2-en-1-yl, 2,3,3-trichloro-2-en-1-yl, 2,3-dichlorobut-2-en-1-yl, 2-bromoprop-2-en-1-yl, 3-bromoprop-2-en-1-yl, 2,3-dibromoprop-2-en-1-yl, 3,3-dibromoprop-2-en-1-yl, 2,3,3-tribromo-2-en-1-yl or 2,3-dibromobut-2-en-1-yl;
C₂-C₆-haloalkenyl and also the C₂-C₆-haloalkenyl moieties of C₂-C₆-haloalkenyloxy-C₁-C₄-alkyl, C₂-C₆-haloalkenyl-C₁-C₄-thioalkyl, phenyl-C₂-C₄-haloalkenyl, heteroaryl-C₂-C₄-haloalkenyl: a C₂-C₆-alkenyl radical as mentioned above which is partially or fully substituted by fluorine, chlorine, bromine and/or iodine: for example 2-chloro-vinyl, 2-chloroallyl, 3-chloroallyl, 2,3-dichloroallyl, 3,3-dichloroallyl, 2,3,3-trichloroallyl, 2,3-dichlorobut-2-enyl, 2-bromovinyl, 2-bromoallyl, 3-bromoallyl, 2,3-dibromoallyl, 3,3-dibromoallyl, 2,3,3-tribromoallyl or 2,3-dibromobut-2-enyl;
C₂-C₆— cyanoalkenyl: for example 2-cyanovinyl, 2-cyanoallyl, 3-cyanoallyl, 2,3-dicyanoallyl, 3,3-dicyanoallyl, 2,3,3-tricyanoallyl, 2,3-dicyanobut-2-enyl;
C₂-C₆-hydroxyalkenyl and also the hydroxyl moieties of phenyl-C₁-C₄-hydroxyalkenyl, heteroaryl-C₁-C₄-hydroxyalkenyl: for example 2-hydroxyvinyl, 2-hydroxyallyl, 3-hydroxyallyl, 2,3-dihydroxyallyl, 3,3-dihydroxyallyl, 2,3,3-trihydroxyallyl, 2,3-dihydroxybut-2-enyl;
C₃-C₆-haloalkynyl: a C₃-C₆-alkynyl radical as mentioned above which is partially or fully substituted by fluorine, chlorine, bromine and/or iodine, for example 1,1-difluoroprop-2-yn-1-yl, 3-iodoprop-2-yn-1-yl, 4-fluorobut-2-yn-1-yl, 4-chlorobut-2-yn-1-yl, 1,1-difluorobut-2-yn-1-yl, 4-iodobut-3-yn-1-yl, 5-fluoropent-3-yn-1-yl, 5-iodopent-4-yn-1-yl, 6-fluorohex-4-yn-1-yl or 6-iodohex-5-yn-1-yl;
C₂-C₆-haloalkynyl and also the C₂-C₆-haloalkynyl moieties of C₂-C₆-haloalkynyloxy-C₁-C₄-alkyl, C₂-C₆-haloalkynyl-C₁-C₄-thioalkyl, phenyl-C₂-C₄-haloalkynyl, heteroaryl-C₂-C₄-haloalkynyl: a C₂-C₆-alkynyl radical as mentioned above which is partially or fully substituted by fluorine, chlorine, bromine and/or iodine, for example 1,1-di-fluoroprop-2-yn-1-yl, 3-iodoprop-2-yn-1-yl, 4-fluorobut-2-yn-1-yl, 4-chlorobut-2-yn-1-yl, 1,1-difluorobut-2-yn-1-yl, 4-iodobut-3-yn-1-yl, 5-fluoropent-3-yn-1-yl, 5-iodopent-4-yn-1-yl, 6-fluorohex-4-yn-1-yl or 6-iodohex-5-yn-1-yl;
C₂-C₆-cyanoalkynyl: for example 1,1-dicyanoprop-2-yn-1-yl, 3-cyanoprop-2-yn-1-yl, 4-cyano-but-2-yn-1-yl, 1,1-dicyanobut-2-yn-1-yl, 4-cyanobut-3-yn-1-yl, 5-cyanopent-3-yn-1-yl, 5-cyanopent-4-yn-1-yl, 6-cyanohex-4-yn-1-yl or 6-cyanohex-5-yn-1-yl;
C₂-C₆-hydroxyalkynyl and also the hydroxyl moieties of phenyl-C₂-C₄-hydroxyalkynyl: for example 1,1-dihydroxyprop-2-yn-1-yl, 3-hydroxyprop-2-yn-1-yl, 4-hydroxybut-2-yn-1-yl, 1,1-dihydroxybut-2-yn-1-yl, 4-hydroxybut-3-yn-1-yl, 5-hydroxypent-3-yn-1-yl, 5-hydroxypent-4-yn-1-yl, 6-hydroxyhex-4-yn-1-yl or 6-hydroxyhex-5-yn-1-yl;
C₁-C₆alkylsulfinyl(C₁-C₆-alkyl-S(═O)—) and also the C₁-C₆-alkylsulfinyl moieties of C₁-C₆-alkylsulfinyl-C₁-C₄-alkyl: for example methylsulfinyl, ethylsulfinyl, propylsulfinyl, 1-methylethylsulfinyl, butylsulfinyl, 1-methylpropylsulfinyl, 2-methylpropylsulfinyl, 1,1-dimethylethylsulfinyl, pentylsulfinyl, 1-methylbutylsulfinyl, 2-methylbutylsulfinyl, 3-methylbutylsulfinyl, 2,2-cimethylpropylsulfinyl, 1-ethylpropylsulfinyl, 1,1-dimethylpropylsulfinyl, 1,2-dimethylpropylsulfinyl, hexyl-sulfinyl, 1-methylpentylsulfinyl, 2-methylpentylsulfinyl, 3-methylpentylsulfinyl, 4 methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethyl-propylsulfinyl, 1,2,2-trimethylpropylsulfinyl, 1-ethyl-1-methylpropylsulfinyl and 1-ethyl-2-methylpropylsulfinyl;
C₁-C₆-haloalkylsulfinyl and also the C₁-C₆-haloalkylsulfinyl moieties of C₁-C₆-haloalkylsulfinyl-C₁-C₄-alkyl: C₁-C₆-alkylsulfinyl radical as mentioned above which is partially or fully substituted by fluorine, chlorine, bromine and/or iodine, i.e. for example fluoromethylsulfinyl, difluoromethylsulfinyl, trifluoromethylsulfinyl, chlorodifluoromethylsulfinyl, bromodifluoromethylsulfinyl, 2-fluoroethylsulfinyl, 2-chloroethylsulfinyl, 2-bromoethylsulfinyl, 2-iodoethylsulfinyl, 2,2-difluoroethylsulfinyl, 2,2,2-trifluoroethylsulfinyl, 2,2,2-trichloroethylsulfinyl, 2-chloro-2-fluoroethylsulfinyl, 2-chloro-2,2-difluoroethylsulfinyl, 2,2-dichloro-2-fluoroethylsulfinyl, pentafluoroethylsulfinyl, 2-fluoropropylsulfinyl, 3-fluoropropylsulfinyl, 2-chloropropylsulfinyl, 3-chloropropylsulfinyl, 2-bromopropylsulfinyl, 3-bromopropylsulfinyl, 2,2-difluoropropylsulfinyl, 2,3-difluoro-propylsulfinyl, 2,3-dichloropropylsulfinyl, 3,3,3-trifluoropropylsulfinyl, 3,3,3-trichloro-propylsulfinyl, 2,2,3,3,3-pentafluoropropylsulfinyl, heptafluoropropylsulfinyl, 1-(fluoromethyl)-2-fluoroethylsulfinyl, 1-(chloromethyl)-2-chloroethylsulfinyl, 1-(bromomethyl)-2-bromoethylsulfinyl, 4-fluorobutylsulfinyl, 4-chlorobutylsulfinyl, 4 bromobutylsulfinyl, nonafluorobutylsulfinyl, 5-fluoropentylsulfinyl, 5-chloropentyl-sulfinyl, 5-bromopentylsulfinyl, 5-iodopentylsulfinyl, undecafluoropentylsulfinyl, 6-fluorohexylsulfinyl, 6-chlorohexylsulfinyl, 6-bromohexylsulfinyl, 6-iodohexylsulfinyl and tridecafluorohexylsulfinyl;
C₁-C₆-alkylsulfonyl(C₁-C₆-alkyl-S(O)₂) and also the C₁-C₆-alkylsulfonyl moieties of C₁-C₆-alkylsulfonyl-C₁-C₄-alkyl, C₁-C₆-alkylsulfonylamino, C₁-C₆-alkylsulfonylamino-C₁-C₄-alkyl, C₁-C₆-alkylsulfonyl-(C₁-C₆-alkylamino)-C₁-C₄-alkyl: for example methylsulfonyl, ethylsulfonyl, propylsulfonyl, 1-methylethylsulfonyl, butylsulfonyl, 1-methylpropylsulfonyl, 2-methylpropylsulfonyl, 1,1-dimethylethylsulfonyl, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl and 1-ethyl-2-methylpropylsulfonyl;
C₁-C₆-haloalkylsulfonyl and also the C₁-C₆-aloalkylsulfonyl moieties of C₁-C₆-haloalkylsulfonyl-C₁-C₄-alkyl, C₁-C₆-haloalkylsulfonylamino: a C₁-C₆-alkylsulfonyl radical as mentioned above which is partially or fully substituted by fluorine, chlorine, bromine and/or iodine, i.e. for example fluoromethylsulfonyl, difluoromethylsulfonyl, trifluoromethylsulfonyl, chlorodifluoromethylsulfonyl, bromodifluoromethylsulfonyl, 2-fluoroethylsulfonyl, 2-chloroethylsulfonyl, 2-bromoethylsulfonyl, 2-iodoethylsulfonyl, 2,2-difluoroethyl-sulfonyl, 2,2,2-trifluoroethylsulfonyl, 2-chloro-2-fluoroethylsulfonyl, 2-chloro-2,2-difluoroethylsulfonyl, 2,2-dichloro-2-fluoroethylsulfonyl, 2,2,2-trichloroethylsulfonyl, pentafluoroethylsulfonyl, 2-fluoropropylsulfonyl, 3-fluoropropylsulfonyl, 2-chloropropylsulfonyl, 3-chloropropylsulfonyl, 2-bromopropylsulfonyl, 3-bromopropylsulfonyl, 2,2-difluoropropylsulfonyl, 2,3-difluoropropylsulfonyl, 2,3-dichloropropylsulfonyl, 3,3,3-trifluoropropylsulfonyl, 3,3,3-trichloropropylsulfonyl, 2,2,3,3,3-pentafluoropropylsulfonyl, heptafluoropropylsulfonyl, 1-(fluoromethyl)-2-fluoroethylsulfonyl, 1-(chloromethyl)-2-chloroethylsulfonyl, 1-(bromomethyl)-2-bromoethylsulfonyl, 4-fluorobutylsulfonyl, 4-chlorobutylsulfonyl, 4-bromobutylsulfonyl, nonafluorobutylsulfonyl, 5-fluoropentylsulfonyl, 5-chloropentylsulfonyl, 5-bromopentylsulfonyl, 5-iodopentylsulfonyl, 6 fluorohexylsulfonyl, 6-bromohexylsulfonyl, 6-Iodohexylsulfonyl and tridecafluorohexylsulfonyl;
C₁-C₄-alkoxy and also the alkoxy moieties of hydroxycarbonyl-C₁-C₄-alkoxy, C₁-C₄-alkoxycarbonyl-C₁-C₄-alkoxy, C₁-C₄-alkoxy-C₁-C₄-alkoxy-C₁-C₄-alkyl and C₁-C₄-alkyl-C₁-C₄-alkoxycarbonylamino: for example methoxy, ethoxy, propoxy, 1-methylethoxy, butoxy, 1-methylpropoxy, 2-methylpropoxy and 1,1-dimethylethoxy;
C₁-C₆-alkoxy and also the alkoxy moieties of hydroxycarbonyl-C₁-C₆-alkoxy, C₁-C₆-alkoxycarbonyl-C₁-C₆-alkoxy, N—(C₁-C₆-alkoxy)-N—(C₁-C₆-alkyl)aminocarbonyl, N—(C₃-C₆-alkenyl)-N—(C₁-C₆-alkoxy)aminocarbonyl, N—(C₃-C₆-alkynyl)-N—(C₁-C₆-alkoxy)aminocarbonyl and C₁-C₆-alkoxyimino-C₁-C₆-alkyl: C₁-C₄-alkoxy as mentioned above, and also, for example, pentoxy, 1-methylbutoxy, 2-methylbutoxy, 3-methoxylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methyl-propoxy and 1-ethyl-2-methylpropoxy;
C₁-C₄-haloalkoxy: a C₁-C₄-alkoxy radical as mentioned above which is partially or fully substituted by fluorine, chlorine, bromine and/or iodine, i.e., for example, fluoromethoxy, difluoromethoxy, trifluoromethoxy, chlorodifluoromethoxy, bromo-difluoromethoxy, 2-fluoroethoxy, 2-chloroethoxy, 2-bromomethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoro-ethoxy, 2-fluoropropoxy, 3-fluoropropoxy, 2-chloropropoxy, 3-chloropropoxy, 2-bromopropoxy, 3-bromopropoxy, 2,2-difluoropropoxy, 2,3-difluoropropoxy, 2,3-dichloropropoxy, 3,3,3-trifluoropropoxy, 3,3,3-trichloropropoxy, 2,2,3,3,3-penta-fluoropropoxy, heptafluoropropoxy, 1-(fluoromethyl)-2-fluoroethoxy, 1-(chloromethyl)-2-chloroethoxy, 1-(bromomethyl)-2-bromoethoxy, 4-fluorobutoxy, 4-chlorobutoxy, 4-bromobutoxy and nonafluorobutoxy;
C₁-C₆-haloalkoxy and also the C₁-C₆-haloalkoxy moieties of C₁-C₆-haloalkoxy-C₁-C₄-alkyl, C₁-C₆-haloalkoxycarbonyl-C₁-C₄-alkyl: C₁-C₄-haloalkoxy as mentioned above, and also, for example, 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy and tridecafluorohexoxy;
C₁-C₆-alkoxy-C₁-C₄-alkyl and also the C₁-C₆-alkoxy-C₁-C₄-alkyl moieties of C₁-C₆-alkoxy-C₁-C₄-alkoxy-C₁-C₄-alkyl: C₁-C₄-alkyl which is substituted by C₁-C₆-alkoxy as mentioned above, i.e., for example, methoxymethyl, ethoxymethyl, propoxymethyl, (1-methylethoxy)methyl, butoxymethyl, (1-methylpropoxy)methyl, (2-methyl-propoxy)methyl, (1,1-dimethylethoxy)methyl, 2-(methoxy)ethyl, 2-(ethoxy)ethyl, 2-(propoxy)ethyl, 2-(1-methylethoxy)ethyl, 2-(butoxy)ethyl, 2-(1-methylpropoxy)-ethyl, 2-(2-methylpropoxy)ethyl, 2-(1,1-dimethylethoxy)ethyl, 2-(methoxy)propyl, 2-(ethoxy)propyl, 2-(propoxy)propyl, 2-(1-methylethoxy)propyl, 2-(butoxy)propyl, 2-(1-methylpropoxy)propyl, 2-(2-methylpropoxy)propyl, 2-(1,1-dimethylethoxy)-propyl, 3-(methoxy)propyl, 3-(ethoxy)propyl, 3-(propoxy)propyl, 3-(1-methylethoxy)-propyl, 3-(butoxy)propyl, 3-(1-methylpropoxy)propyl, 3-(2-methylpropoxy)propyl, 3-(1,1-dimethylethoxy)propyl, 2-(methoxy)butyl, 2-(ethoxy)butyl, 2-(propoxy)butyl, 2-(1-methylethoxy)butyl, 2-(butoxy)butyl, 2-(1-methylpropoxy)butyl, 2-(2-methyl-propoxy)butyl, 2-(1,1-dimethylethoxy)butyl, 3-(methoxy)butyl, 3-(ethoxy)butyl, 3-(propoxy)butyl, 3-(1-methylethoxy)butyl, 3-(butoxy)butyl, 3-(1-methylpropoxy)-butyl, 3-(2-methylpropoxy)butyl, 3-(1,1-dimethylethoxy)butyl, 4-(methoxy)butyl, 4-(ethoxy)butyl, 4-(propoxy)butyl, 4-(1-methylethoxy)butyl, 4-(butoxy)butyl, 4-(1-methylpropoxy)butyl, 4-(2-methylpropoxy)butyl and 4-(1,1-dimethylethoxy)butyl;
C₁-C₄-alkoxycarbonyl and also the alkoxycarbonyl moieties of C₁-C₄-alkoxycarbonyl-C₁-C₄-alkoxy, C₁-C₄-alkoxy-C₁-C₄-alkoxycarbonyl and di-(C₁-C₄-alkyl)amino-C₁-C₄-alkoxycarbonyl: for example methoxycarbonyl, ethoxycarbonyl, propoxycarbonyl, 1-methylethoxycarbonyl, butoxycarbonyl, 1-methylpropoxycarbonyl, 2-methylpropoxycarbonyl or 1,1-dimethylethoxycarbonyl;
C₁-C₆-alkoxycarbonyl and also the alkoxycarbonyl moieties of C₁-C₆-alkoxycarbonyl-C₁-C₆-alkoxy and C₁-C₆-alkoxycarbonylamino-C₁-C₄-alkyl: C₁-C₄-alkoxycarbonyl as mentioned above, and also, for example, pentoxycarbonyl, 1-methylbutoxycarbonyl, 2-methylbutoxycarbonyl, 3-methylbutoxycarbonyl, 2,2-dimethylpropoxycarbonyl, 1-ethylpropoxycarbonyl, hexoxycarbonyl, 1,1-dimethylpropoxycarbonyl, 1,2-dimethylpropoxycarbonyl, 1-methylpentoxycarbonyl, 2-methylpentoxycarbonyl, 3-methylpentoxycarbonyl, 4-methylpentoxycarbonyl, 1,1-dimethylbutoxycarbonyl, 1,2-dimethylbutoxycarbonyl, 1,3-dimethylbutoxycarbonyl, 2,2-dimethylbutoxycarbonyl, 2,3-dimethylbutoxycarbonyl, 3,3-dimethylbutoxycarbonyl, 1-ethylbutoxycarbonyl, 2-ethylbutoxycarbonyl, 1,1,2-trimethylpropoxycarbonyl, 1,2,2-trimethylpropoxycarbonyl, 1-ethyl-1-methylpropoxycarbonyl or 1-ethyl-2-methylpropoxycarbonyl;
C₁-C₄-alkylthio and also the C₁-C₄-alkylthio moieties of C₁-C₆-haloalkyl-C₁-C₄-thioalkyl, C₂-C₆-haloalkenyl-C₁-C₄-thioalkyl, C₂-C₆-haloalkynyl-C₁-C₄-thioalkyl: for example methylthio, ethylthio, propylthio, 1-methylethylthio, butylthio, 1-methylpropylthio, 2-methylpropylthio and 1,1-dimethylethylthio;
C₁-C₆-alkylthio and also the C₁-C₆-alkylthio moieties of C₁-C₆-alkylthio-C₁-C₄-alkyl: C₁-C₄-alkylthio as mentioned above, and also, for example, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutyl-thio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1 ethyl-2-methylpropylthio;
C₁-C₆-alkylamino and also the C₁-C₆-alkylamino radicals of N—(C₁-C₆-alkylamino)imino-C₁-C₆-alkyl, C₁-C₆-alkylamino-C₁-C₄-alkyl, C₁-C₆-alkylsulfonyl-(C₁-C₆-alkylamino)-C₁-C₄-alkyl, C₁-C₆-alkylcarbonyl-(C₁-C₆-alkylamino)-C₁-C₄-alkyl, [(C₁-C₆-alkyl)amino]cyanoimino and C₁-C₆-alkylaminocarbonyloxy-C₁-C₄-alkyl: for example methylamino, ethylamino, propylamino, 1-methylethylamino, butylamino, 1-methylpropylamino, 2-methylpropylamino, 1,1-dimethylethylamino, pentylamino, 1-methylbutylamino, 2-methyl butylamino, 3-methylbutylamino, 2,2-dimethylpropylamino, 1-ethylpropylamino, hexylamino, 1,1-dimethylpropylamino, 1,2-dimethylpropylamino, 1-methylpentylamino, 2-methylpentylamino, 3-methyl-pentylamino, 4-methylpentylamino, 1,1-dimethylbutylamino, 1,2-dimethylbutylamino, 1,3-dimethylbutylamino, 2,2-dimethylbutylamino, 2,3-dimethylbutylamino, 3,3-dimethylbutylamino, 1-ethylbutylamino, 2-ethylbutylamino, 1,1,2-trimethylpropyl-amino, 1,2,2-trimethylpropylamino, 1-ethyl-1-methylpropylamino or 1-ethyl-2-methyl-propylamino;
di(C₁-C₄-alkyl)amino: for example N,N-dimethylamino, N,N-diethylamino, N,N-dipropylamino, N,N-di-(1-methylethyl)amino, N,N-dibutylamino, N,N-di-(1-methylpropyl)amino, N,N-di-(2-methylpropyl)amino, N,N-di-(1,1-dimethyl-ethyl)amino, N-ethyl-N-methylamino, N-methyl-N-propylamino, N-methyl-N-(1-methylethyl)amino, N-butyl-N-methylamino, N-methyl-N-(1-methylpropyl)-amino, N-methyl-N-(2-methylpropyl)amino, N-(1,1-dimethylethyl)-N-methylamino, N-ethyl-N-propylamino, N-ethyl-N-(1-methylethyl)amino, N-butyl-N-ethylamino, N-ethyl-N-(1-methylpropyl)amino, N-ethyl-N-(2-methylpropyl)amino, N-ethyl-N-(1,1-dimethylethyl)amino, N-(1-methylethyl)-N-propylamino, N-butyl-N-propyl-amino, N-(1-methylpropyl)-N-propylamino, N-(2-methylpropyl)-N-propylamino, N-(1,1-dimethylethyl)-N-propylamino, N-butyl-N-(1-methylethyl)amino, N-(1-methyl-ethyl)-N-(1-methylpropyl)amino, N-(1-methylethyl)-N-(2-methylpropyl)amino, N-(1,1-dimethylethyl)-N-(1-methylethyl)amino, N-butyl-N-(1-methylpropyl)amino, N-butyl-N-(2-methylpropyl)amino, N-butyl-N-(1,1-dimethylethyl)amino, N-(1-methyl-propyl)-N-(2-methylpropyl)amino, N-(1,1-dimethylethyl)-N-(1-methylpropyl)amino and N-(1,1-dimethylethyl)-N-(2-methylpropyl)amino;
di(C₁-C₆-alkyl)amino and also the dialkylamino radicals of N-(di-C₁-C₆-alkylamino)-imino-C₁-C₆-alkyl, di(C₁-C₆-alkyl)amino-C₁-C₄-alkyl, [di(C₁-C₆-alkylamino)carbonyloxy]-C₁-C₄-alkyl, and [di(C₁-C₆-alkyl)amino]cyanoimino: di(C₁-C₄-alkyl)amino as mentioned above, and also, for example, N,N-dipentylamino, N,N-dihexylamino, N-methyl-N-pentylamino, N-ethyl-N-pentylamino, N-methyl-N-hexylamino and N-ethyl-N-hexylamino;
(C₁-C₄-alkylamino)carbonyl and also the (C₁-C₄-alkylamino)carbonyl moieties of (C₁-C₄-alkylamino)carbonylamino: for example methylaminocarbonyl, ethylaminocarbonyl, propylaminocarbonyl, 1-methylethylaminocarbonyl, butylaminocarbonyl, 1-methylpropylaminocarbonyl, 2-methylpropylaminocarbonyl or 1,1-dimethylethylaminocarbonyl;
di(C₁-C₄-alkyl)aminocarbonyl and also the di(C₁-C₄-alkyl)aminocarbonyl moieties of di(C₁-C₄-alkyl)aminocarbonylamino: for example N,N-dimethylaminocarbonyl, N,N-diethylaminocarbonyl, N,N-di-(1-methylethyl)aminocarbonyl, N,N-dipropyl-aminocarbonyl, N,N-dibutylaminocarbonyl, N,N-di-(1-methylpropyl)aminocarbonyl, N,N-di-(2-methylpropyl)aminocarbonyl, N,N-di-(1,1-dimethylethyl)aminocarbonyl, N-ethyl-N-methylaminocarbonyl, N-methyl-N-propylaminocarbonyl, N-methyl-N-(1-methylethyl)aminocarbonyl, N-butyl-N-methylaminocarbonyl, N-methyl-N-(1-methylpropyl)aminocarbonyl, N-methyl-N-(2-methylpropyl)aminocarbonyl, N-(1,1-dimethylethyl)-N-methylaminocarbonyl, N-ethyl-N-propylaminocarbonyl, N-ethyl-N-(1-methylethyl)aminocarbonyl, N-butyl-N-ethylaminocarbonyl, N-ethyl-N-(1-methylpropyl)aminocarbonyl, N-ethyl-N-(2-methylpropyl)aminocarbonyl, N-ethyl-N-(1,1-dimethylethyl)aminocarbonyl, N-(1-methylethyl)-N-propylamino-carbonyl, N-butyl-N-propylaminocarbonyl, N-(1-methylpropyl)-N-propylamino-carbonyl, N-(2-methylpropyl)-N-propylaminocarbonyl, N-(1,1-dimethylethyl)-N-propylaminocarbonyl, N-butyl-N-(1-methylethyl)aminocarbonyl, N-(1-methylethyl)-N-(1-methylpropyl)aminocarbonyl, N-(1-methylethyl)-N-(2-methylpropyl)amino-carbonyl, N-(1,1-dimethylethyl)-N-(1-methylethyl)aminocarbonyl, N-butyl-N-(1-methylpropyl)aminocarbonyl, N-butyl-N-(2-methylpropyl)aminocarbonyl, N-butyl-N-(1,1-dimethylethyl)aminocarbonyl, N-(1-methylpropyl)-N-(2-methyl-propyl)aminocarbonyl, N-(1,1-dimethylethyl)-N-(1-methylpropyl)aminocarbonyl or N-(1,1-dimethylethyl)-N-(2-methylpropyl)aminocarbonyl;
(C₁-C₆-alkylamino)carbonyl and also the (C₁-C₆-alkylamino)carbonyl moieties of (C₁-C₆-alkylamino)carbonylamino, C₁-C₆-alkylaminocarbonyl-C₁-C₄-alkyl and [(C₁-C₆-alkyl)aminocarbonylamino]C₁-C₄-alkyl: (C₁-C₄-alkylamino)carbonyl as mentioned above, and also, for example, pentylaminocarbonyl, 1-methylbutylaminocarbonyl, 2-methylbutylaminocarbonyl, 3-methylbutylaminocarbonyl, 2,2-dimethylpropylaminocarbonyl, 1-ethylpropylaminocarbonyl, hexylaminocarbonyl, 1,1-dimethylpropylaminocarbonyl, 1,2-dimethylpropylaminocarbonyl, 1-methylpentylaminocarbonyl, 2-methylpentyl-aminocarbonyl, 3-methylpentylaminocarbonyl, 4-methylpentylaminocarbonyl, 1,1-dimethylbutylaminocarbonyl, 1,2-dimethylbutylaminocarbonyl, 1,3-dimethylbutyl-aminocarbonyl, 2,2-dimethylbutylaminocarbonyl, 2,3-dimethylbutylaminocarbonyl, 3,3-dimethylbutylaminocarbonyl, 1-ethylbutylaminocarbonyl, 2-ethylbutylamino-carbonyl, 1,1,2-trimethylpropylaminocarbonyl, 1,2,2-trimethylpropylaminocarbonyl, 1-ethyl-1-methylpropylaminocarbonyl or 1-ethyl-2-methylpropylaminocarbonyl;
di(C₁-C₆-alkyl)aminocarbonyl and also the di(C₁-C₆-alkyl)aminocarbonyl moieties of di(C₁-C₆-alkyl)aminocarbonylamino, di(C₁-C₆-alkyl)aminocarbonyl-C₁-C₄-alkyl and [di(C₁-C₆-alkyl)aminocarbonylamino]C₁-C₄-alkyl: di(C₁-C₄-alkyl)aminocarbonyl as mentioned above, and also, for example, N-methyl-N-pentylaminocarbonyl, N-methyl-N-(1-methylbutyl)aminocarbonyl, N-methyl-N-(2-methylbutyl)aminocarbonyl, N-methyl-N-(3-methylbutyl)aminocarbonyl, N-methyl-N-(2,2-dimethylpropyl)amino-carbonyl, N-methyl-N-(1-ethylpropyl)aminocarbonyl, N-methyl-N-hexylamino-carbonyl, N-methyl-N-(1,1-dimethylpropyl)aminocarbonyl, N-methyl-N-(1,2-dimethyl-propyl)aminocarbonyl, N-methyl-N-(1-methylpentyl)aminocarbonyl, N-methyl-N-(2-methylpentyl)aminocarbonyl, N-methyl-N-(3-methylpentyl)aminocarbonyl, N-methyl-N-(4-methylpentyl)aminocarbonyl, N-methyl-N-(1,1-dimethylbutyl)amino-carbonyl, N-methyl-N-(1,2-dimethylbutyl)aminocarbonyl, N-methyl-N-(1,3-dimethyl-butyl)aminocarbonyl, N-methyl-N-(2,2-dimethylbutyl)aminocarbonyl, N-methyl-N-(2,3-dimethylbutyl)aminocarbonyl, N-methyl-N-(3,3-dimethylbutyl)aminocarbonyl N-methyl-N-(1-ethylbutyl)aminocarbonyl, N-methyl-N-(2-ethylbutyl)aminocarbonyl, N-methyl-N-(1,1,2-trimethylpropyl)aminocarbonyl, N-methyl-N-(1,2,2-trimethyl-propyl)aminocarbonyl, N-methyl-N-(1-ethyl-1-methylpropyl)aminocarbonyl, N-methyl-N-(1-ethyl-2-methylpropyl)aminocarbonyl, N-ethyl-N-pentylaminocarbonyl, N-ethyl-N-(1-methylbutyl)aminocarbonyl, N-ethyl-N-(2-methylbutyl)aminocarbonyl, N-ethyl-N-(3-methylbutyl)aminocarbonyl, N-ethyl-N-(2,2-dimethylpropyl)amino-carbonyl, N-ethyl-N-(1-ethylpropyl)aminocarbonyl, N-ethyl-N-hexylaminocarbonyl, N-ethyl-N-(1,1-dimethylpropyl)aminocarbonyl, N-ethyl-N-(1,2-dimethylpropyl)amino-carbonyl, N-ethyl-N-(1-methylpentyl)aminocarbonyl, N-ethyl-N-(2-methylpentyl)-aminocarbonyl, N-ethyl-N-(3-methylpentyl)aminocarbonyl, N-ethyl-N-(4-methyl-pentyl)aminocarbonyl, N-ethyl-N-(1,1-dimethylbutyl)aminocarbonyl, N-ethyl-N-(1,2-dimethylbutyl)aminocarbonyl, N-ethyl-N-(1,3-dimethylbutyl)aminocarbohyl, N-ethyl-N-(2,2-dimethylbutyl)aminocarbonyl, N-ethyl-N-(2,3-dimethylbutyl)amino-carbonyl, N-ethyl-N-(3,3-dimethylbutyl)aminocarbonyl, N-ethyl-N-(1-ethylbutyl)-aminocarbonyl, N-ethyl-N-(2-ethylbutyl)aminocarbonyl, N-ethyl-N-(1,1,2-trimethyl-propyl)aminocarbonyl, N-ethyl-N-(1,2,2-trimethylpropyl)aminocarbonyl, N-ethyl-N-(1-ethyl-1-methylpropyl)aminocarbonyl, N-ethyl-N-(1-ethyl-2-methylpropyl)-aminocarbonyl, N-propyl-N-pentylaminocarbonyl, N-butyl-N-pentylaminocarbonyl, N,N-dipentylaminocarbonyl, N-propyl-N-hexylaminocarbonyl, N-butyl-N-hexylamino-carbonyl, N-pentyl-N-hexylaminocarbonyl or N,N-dihexylaminocarbonyl;
di(C₁-C₆-alkyl)aminothiocarbonyl: for example N,N-dimethylaminothiocarbonyl, N,N-diethylaminothiocarbonyl, N,N-di-(1-methylethyl)aminothiocarbonyl, N,N-dipropylaminothiocarbonyl, N,N-dibutylaminothiocarbonyl, N,N-di-(1-methyl-propyl)aminothiocarbonyl, N,N-di-(2-methylpropyl)aminothiocarbonyl, N,N-di-(1,1-dimethylethyl)aminothiocarbonyl, N-ethyl-N-methylaminothiocarbonyl, N-methyl-N-propylaminothiocarbonyl, N-methyl-N-(1-methylethyl)aminothiocarbonyl, N-butyl-N-methylaminothiocarbonyl, N-methyl-N-(1-methylpropyl)aminothiocarbonyl, N-methyl-N-(2-methylpropyl)aminothiocarbonyl, N-(1,1-dimethylethyl)-N-methyl-aminothiocarbonyl, N-ethyl-N-propylaminothiocarbonyl, N-ethyl-N-(1-methylethyl)-aminothiocarbonyl, N-butyl-N-ethylaminothiocarbonyl, N-ethyl-N-(1-methylpropyl)-aminothiocarbonyl, N-ethyl-N-(2-methylpropyl)aminothiocarbonyl, N-ethyl-N-(1,1-dimethylethyl)aminothiocarbonyl, N-(1-methylethyl)-N-propylaminothio-carbonyl, N-butyl-N-propylaminothiocarbonyl, N-(1-methylpropyl)-N-propylamino-thiocarbonyl, N-(2-methylpropyl)-N-propylamino-thiocarbonyl, N-(1,1-dimethylethyl)-N-propylaminothiocarbonyl, N-butyl-N-(1-methylethyl)aminothiocarbonyl, N-(1-methylethyl)-N-(1-methylpropyl)aminothiocarbonyl, N-(1-methylethyl)-N-(2-methylpropyl)aminothiocarbonyl, N-(1,1-dimethylethyl)-N-(1-methylethyl)aminb-thiocarbonyl, N-butyl-N-(1-methylpropyl)aminothiocarbonyl, N-butyl-N-(2-methyl-propyl)aminothiocarbonyl, N-butyl-N-(1,1-dimethylethyl)aminothiocarbonyl, N-(1-methylpropyl)-N-(2-methylpropyl)aminothiocarbonyl, N-(1,1-dimethylethyl)-N-(1-methylpropyl)aminothiocarbonyl, N-(1,1-dimethylethyl)-N-(2-methylpropyl)-aminothiocarbonyl, N-methyl-N-pentylaminothiocarbonyl, N-methyl-N-(1-methyl-butyl)aminothiocarbonyl, N-methyl-N-(2-methylbutyl)aminothiocarbonyl, N-methyl-N-(3-methylbutyl)aminothiocarbonyl, N-methyl-N-(2,2-dimethylpropyl)amino-thiocarbonyl, N-methyl-N-(1-ethylpropyl)aminothiocarbonyl, N-methyl-N-hexylamino-thiocarbonyl, N-methyl-N-(1-dimethylpropyl)aminothiocarbonyl, N-methyl-N-(1,2-dimethylpropyl)aminothiocarbonyl, N-methyl-N-(1-methylpentyl)-aminothiocarbonyl, N-methyl-N-(2-methylpentyl)aminothiocarbonyl, N-methyl-N-(3-methylpentyl)aminothiocarbonyl, N-methyl-N-(4-methylpentyl)aminothio-carbonyl, N-methyl-N-(1,1-dimethylbutyl)aminothiocarbonyl, N-methyl-N-(1,2-dimethylbutyl)aminothiocarbonyl, N-methyl-N-(1,3-dimethylbutyl)amino-thiocarbonyl, N-methyl-N-(2,2-dimethylbutyl)aminothiocarbonyl, N-methyl-N-(2,3-dimethylbutyl)aminothiocarbonyl, N-methyl-N-(3,3-dimethylbutyl)aminothio-carbonyl, N-methyl-N-(1-ethylbutyl)aminothiocarbonyl, N-methyl-N-(2-ethylbutyl)-aminothiocarbonyl, N-methyl-N-ethyl-N-(1,1,2-trimethylpropyl)aminothiocarbonyl, N-methyl-N-(1,2,2-trimethylpropyl)aminothiocarbonyl, N-methyl-N-(1-ethyl-1-methyl-propyl)aminothiocarbonyl, N-methyl-N-(1-ethyl-2-methylpropyl)aminothiocarbonyl, N-ethyl-N-pentylaminothiocarbonyl, N-ethyl-N-(1-methylbutyl)aminothiocarbonyl, N-ethyl-N-(2-methylbutyl)aminothiocarbonyl, N-ethyl-N-(3-methylbutyl)aminothio-carbonyl, N-ethyl-N-(2,2-dimethylpropyl)aminothiocarbonyl, N-ethyl-N-(1-ethyl-propyl)aminothiocarbonyl, N-ethyl-N-hexylaminothiocarbonyl, N-ethyl-N-(1,1-dimethylpropyl)aminothiocarbonyl, N-ethyl-N-(1,2-dimethylpropyl)amino-thiocarbonyl, N-ethyl-N-(1-methylpentyl)aminothiocarbonyl, N-ethyl-N-(2-methyl-pentyl)aminothiocarbonyl, N-ethyl-N-(3-methylpentyl)aminothiocarbonyl, N-ethyl-N-(4-methylpentyl)aminothiocarbonyl, N-ethyl-N-(1,1-dimethylbutyl)aminothio-carbonyl, N-ethyl-N-(1,2-dimethylbutyl)aminothiocarbonyl, N-ethyl-N-(1,3-dimethyl-butyl)aminothiocarbonyl, N-ethyl-N-(2,2-dimethylbutyl)aminothiocarbonyl, N-ethyl-N-(2,3-dimethylbutyl)aminothiocarbonyl, N-ethyl-N-(3,3-dimethylbutyl)amino-thiocarbonyl, N-ethyl-N-(1-ethylbutyl)aminothiocarbonyl, N-ethyl-N-(2-ethylbutyl)-aminothiocarbonyl, N-ethyl-N-(1,1,2-trimethylpropyl)aminothiocarbonyl, N-ethyl-N-(1,2,2-trimethylpropyl)aminothiocarbonyl, N-ethyl-N-(1-ethyl-1-methylpropyl)-aminothiocarbonyl, N-ethyl-N-(1-ethyl-2-methylpropyl)aminothiocarbonyl, N-propyl-N-pentylaminothiocarbonyl, N-butyl-N-pentylaminothiocarbonyl, N,N-dipentyl-aminothiocarbonyl, N-propyl-N-hexylaminothiocarbonyl, N-butyl-N-hexylaminothio-carbonyl, N-pentyl-N-hexylaminothiocarbonyl or N,N-dihexylaminothiocarbonyl;
three- to six-membered heterocyclyl: monocyclic saturated or partially unsaturated hydrocarbons having three to six ring members as mentioned above which, in addition to carbon atoms, may contain one to four nitrogen atoms or one to three nitrogen atoms and one oxygen or sulfur atom or one to three oxygen atoms or one to three sulfur atoms and which may be attached via a carbon atom or a nitrogen atom, for example for example 2-oxiranyl, 2-oxetanyl, 3-oxetanyl, 2-aziridinyl, 3-thiethanyl, 1-azetidinyl, 2-azetidinyl,
for example 2-tetrahydrofuranyl, 3-tetrahydrofuranyl, 2-tetrahydrothienyl, 3-tetra-hydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 1,2,3,4-tetrazolidin-5-yl;
for example 1-pyrrolidinyl, 2-isothiazolidinyl, 2-isothiazolidinyl, 1-pyrazolidinyl, 3-oxazolidinyl, 3-thiazolidinyl, 1-imidazolidinyl, 1,2,4-triazolidin-1-yl, 1,2,4-oxadiazolidin-2-yl, 1,2,4-oxadiazolidin-4-yl, 1,2,4-thiadiazolidin-2-yl, 1,2,4-thiadiazolidin-4-yl, 1,2,3,4-tetrazolidin-1-yl,
for example 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 4,5-dihydropyrrol-2-yl, 4,5-dihydropyrrol-3-yl, 2,5-dihydropyrrol-2-yl, 2,5-dihydropyrrol-3-yl, 4,5-dihydroisoxazol-3-yl, 2,5-dihydroisoxazol-3-yl, 2,3-dihydroisoxazol-3-yl, 4,5-dihydroisoxazol-4-yl, 2,5-dihydroisoxazol-4-yl, 2,3-dihydroisoxazol-4-yl, 4,5-dihydroisoxazol-5-yl, 2,5-dihydroisoxazol-5-yl, 2,3-dihydroisoxazol-5-yl, 4,5-dihydroisothiazol-3-yl, 2,5-dihydroisothiazol-3-yl, 2,3-dihydroisothiazol-3-yl, 4,5-dihydroisothiazol-4-yl, 2,5-dihydroisothiazol-4-yl, 2,3-dihydroisothiazol-4-yl, 4,5-dihydroisothiazol-5-yl, 2,5-dihydroisothiazol-5-yl, 2,3-dihydroisothiazol-5-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-3-yl, 3,4-dihydropyrazol-4-yl, 3,4-dihydropyrazol-5-yl, 4,5-dihydropyrazol-3-yl, 4,5-dihydropyrazol-4-yl, 4,5-dihydropyrazol-5-yl, 2,3-dihydroimidazol-2-yl, 2,3-dihydroimidazol-3-yl, 2,3-dihydroimidazol-4-yl, 2,3-dihydroimidazol-5-yl, 4,5-dihydroimidazol-2-yl, 4,5-dihydroimidazol-4-yl, 4,5-dihydroimidazol-5-yl, 2,5-dihydroimidazol-2-yl, 2,5-dihydroimidazol-4-yl, 2,5-dihydroimidazol-5-yl, 2,3-dihydrooxazol-3-yl, 2,3-dihydrooxazol-4-yl, 2,3-dihydrooxazol-5-yl, 3,4-dihydrooxazol-3-yl, 3,4-dihydrooxazol-4-yl, 3,4-dihydrooxazol-5-yl, 2,3-dihydrothiazol-3-yl, 2,3-dihydrothiazol-4-yl, 2,3-di-hydrothiazol-5-yl, 3,4-dihydrothiazol-3-yl, 3,4-dihydrothiazol-4-yl, 3,4-dihydrothiazol-5-yl, 3,4-dihydrothiazol-2-yl, 3,4-dihydrothiazol-3-yl, 3,4-dihydrothiazol-4-yl,
for example 4,5-dihydropyrrol-1-yl, 2,5-dihydropyrrol-1-yl, 4,5-dihydroisoxazol-2-yl, 2,3-dihydroisoxazol-1-yl, 4,5-dihydroisothiazol-1-yl, 2,3-dihydroisothiazol-1-yl, 2,3-dihydropyrazol-1-yl, 4,5-dihydropyrazol-1-yl, 3,4-dihydropyrazol-1-yl, 2,3-dihydro-imidazol-1-yl, 4,5-dihydroimidazol-1-yl, 2,5-dihydroimidazol-1-yl, 2,3-dihydrooxazol-2-yl, 3,4-dihydrooxazol-2-yl, 2,3-dihydrothiazol-2-yl, 3,4-dihydrothiazol-2-yl;
for example 2-piperidinyl, 3-piperidinyl, 4-piperidinyl, 1,3-dioxan-2-yl, 1,3-dioxan-4-yl, 1,3-dioxan-5-yl, 1,4-dioxan-2-yl, 1,3-dithian-2-yl, 1,4-dithiane-3-yl, 1,3-dithian-4-yl, 1-4,dithian-2-yi 2-tetrahydropyranyl, 3-tetrahydropyranyl, 4-tetrahydrothiopyranyl, 2-tetrahydro-thiopyranyl, 3-tetrahydrothiopyranyl, 4-tetrahydrothiopyranyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl, 1,2,4-hexahydrotriazin-3-yl, tetrahydro-1,3-oxazin-2-yl, tetrahydro-1,3-oxazin-6-yl, 2-morpholinyl, 3-morpholinyl, 1,3,5-trioxan-2-yl;
for example 1-piperidinyl, 1-hexahydropyridazinyl, 1-hexahydropyrimidinyl, 1-piperazinyl, 1,3,5-hexahydrotriazin-1-yl, 1,2,4-hexahydrotriazin-1-yl, tetrahydro-1,3-oxazin-1-yl, 1-morpholinyl;
for example 2H-pyran-2-yl, 2H-pyran-3-yl, 2H-pyran-4-yl, 2H-pyran-5-yl, 2H-pyran-6-yl, 3,6-dihydro-2H-pyran-2-yl, 3,6-dihydro-2H-pyran-3-yl, 3,6-dihydro-2H-pyran-4-yl, 3,6-dihydro-2H-pyran-5-yl, 3,6-dihydro-2H-pyran-6-yl, 3,4-dihydro-2H-pyran-3-yl, 3,4-dihydro-2H-pyran-4-yl, 3,4-dihydro-2H-pyran-6-yl, 2H-thiopyran-2-yl, 2H-thiopyran-3-yl, 2H-thiopyran-4-yl, 2H-thiopyran-5-yl, 2H-thiopyran-6-yl, 5,6-dihydro-4H-1,3-oxazin-2-yl;
aryl and the aryl moiety of aryl-(C₁-C₄-alkyl): a monocyclic to tricyclic aromatic carbocycle having 6 to 14 ring members, such as, for example, phenyl, naphthyl and anthracenyl;
heteroaryl and also the heteroaryl radicals in heteroaryl-C₁-C₄-alkyl, heteroaryl-C₁-C₄-alkyl, heteroaryl-C₂-C₄-alkenyl, heteroaryl-C₂-C₄-alkynyl, heteroaryl-C₁-C₄-haloalkyl, heteroaryl-C₂-C₄-haloalkenyl, heteroaryl-C₂-C₄-haloalkynyl, heteroaryl-C₁-C₄-hydroxyalkyl, heteroaryl-C₂-C₄-hydroxyalkenyl, heteroaryl-C₂-C₄-hydroxyalkynyl, heteroarylcarbonyl-C₁-C₄-alkyl, heteroarylcarbonyloxy-C₁-C₄-alkyl, heteroaryloxycarbonyl-C₁-C₄-alkyl, heteroaryloxy-C₁-C₄-alkyl, heteroarylthio-C₁-C₄-alkyl, heteroarylsulfinyl-C₁-C₄-alkyl, heteroarylsulfonyl-C₁-C₄-alkyl:
mono- or bicyclic aromatic heteroaryl having 5 to 10 ring members which, in addition to carbon atoms, comprises 1 to 4 nitrogen atoms, or 1 to 3 nitrogen atoms and an oxygen or sulfur atom, or an oxygen or a sulfur atom, for example
monocycles, such as furyl (for example 2-furyl, 3-furyl), thienyl (for example 2-thienyl, 3-thienyl), pyrrolyl (for example pyrrol-2-yl, pyrrol-3-yl), pyrazolyl (for example pyrazol-3-yl, pyrazol-4-yl), isoxazolyl (for example isoxazol-3-yl, isoxazol-4-yl, isoxazol-5-yl), isothiazolyl (for example isothiazol-3-yl, isothiazol-4-yl, isothiazol-5-yl), imidazolyl (for example imidazol-2-yl, imidazol-4-yl), oxazolyl (for example oxazol-2-yl, oxazol yl, oxazol-5-yl), thiazolyl (for example thiazol-2-yl, thiazol-4-yl, thiazol-5-yl), oxadiazolyl (for example 1,2,3-oxadiazol-4-yl, 1,2,3-oxadiazol-5-yl, 1,2,4-oxadiazol-3-yl, 1,2,4,-oxadiazol-5-yl, 1,3,4-oxadiazol-2-yl), thiadiazolyl (for example 1,2,3-thiadiazol yl, 1,2,3-thiadiazol-5-yl, 1,2,4-thiadiazol-3-yl, 1,2,4-thiadiazol-5-yl, 1,3,4-thiadiazolyl-2-yl), triazolyl (for example 1,2,3-triazol-4-yl, 1,2,4-triazol-3-yl), tetrazol-5-yl, pyridyl (for example pyridin-2-yl, pyridin-3-yl, pyridin-4-yl), pyrazinyl (for example pyridazin-3-yl, pyridazin-4-yl), pyrimidinyl (for example pyrimidin-2-yl, pyrimidin-4-yl, pyrimidin-5-yl), pyrazin-2-yl, triazinyl (for example 1,3,5-triazin-2-yl, 1,2,4-triazin-3-yl, 1,2,4-triazin-5-yl, 1,2,4-triazin-6-yl), tetrazinyl (for example 1,2,4,5-tetrazin-3-yl); and also bicycles such as the benzo-fused derivatives of the abovementioned monocycles, for example quinolinyl, isoquinolinyl, indolyl, benzothienyl, benzofuranyl, benzoxazolyl, benzothiazolyl, benzisothiazolyl, benzimidazolyl, benzopyrazolyl, benzothiadiazolyl, benzotriazolyl.

In a particular embodiment, the variables of the benzoyl-substituted alanines of the formula I have the following meanings which, both on their own and in combination with one another are particular embodiments of the compounds of the formula I:

Preference is given to the benzoyl-substituted alanines of the formula I in which
R¹ is halogen, C₁-C₄-alkyl or C₁-C₆-haloalkyl;
   particularly preferably halogen or C₁-C₆-haloalkyl;
   especially preferably halogen or C₁-C₄-haloalkyl;
   extremely preferably fluorine, chlorine or CF₃.

Preference is also given to the benzoyl-substituted alanines of the formula I in which
R² and R³, independently of one another, are
   hydrogen, halogen, C₁-C₄-alkyl or C₁-C₆-haloalkyl;
   very preferably hydrogen, halogen or C₁-C₆-haloalkyl;
   particularly preferably hydrogen, halogen or C₁-C₄-haloalkyl;
   especially preferably hydrogen, fluorine, chlorine or CF₃;
   extremely preferably hydrogen, fluorine or chlorine;
   most extremely preferably hydrogen or fluorine.

Preference is also given to the benzoyl-substituted alanines of the formula I in which
R⁴ is hydrogen, halogen, C₁-C₄-alkyl or C₁-C₄-haloalkyl;
   particularly preferably hydrogen, halogen or C₁-C₄-alkyl;
   especially preferably hydrogen or halogen;
   extremely preferably hydrogen.

Preference is also given to the benzoyl-substituted alanines of the formula I in which
R⁵ is hydrogen, halogen, C₁-C₄-alkyl or C₁-C₄-haloalkyl;
   particularly preferably hydrogen, halogen or C₁-C₄-alkyl;
   especially preferably hydrogen or halogen;
   extremely preferably hydrogen.

Preference is also given to the benzoyl-substituted alanines of the formula I in which
R⁶ is hydrogen.

Preference is also given to the benzoyl-substituted alanines of the formula I in which R⁷ is hydrogen or hydroxyl;
particularly preferably hydrogen.

Preference is also given to the benzoyl-substituted alanines of the formula I in which
R⁶ is hydrogen; and
R⁷ is hydrogen or hydroxyl;
   particularly preferably hydrogen.

Preference is also given to the benzoyl-substituted alanines of the formula I in which
R⁸ is C₁-C₆-alkyl or C₁-C₆-haloalkyl;
   particularly preferably C₁-C₆-alkyl;
   especially preferably C₁-C₄-alkyl;
   most preferably CH₃.

Preference is also given to the benzoyl-substituted alanines of the formula I in which
R⁹ is hydrogen or C₁-C₄-alkyl;
   preferably hydrogen or CH₃;
   especially preferably hydrogen.

Preference is also given to the benzoyl-substituted alanines of the formula I, in which
R¹⁰ is hydrogen, C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₁-C₆-cyanoalkyl, C₂-C₆-cyanoalkenyl, C₂-C₆-cyanoalkynyl, C₁-C₆-hydroxyalkyl, C₂-C₆-hydroxyalkenyl, C₂-C₆-hydroxyalkynyl, C₃-C₆-cycloalkyl, C₃-C₆-cycloalkenyl, 3- to 6-membered heterocyclyl,
   where the cycloalkyl, cycloalkenyl or 3- to 6-membered heterocyclyl radicals mentioned above may be partially or fully halogenated and/or may carry one to three radicals from the group consisting of oxo, cyano, nitro, C₁-C₆-alkyl, C₁-C₆-haloalkyl, hydroxyl, C₁-C₆-alkoxy, C₁-C₆-haloalkoxy, hydroxycarbonyl, C₁-C₆-alkoxycarbonyl, hydroxycarbonyl-C₁-C₆-alkoxy, C₁-C₆-alkoxycarbonyl-C₁-C₆-alkoxy, amino, C₁-C₆-alkylamino, di(C₁-C₆-alkyl)amino, C₁-C₆-alkylsulfonylamino, C₁-C₆-haloalkylsulfonylamino, aminocarbonylamino, (C₁-C₆-alkylamino)carbonylamino, di(C₁-C₆-alkyl)aminocarbonylamino, aryl and aryl(C₁-C₆-alkyl);
   C₁-C₆-alkoxy-C₁-C₄-alkyl, C₂-C₆-alkenyloxy-C₁-C₄-alkyl, C₂-C₆-alkynyloxy-C₁-C₄-alkyl, C₁-C₆-haloalkoxy-C₁-C₄-alkyl, C₂-C₆-haloalkenyloxy-C₁-C₄-alkyl, C₂-C₆-haloalkynyloxy-C₁-C₄-alkyl, C₁-C₆-alkoxy-C₁-C₄-alkoxy-C₁-C₄-alkyl, C₁-C₆-alkylthio-C₁-C₄-alkyl, C₂-C₆-alkenylthio-C₁-C₄-alkyl, C₂-C₆-alkynylthio-C₁-C₄-alkyl, C₁-C₆-haloalkyl-C₁-C₄-thioalkyl, C₂-C₆-haloalkenyl-C₁-C₄-thioalkyl, C₂-C₆-haloalkynyl-C₁-C₄-thioalkyl, C₁-C₆-alkylsulfinyl-C₁-C₄-alkyl, C₁-C₆-haloalkylsulfinyl-C₁-C₄-alkyl, C₁-C₆-alkylsulfonyl-C₁-C₄-alkyl, C₁-C₆-haloalkylsulfonyl-C₁-C₄-alkyl, amino-C₁-C₄-alkyl, C₁-C₆-alkylamino-C₁-C₄-alkyl, di(C₁-C₆-alkyl)amino-C₁-C₄-alkyl, C₁-C₆-alkylsulfonylamino-C₁-C₄-alkyl, C₁-C₆-alkylsulfonyl(C₁-C₆-alkyl)amino-C₁-C₄-alkyl, C₁-C₆-alkylcarbonyl, hydroxycarbonyl, C₁-C₆-alkoxycarbonyl, aminocarbonyl, C₁-C₆-alkylaminocarbonyl, di(C₁-C₆-alkyl)aminocarbonyl, formylamino-C₁-C₄-alkyl, C₁-C₆-alkoxycarbonylamino-C₁-C₄-alkyl, C₁-C₆-alkylcarbonyl-C₁-C₆-alkyl, hydroxycarbonyl-C₁-C₄-alkyl, C₁-C₆-alkoxycarbonyl-C₁-C₄-alkyl, C₁-C₆-haloalkoxycarbonyl-C₁-C₄-alkyl, C₁-C₆-alkylcarbonyloxy-C₁-C₄-alkyl, aminocarbonyl-C₁-C₄-alkyl, C₁-C₆-alkylaminocarbonyl-C₁-C₄-alkyl, di(C₁-C₆-alkyl)aminocarbonyl-C₁-C₄-alkyl, C₁-C₆-alkylcarbonylamino-C₁-C₄-alkyl, C₁-C₆-alkylcarbonyl-(C₁-C₆-alkylamino)-C₁-C₄-alkyl, di(C₁-C₆-alkyl)aminocarbonyloxy-C₁-C₄-alkyl, [(C₁-C₆-alkyl)amino-carbonylamino]C₁-C₄-alkyl, [di(C₁-C₆-alkyl)aminocarbonylamino]C₁-C₄-alkyl;
   phenyl-C₁-C₄-alkyl, phenyl-C₂-C₄-alkenyl, phenyl-C₂-C₄-alkynyl, phenyl-C₁-C₄-haloalkyl, phenyl-C₂-C₄-haloalkenyl, phenyl-C₂-C₄-haloalkynyl, phenyl-C₁-C₄-hydroxyalkyl, phenyl-C₂-C₄-hydroxyalkenyl, phenyl-C₂-C₄-hydroxyalkynyl, phenylcarbonyl-C₁-C₄-alkyl, phenylcarbonyloxy-C₁-C₄-alkyl, phenyloxycarbonyl-C₁-C₄-alkyl, phenyloxy-C₁-C₄-alkyl, phenylthio-C₁-C₄-alkyl, phenylsulfinyl-C₁-C₄-alkyl, phenylsulfonyl-C₁-C₄-alkyl,
   heteroaryl, heteroaryl-C₁-C₄-alkyl, heteroaryl-C₂-C₄-alkenyl, heteroaryl-C₂-C₄-alkyn-yl, heterQaryl-C₁-C₄-haloalkyl, heteroaryl-C₂-C₄-haioalkenyl, heteroaryl-C₂-C₄-haloalkynyl, heteroaryl-C₁-C₄-hydroxyalkyl, heteroaryl-C₂-C₄-hydroxyalkenyl,
   heteroaryl-C₂-C₄-hydroxyalkynyl, heteroarylcarbonyl-C₁-C₄-alkyl, heteroarylcarbonyloxy-C₁-C₄-alkyl, heteroaryloxycarbonyl-C₁-C₄-alkyl, heteroaryloxy-C₁-C₄-alkyl, heteroarylthio-C₁-C₄-alkyl, heteroarylsulfinyl-C₁-C₄-alkyl, heteroarylsulfonyl-C₁-C₄-alkyl,
      where the phenyl and heteroaryl radicals mentioned above may be partially or fully halogenated and/or may carry one to three radicals from the group consisting of cyano, nitro, C₁-C₆-alkyl, C₁-C₆-haloalkyl, hydroxyl, C₁-C₆-hydroxyalkyl, C₁-C₆-alkoxy, C₁-C₆-haloalkoxy, hydroxycarbonyl, C₁-C₆-alkoxycarbonyl, hydroxycarbonyl-C₁-C₆-alkoxy, C₁-C₆-alkoxycarbonyl-C₁-C₆-alkoxy, amino, C₁-C₆-alkylamino, di(C₁-C₆-alkyl)amino, C₁-C₆-alkylsulfonylamino, C₁-C₆-haloalkylsulfonylamino, (C₁-C₆-alkylamino)-carbonylamino, di(C₁-C₆-alkyl)aminocarbonylamino, aryl and aryl(C₁-C₆-alkyl).

Preference is also given to the benzoyl-substituted alanines of the formula I in which
R¹⁰ is hydrogen, C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₁-C₆-cyanoalkyl, C₁-C₆-hydroxyalkyl, C₂-C₆-hydroxyalkenyl, C₂-C₆-hydroxyalkynyl, C₃-C₆-cycloalkyl, C₃-C₆-cycloalkenyl, 3- to 6-membered heterocyclyl,
   where the cycloalkyl, cycloalkenyl or 3- to 6-membered heterocyclyl radicals mentioned above may be partially or fully halogenated and/or may carry one to three radicals from the group consisting of oxo, C₁-C₆-alkyl, C₁-C₆-haloalkyl, hydroxycarbonyl and C₁-C₆-alkoxycarbonyl;
   C₁-C₆-alkoxy-C₁-C₄-alkyl, C₁-C₆-haloalkoxy-C₁-C₄-alkyl, C₁-C₆-alkoxy-C₁-C₄-alkoxy-C₁-C₄-alkyl, C₁-C₆-alkylthio-C₁-C₄-alkyl, C₁-C₆-alkylsulfonylamino-C₁-C₄-alkyl, hydroxycarbonyl, C₁-C₆-alkoxycarbonyl, hydroxycarbonyl-C₁-C₄-alkyl, C₁-C₆-alkoxycarbonyl-C₁-C₄-alkyl, C₁-C₆-haloalkoxycarbonyl-C₁-C₄-alkyl, C₁-C₆-alkylcarbonyloxy-C₁-C₄-alkyl, C₁-C₆-alkylcarbonylamino-C₁-C₄-alkyl, [(C₁-C₆-alkyl)aminocarbonylamino]C₁-C₄-alkyl, di(C₁-C₆-alkyl)aminocarbonylamino-C₁-C₄-alkyl, [di(C₁-C₆-alkylamino)carbonyloxy]C₁-C₄-alkyl, formylamino-C₁-C₄-alkyl; phenyl-C₁-C₄-alkyl, phenyl-C₂-C₄-alkenyl, phenyl-C₂-C₄-alkynyl, phenyl-C₁-C₄-haloalkyl, phenyl-C₂-C₄-haloalkenyl, phenyl-C₁-C₄-hydroxyalkyl, phenyloxy-C₁-C₄-alkyl, phenylthio-C₁-C₄-alkyl, phenylsulfinyl-C₁-C₄-alkyl, phenylsulfonyl-C₁-C₄-alkyl;
   heteroaryl, heteroaryl-C₁-C₄-alkyl, heteroaryl-C₁-C₄-hydroxyalkyl, heteroaryloxy-C₁-C₄-alkyl, heteroarylthio-C₁-C₄-alkyl, heteroarylsulfinyl-C₁-C₄-alkyl or heteroarylsulfonyl-C₁-C₄-alkyl,
      where the phenyl and heteroaryl radicals mentioned above may be partially or fully halogenated and/or may carry one to three radicals from the group consisting of cyano, nitro, C₁-C₆-alkyl, C₁-C₆-haloalkyl, hydroxyl, C₁-C₆-alkoxy, C₁-C₆-haloalkoxy, hydroxycarbonyl, C₁-C₆-alkoxycarbonyl, hydroxycarbonyl-C₁-C₆-alkoxy, C₁-C₆-alkylsulfonylamino and C₁-C₆-haloalkylsulfonylamino;
   particularly preferably hydrogen, C₁-C₆-alkyl, C₂-C₆-alkenyl, C₂-C₆-alkynyl, C₁-C₆-haloalkyl, C₂-C₆-haloalkenyl, C₁-C₆-hydroxyalkyl, C₁-C₆-alkoxy-C₁-C₄-alkyl, C₁-C₆-haloalkoxy-C₁-C₄-alkyl, hydroxycarbonyl-C₁-C₄-alkyl, C₁-C₆-alkoxycarbonyl-C₁-C₄-alkyl, C₁-C₆-alkylcarbonyloxy-C₁-C₄-alkyl, C₁-C₆-alkylcarbonylamino-C₁-C₄-alkyl, [di(C₁-C₆-alkyl)amino)carbonyloxy]C₁-C₄-alkyl, formylamino-C₁-C₄-alkyl, phenyl-C₁-C₄-alkyl, phenyl-C₂-C₄-alkenyl, phenyl-C₂-C₄-alkynyl, phenyl-C₁-C₄-haloalkyl, phenyl-C₂-C₄-haloalkenyl, phenyl-C₁-C₄-hydroxyalkyl, phenyloxy-C₁-C₄-alkyl, phenylthio-C₁-C₄-alkyl, phenylsulfinyl-C₁-C₄-alkyl or phenylsulfonyl-C₁-C₄-alkyl,
      where the phenyl radicals mentioned above may be partially or fully halogenated and/or may carry one to three radicals from the group consisting of C₁-C₆-alkyl, C₁-C₆-haloalkyl, C₁-C₆-alkoxy, hydroxycarbonyl, C₁-C₆-alkoxycarbonyl, C₁-C₆-alkylsulfonylamino and C₁-C₆-haloalkylsulfonylamino;
   especially preferably hydrogen, C₁-C₆-alkyl, C₂-C₆-alkenyl, C₂-C₆-alkynyl, C₁-C₆-haloalkyl, C₂-C₆-haloalkenyl, C₁-C₆-hydroxyalkyl, hydroxycarbonyl-C₁-C₄-alkyl, C₁-C₆-alkoxycarbonyl-C₁-C₄-alkyl, [di(C₁-C₆-alkyl)aminocarbonyloxy]-C₁-C₄-alkyl, formylamino-C₁-C₄-alkyl; phenyl-C₁-C₄-alkyl, phenyl-C₂-C₄-alkenyl, phenyl-C₁-C₄-hydroxyalkyl or phenylthio-C₁-C₄-alkyl;
   most preferably hydrogen, C₁-C₆-alkyl, C₂-C₆-alkenyl, C₁-C₆-haloalkyl, C₂-C₆-haloalkenyl, C₁-C₆-hydroxyalkyl, formylamino-C₁-C₄-alkyl, phenyl-C₁-C₄-alkyl or phenyl-C₁-C₄-hydroxyalkyl.

Preference is also given to the benzoyl-substituted alanines of the formula I in which
R¹¹ is hydrogen, C₁-C₆-alkyl, C₃-C₆-cycloalkyl, C₃-C₆-alkenyl, C₃-C₆-alkynyl, C₃-C₆-haloalkenyl, C₃-C₆-haloalkynyl, formyl, C₁-C₆-alkylcarbonyl, C₃-C₆-cycloalkyl-carbonyl, C₂-C₆-alkenylcarbonyl, C₂-C₆-alkynylcarbonyl, C₁-C₆-alkoxycarbonyl, C₃-C₆-alkenyloxycarbonyl, C₃-C₆-alkynyloxycarbonyl, C₁-C₆-lkylaminocarbonyl, C₃-C₆-alkenylaminocarbonyl, C₃-C₆-alkynylaminocarbonyl, C₁-C₆-alkylsulfonyl-aminocarbonyl, di(C₁-C₆-alkyl)aminocarbonyl, N—(C₃-C₆-alkenyl)-N—(C₁-C₆-alkyl)-aminocarbonyl, N—(C₃-C₆-alkynyl)-N—(C₁-C₆-alkyl)aminocarbonyl, N—(C₁-C₆-alkoxy)-N—(C₁-C₆-alkyl)aminocarbonyl, N—(C₃-C₆-alkenyl)-N—(C₁-C₆-alkoxy)amino-carbonyl, N—(C₃-C₆-alkynyl)-N—(C₁-C₆-alkoxy)aminocarbonyl, di(C₁-C₆-alkyl)-aminothiocarbonyl, (C₁-C₆-alkyl)cyanoimino, (amino)cyanoimino, [(C₁-C₆-alkyl)amino]cyanoimino, di(C₁-C₆-alkyl)aminocyanoimino, C₁-C₆-alkylcarbonyl-C₁-C₆-alkyl, C₁-C₆-alkoxyimino-C₁-C₆-alkyl, N—(C₁-C₆-alkylamino)-imino-C₁-C₆-alkyl, N-[di(C₁-C₆-alkyl)amino]imino-C₁-C₆-alkyl or tri-C₁-C₄-alkylsilyl,
   where the alkyl, cycloalkyl and alkoxy radicals mentioned may be partially or fully halogenated and/or may carry one to three of the following groups: cyano, hydroxyl, C₃-C₆-cycloalkyl, C₁-C₆-alkoxy-C₁-C₄-alkyl, C₁-C₄-alkoxy-C₁-C₄-alkoxy-C₁-C₄-alkyl, C₁-C₄-alkoxy, C₁-C₄-alkylthio, di(C₁-C₄-alkyl)-amino, C₁-C₄-alkyl-C₁-C₆-alkoxycarbonylamino, C₁-C₄-alkylcarbonyl, hydroxycarbonyl, C₁-C₄-alkoxycarbonyl, aminocarbonyl, C₁-C₄-alkylamino-carbonyl, di(C₃-C₄-alkyl)aminocarbonyl or C₁-C₄-alkylcarbonyloxy; phenyl, phenyl-C₁-C₆-alkyl, phenylcarbonyl-C₁-C₆-alkyl, phenoxycarbonyl, phenylaminocarbonyl, phenylsulfonylaminocarbonyl, N—(C₁-C₆-alkyl)-N-(phenyl)-aminocarbonyl, phenyl-C₁-C₆-alkylcarbonyl,
   where the phenyl radical may be partially or fully halogenated and/or may carry one to three of the following groups: nitro, cyano, C₁-C₄-alkyl, C₁-C₄-haloalkyl, C₁-C₄-alkoxy or C₁-C₄-haloalkoxy; or
   SO₂R¹³.

Preference is also given to the benzoyl-substituted alanines of the formula I in which
R¹¹ is hydrogen, C₁-C₆-alkyl, C₃-C₆-alkenyl, C₃-C₆-alkynyl, formyl, C₁-C₆-alkylcarbonyl, C₂-C₆-alkenylcarbonyl, C₃-C₆-cycloalkylcarbonyl, C₁-C₆-alkoxycarbonyl, C₁-C₆-alkylaminocarbonyl, C₁-C₆-alkylsulfonylaminocarbonyl, di-(C₁-C₆-alkyl)aminocarbonyl, N—(C₁-C₆-alkoxy)-N—(C₁-C₆-alkyl)aminocarbonyl, di-(C₁-C₆-alkyl)aminothicarbonyl, C₁-C₆-alkoxyimino-C₁-C₆-alkyl,
   where the alkyl, cycloalkyl and alkoxy radicals mentioned may be partially or fully halogenated and/or may carry one to three of the following groups: cyano, hydroxyl, C₃-C₆-cycloalkyl, C₁-C₄-alkoxy, C₁-C₄-alkylthio, di-(C₁-C₄-alkyl)amino, C₁-C₄-alkylcarbonyl, hydroxycarbonyl, C₁-C₄-alkoxycarbonyl, aminocarbonyl, C₁-C₄-alkylaminocarbonyl, di-(C₁-C₄-alkyl)-aminocarbonyl, or C₁-C₄-alkylcarbonyloxy;
   phenyl, phenyl-C₁-C₆-alkyl, phenylcarbonyl-C₁-C₆-alkyl,
   phenylsulfonylaminocarbonyl or phenyl-C₁-C₆-alkylcarbonyl,
   where the phenyl ring may be partially or fully halogenated and/or may carry one to three of the following groups: nitro, cyano, C₁-C₄-alkyl, C₁-C₄-haloalkyl, C₁-C₄-alkoxy or C₁-C₄-haloalkoxy; or
   SO₂R¹³
   particularly preferably hydrogen, C₁-C₆-alkyl, C₃-C₆-alkenyl, C₃-C₆-alkynyl, formyl, C₁-C₆-alkylcarbonyl, C₂-C₆-alkenylcarbonyl, C₁-C₆-alkoxycarbonyl, C₁-C₈-alkyl-sulfonylaminocarbonyl, di-(C₁-C₆-alkyl)aminocarbonyl, N—(C₁-C₆-alkoxy)-N—(C₁-C₆-alkyl)aminocarbonyl or di-(C₁-C₆-alkyl)aminothiocarbonyl,
   where the alkyl or alkoxy radicals mentioned may be partially or fully halogenated and/or may carry one to three of the following groups: cyano, C₁-C₄-alkoxy, C₁-C₄-alkoxycarbonyl, C₁-C₄-alkylaminocarbonyl, di-(C₁-C₄-alkyl)aminocarbonyl or C₁-C₄-alkylcarbonyloxy; phenyl-C₁-C₆-alkyl, phenylcarbonyl-C₁-C₆-alkyl, phenylsulfonyl-aminocarbonyl or phenyl-C₁-C₆-alkylcarbonyl,
   where the phenyl ring may be partially or fully halogenated and/or may carry one to three of the following groups: nitro, cyano, C₁-C₄-alkyl, C₁-C₄-haloalkyl, C₁-C₄-alkoxy or C₁-C₄-haloalkoxy; or
   SO₂R¹³;
   especially preferably hydrogen, C₁-C₆-alkyl, C₃-C₆-alkenyl, C₃-C₆-alkynyl, formyl, C₁-C₆-alkylcarbonyl, C₂-C₆-alkenylcarbonyl, C₁-C₆-alkoxycarbonyl, di-(C₁-C₆-alkyl)aminocarbonyl, N—(C₁-C₆-alkoxy)-N—(C₁-C₆-alkyl)aminocarbonyl, di-(C₁-C₆-alkyl)aminothiocarbonyl, phenyl-C₁-C₆-alkyl, phenylcarbonyl-C₁-C₆-alkyl or phenyl-C₁-C₆-alkylcarbonyl,
   where the phenyl ring may be partially or fully halogenated and/or may carry one to three of the following groups: nitro, cyano, C₁-C₄-alkyl, C₁-C₄-haloalkyl, C₁-C₄-alkoxy or C₁-C₄-halo-alkoxy; or SO₂R¹³.

Preference is also given to the benzoyl-substituted alanines of the formula I in which
R¹¹ is hydrogen, C₁-C₆-alkyl, C₃-C₆-alkenyl, C₃-C₆-alkynyl, formyl, C₁-C₆-alkylcarbonyl, C₂-C₆-alkenylcarbonyl, C₃-C₆-cycloalkylcarbonyl, C₁-C₆-alkoxycarbonyl, C₁-C₆-alkylaminocarbonyl, di-(C₁-C₆-alkyl)-aminocarbonyl, N—(C₁-C₆-alkoxy)-N—(C₁-C₆-alkyl)-aminocarbonyl, di-(C₁-C₆-alkyl)-aminothiocarbonyl, C₁-C₆-alkoxyimino-C₁-C₆-alkyl,
   where the alkyl, cycloalkyl or alkoxy radicals mentioned may be partially or fully halogenated and/or may carry one to three of the following groups: cyano, hydroxyl, C₃-C₆-cycloalkyl, C₁-C₄-alkoxy, C₁-C₄-alkylthio, di-(C₁-C₄-alkyl)amino, C₁-C₄-alkylcarbonyl, hydroxycarbonyl, C₁-C₄-alkoxycarbonyl, aminocarbonyl, C₁-C₄-alkylaminocarbonyl, di-(C₁-C₄-alkyl)aminocarbonyl or C₁-C₄-alkylcarbonyloxy; or
   SO₂R¹³.

Preference is also given to the benzoyl-substituted alanines of the formula I in which
R¹¹ is hydrogen, C₁-C₆-alkyl, C₃-C₆-alkenyl, C₃-C₆-alkynyl, formyl, C₁-C₆-alkylcarbonyl, C₁-C₆-alkoxycarbonyl, C₁-C₆-alkylaminocarbonyl, di-(C₁-C₆-alkyl)-aminocarbonyl, N—(C₁-C₆-alkoxy)-N—(C₁-C₆-alkyl)aminocarbonyl, especially preferably hydrogen or C₁-C₄-alkyl;
   where the alkyl and alkoxy radicals mentioned may be partially or fully halogenated and/or may carry one to three of the following groups: cyano, C₁-C₄-alkoxy, C₁-C₄-alkylaminocarbonyl or di-(C₁-C₄-alkyl)aminocarbonyl;
   phenyl-C₁-C₆-alkyl, phenylcarbonyl-C₁-C₆-alkyl, phenylaminocarbonyl or N—(C₁-C₆-alkyl)-N-(phenyl)aminocarbonyl,
   where the phenyl ring may be partially or fully halogenated and/or may carry one to three of the following groups: cyano, C₁-C₄-alkyl or C₁-C₄-haloalkyl; or
   SO₂R¹³;
   particularly preferably hydrogen, formyl, C₁-C₄-alkylcarbonyl, C₁-C₄-alkylamino-carbonyl, di-(C₁-C₄-alkyl)aminocarbonyl, phenylaminocarbonyl, N—(C₁-C₄-alkyl)-N-(phenyl)aminocarbonyl, SO₂CH₃, SO₂CF₃ or SO₂(C₆H₅).

Preference is also given to the benzoyl-substituted alanines of the formula I in which
R¹² is hydrogen, C₁-C₆-alkyl, hydroxyl or C₁-C₆-alkoxy;
   particularly preferably hydrogen or C₁-C₆-alkyl;
   especially preferably hydrogen or methyl;
   most preferably hydrogen.

Preference is also given to the benzoyl-substituted alanines of the formula I in which
R¹³ is C₁-C₆-alkyl, C₁-C₆-haloalkyl or phenyl,
   where the phenyl radical may be partially or fully halogenated and/or may be substituted by C₁-C₄-alkyl;
   particularly preferably C₁-C₄-alkyl, C₁-C₄-haloalkyl or phenyl;
   especially preferably methyl, trifluoromethyl or phenyl.

Preference is also given to the benzoyl-substituted alanines of the formula I in which
R¹³ is C₁-C₆-alkyl, C₁-C₆-haloalkyl or phenyl,
   where the phenyl radical may be partially halogenated and/or may be substituted by C₁-C₄-alkyl;
   particularly preferably C₁-C₄-alkyl, C₁-C₄-haloalkyl or phenyl;
   especially preferably methyl, trifluoromethyl or phenyl.

Particular preference is given to the benzoyl-substituted alanines of the formula I in which
R¹ is fluorine, chlorine or CF₃;
R² and R³, independently of one another, are hydrogen, fluorine or chlorine;
R⁴, R⁵, R⁶ and R⁷ are hydrogen;
R⁸ is C₁-C₄-alkyl,
   particularly preferably CH₃;
R⁹ is hydrogen;
R¹⁰ is hydrogen, C₁-C₆-alkyl, C₂-C₆-alkenyl, C₁-C₆-haloalkyl, C₂-C₆-haloalkenyl, C₁-C₆-hydroxyalkyl, hydroxycarbonyl-C₁-C₄-alkyl, phenyl-C₁-C₄-alkyl or phenyl-C₁-C₄-hydroxyalkyl; and
R¹¹ is hydrogen, formyl, C₁-C₄-alkylcarbonyl, C₁-C₄-alkylaminocarbonyl, di-(C₁-C₄-alkyl)aminocarbonyl, phenylaminocarbonyl, N—(C₁-C₄-alkyl)-N-(phenyl)-aminocarbonyl, SO₂CH₃, SO₂CF₃ or SO₂(C₆H₅);
R¹² is hydrogen.

Extraordinary preference is given to the compounds of the formula I.a (corresponds to formula I where R¹═CF₃, R², R³, R⁴, R⁵, R⁶, R⁷, R⁹ and R¹²═H; R⁸═CH₃), especially the compounds of the formulae I.a.1 to I.a.210 of Table 1, where the definitions of the variables R¹ to R¹² are of particular importance for the compounds according to the invention not only in combination with one another but in each case also on their own.

TABLE 1

| | |
|---|---|
| | I.a |
| 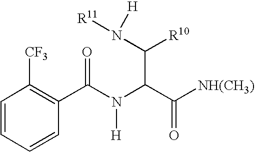 | |

| No. | R¹⁰ | R¹¹ |
|---|---|---|
| I.a.1 | H | H |
| I.a.2 | CH₃ | H |
| I.a.3 | CH═CH₂ | H |
| I.a.4 | CH═CH(CH₃) | H |
| I.a.5 | CH═C(CH₃)₂ | H |
| I.a.6 | C(CH₃)═CH₂ | H |
| I.a.7 | C(CH₃)═CH(CH₃) | H |
| I.a.8 | C≡CH | H |
| I.a.9 | C≡CCH₃ | H |
| I.a.10 | CF₃ | H |
| I.a.11 | CHF₂ | H |
| I.a.12 | CF₂CF₃ | H |
| I.a.13 | CH₂OH | H |
| I.a.14 | CH₂OCH₃ | H |
| I.a.15 | CH₂OC(O)CH₃ | H |
| I.a.16 | CH(OH)CH₂OH | H |
| I.a.17 | CH₂NHC(O)H | H |
| I.a.18 | CH₂NHC(O)CH₃ | H |
| I.a.19 | cyclopropyl | H |
| I.a.20 | cyclopentyl | H |
| I.a.21 | cyclohexyl | H |
| I.a.22 | cyclopenten-2-yl | H |
| I.a.23 | cyclohexen-2-yl | H |
| I.a.24 | tetrahydrofur-2-yl | H |
| I.a.25 | tetrahydrofur-3-yl | H |
| I.a.26 | tetrahydropyran-2-yl | H |
| I.a.27 | tetrahydropyran-3-yl | H |
| I.a.28 | tetrahydropyran-4-yl | H |
| I.a.29 | 3,6-dihydro-2H-pyran-4-yl | H |
| I.a.30 | 3,6-dihydro-2H-pyran-5-yl | H |
| I.a.31 | H | C(O)H |
| I.a.32 | CH₃ | C(O)H |
| I.a.33 | CH═CH₂ | C(O)H |
| I.a.34 | CH═CH(CH₃₎ | C(O)H |
| I.a.35 | CH═C(CH₃)₂ | C(O)H |
| I.a.36 | C(CH₃)═CH₂ | C(O)H |
| I.a.37 | C(CH₃)═CH(CH₃) | C(O)H |
| I.a.38 | C≡CH | C(O)H |
| I.a.39 | C≡CCH₃ | C(O)H |
| I.a.40 | CF₃ | C(O)H |
| I.a.41 | CHF₂ | C(O)H |
| i.a.42 | CF₂CF₃ | C(O)H |
| I.a.43 | CH₂OH | C(O)H |
| I.a.44 | CH₂OCH₃ | C(O)H |
| I.a.45 | CH₂OC(O)CH₃ | C(O)H |
| I.a.46 | CH(OH)CH₂OH | C(O)H |
| I.a.47 | CH₂NHC(O)H | C(O)H |
| I.a.48 | CH₂NHC(O)CH₃ | C(O)H |
| I.a.49 | cyclopropyl | C(O)H |
| I.a.50 | cyclopentyl | C(O)H |
| I.a.51 | cyclohexyl | C(O)H |
| I.a.52 | cyclopenten-2-yl | C(O)H |
| I.a.53 | cyclohexen-2-yl | C(O)H |
| I.a.54 | tetrahydrofur-2-yl | C(O)H |
| I.a.55 | tetrahydrofur-3-yl | C(O)H |
| I.a.56 | tetrahydropyran-2-yl | C(O)H |
| I.a.57 | tetrahydropyran-3-yl | C(O)H |
| I.a.58 | tetrahydropyran-4-yl | C(O)H |
| I.a.59 | 3,6-dihydro-2H-pyran-4-yl | C(O)H |
| I.a.60 | 3,6-dihydro-2H-pyran-5-yl | C(O)H |
| I.a.61 | H | C(O)CH₃ |
| I.a.62 | CH₃ | C(O)CH₃ |
| I.a.63 | CH═CH₂ | C(O)CH₃ |
| I.a.64 | CH═CH(CH₃) | C(O)CH₃ |
| I.a.65 | CH═C(CH₃)₂ | C(O)CH₃ |
| I.a.66 | C(CH₃)═CH₂ | C(O)CH₃ |
| I.a.67 | C(CH₃)═CH(CH₃) | C(O)CH₃ |
| I.a.68 | C≡CH | C(O)CH₃ |
| I.a.69 | C≡CCH₃ | C(O)CH₃ |
| I.a.70 | CF₃ | C(O)CH₃ |
| I.a.71 | CHF₂ | C(O)CH₃ |
| I.a.72 | CF₂CF₃ | C(O)CH₃ |
| I.a.73 | CH₂OH | C(O)CH₃ |
| I.a.74 | CH₂OCH₃ | C(O)CH₃ |
| I.a.75 | CH₂OC(O)CH₃ | C(O)CH₃ |
| I;a.76 | CH(OH)CH₂OH | C(O)CH₃ |
| I.a.77 | CH₂NHC(O)H | C(O)CH₃ |
| I.a.78 | CH₂NHC(O)CH₃ | C(O)CH₃ |
| I.a.79 | cyclopropyl | C(O)CH₃ |
| I.a.80 | cyclopentyl | C(O)CH₃ |
| I.a.81 | cyclohexyl | C(O)CH₃ |
| I.a.82 | cyclopenten-2-yl | C(O)CH₃ |
| I.a.83 | cyclohexen-2-yl | C(O)CH₃ |
| I.a.84 | tetrahydrofur-2-yl | C(O)CH₃ |
| I.a.85 | tetrahydrofur-3-yl | C(O)CH₃ |
| I.a.86 | tetrahydropyran-2-yl | C(O)CH₃ |
| I.a.87 | tetrahydropyran-3-yl | C(O)CH₃ |
| I.a.88 | tetrahydropyran-4-yl | C(O)CH₃ |
| I.a.89 | 3,6-dihydro-2H-pyran-4-yl | C(O)CH₃ |
| I.a.90 | 3,6-dihydro-2H-pyran-5-yl | C(O)CH₃ |
| I.a.91 | H | C(O)NH(CH₃) |
| I.a.92 | CH₃ | C(O)NH(CH₃) |
| I.a.93 | CH═CH₂ | C(O)NH(CH₃) |
| I.a.94 | CH═CH(CH₃) | C(O)NH(CH₃) |
| I.a.95 | CH═C(CH₃)₂ | C(O)NH(CH₃) |
| I.a.96 | C(CH₃)═CH₂ | C(O)NH(CH₃) |
| I.a.97 | C(CH₃)═CH(CH₃) | C(O)NH(CH₃) |
| I.a.98 | C≡CH | C(O)NH(CH₃) |
| I.a.99 | C≡CCH₃ | C(O)NH(CH₃) |
| I.a.100 | CF₃ | C(O)NH(CH₃) |
| I.a.101 | CHF₂ | C(O)NH(CH₃) |
| I.a.102 | CF₂CF₃ | C(O)NH(CH₃) |
| I.a.103 | CH₂OH | C(O)NH(CH₃) |
| I.a.104 | CH₂OCH₃ | C(O)NH(CH₃) |
| I.a.105 | CH₂OC(O)CH₃ | C(O)NH(CH₃) |
| I.a.106 | CH(OH)CH₂OH | C(O)NH(CH₃) |
| I.a.107 | CH₂NHC(O)H | C(O)NH(CH₃) |
| I.a.108 | CH₂NHC(O)CH₃ | C(O)NH(CH₃) |
| I.a.109 | cyclopropyl | C(O)NH(CH₃) |
| I.a.110 | cyclopentyl | C(O)NH(CH₃) |
| I.a.111 | cyclohexyl | C(O)NH(CH₃) |
| I.a.112 | cyclopenten-2-yl | C(O)NH(CH₃) |
| I.a.113 | cyclohexen-2-yl | C(O)NH(CH₃) |
| I.a.114 | tetrahydrofur-2-yl | C(O)NH(CH₃) |
| I.a.115 | tetrahydrofur-3-yl | C(O)NH(CH₃) |
| I.a.116 | tetrahydropyran-2-yl | C(O)NH(CH₃) |
| I.a.117 | tetrahydropyran-3-yl | C(O)NH(CH₃) |
| I.a.118 | tetrahydropyran-4-yl | C(O)NH(CH₃) |
| I.a.119 | 3,6-dihydro-2H-pyran-4-yl | C(O)NH(CH₃) |
| I.a.120 | 3,6-dihydro-2H-pyran-5-yl | C(O)NH(CH₃) |
| I.a.121 | H | C(O)N(CH₃)₂ |
| I.a.122 | CH₃ | C(O)N(CH₃)₂ |
| I.a.123 | CH═CH₂ | C(O)N(CH₃)₂ |
| I.a.124 | CH═CH(CH₃) | C(O)N(CH₃)₂ |
| I.a.125 | CH═C(CH₃)₂ | C(O)N(CH₃)₂ |
| I.a.126 | C(CH₃)═CH₂ | C(O)N(CH₃)₂ |
| I.a.127 | C(CH₃)═CH(CH₃) | C(O)N(CH₃)₂ |
| I.a.128 | C≡CH | C(O)N(CH₃)₂ |
| I.a.129 | C≡CCH₃ | C(O)N(CH₃)₂ |
| I.a.130 | CF₃ | C(O)N(CH₃)₂ |
| I.a.131 | CHF₂ | C(O)N(CH₃)₂ |
| I.a.132 | CF₂CF₃ | C(O)N(CH₃)₂ |
| I.a.133 | CH₂OH | C(O)N(CH₃)₂ |
| I.a.134 | CH₂OCH₃ | C(O)N(CH₃)₂ |
| I.a.135 | CH₂OC(O)CH₃ | C(O)N(CH₃)₂ |
| I.a.136 | CH(OH)CH₂OH | C(O)N(CH₃)₂ |
| I.a.137 | CH₂NHC(O)H | C(O)N(CH₃)₂ |
| I.a.138 | CH₂NHC(O)CH₃ | C(O)N(CH₃)₂ |
| I.a.139 | cyclopropyl | C(O)N(CH₃)₂ |
| I.a.140 | cyclopentyl | C(O)N(CH₃)₂ |
| I.a.141 | cyclohexyl | C(O)N(CH₃)₂ |
| I.a.142 | cyclopenten-2-yl | C(O)N(CH₃)₂ |
| I.a.143 | cyclohexen-2-yl | C(O)N(CH₃)₂ |
| I.a.144 | tetrahydrofur-2-yl | C(O)N(CH₃)₂ |
| I.a.145 | tetrahydrofur-3-yl | C(O)N(CH₃)₂ |
| I.a.146 | tetrahydropyran-2-yl | C(O)N(CH₃)₂ |
| I.a.147 | tetrahydropyran-3-yl | C(O)N(CH₃)₂ |
| I.a.148 | tetrahydropyran-4-yl | C(O)N(CH₃)₂ |
| I.a.149 | 3,6-dihydro-2H-pyran-4-yl | C(O)N(CH₃)₂ |
| I.a.150 | 3,6-dihydro-2H-pyran-5-yl | C(O)N(CH₃)₂ |
| I.a.151 | H | C(O)OCH₃ |
| I.a.152 | CH₃ | C(O)OCH₃ |
| I.a.153 | CH═CH₂ | C(O)OCH₃ |
| I.a.154 | CH═CH(CH₃) | C(O)OCH₃ |
| I.a.155 | CH═C(CH₃)₂ | C(O)OCH₃ |
| I.a.156 | C(CH₃)═CH₂ | C(O)OCH₃ |
| I.a.157 | C(CH₃)═CH(CH₃) | C(O)OCH₃ |
| I.a.158 | C≡CH | C(O)OCH₃ |
| I.a.159 | C≡CCH₃ | C(O)OCH₃ |
| I.a.160 | CF₃ | C(O)OCH₃ |
| I.a.161 | CHF₂ | C(O)OCH₃ |
| I.a.162 | CF₂CF₃ | C(O)OCH₃ |
| I.a.163 | CH₂OH | C(O)OCH₃ |
| I.a.164 | CH₂OCH₃ | C(O)OCH₃ |
| I.a.165 | CH₂OC(O)CH₃ | C(O)OCH₃ |
| I.a.166 | CH(OH)CH₂OH | C(O)OCH₃ |
| I.a.167 | CH₂NHC(O)H | C(O)OCH₃ |
| I.a.168 | CH₂NHC(O)CH₃ | C(O)OCH₃ |
| I.a.169 | cyclopropyl | C(O)OCH₃ |
| I.a.170 | cyclopentyl | C(O)OCH₃ |
| I.a.171 | cyclohexyl | C(O)OCH₃ |
| I.a.172 | cyclopenten-2-yl | C(O)OCH₃ |
| I.a.173 | cyclohexen-2-yl | C(O)OCH₃ |
| I.a.174 | tetrahydrofur-2-yl | C(O)OCH₃ |
| I.a.175 | tetrahydrofur-3-yl | C(O)OCH₃ |
| I.a.176 | tetrahydropyran-2-yl | C(O)OCH₃ |
| I.a.177 | tetrahydropyran-3-yl | C(O)OCH₃ |
| I.a.178 | tetrahydropyran-4-yl | C(O)OCH₃ |
| I.a.179 | 3,6-dihydro-2H-pyran-4-yl | C(O)OCH₃ |
| I.a.180 | 3,6-dihydro-2H-pyran-5-yl | C(O)OCH₃ |
| I.a.181 | H | SO₂CH₃ |
| I.a.182 | CH₃ | SO₂CH₃ |
| I.a.183 | CH═CH₂ | SO₂CH₃ |
| i.a.184 | CH═CH(CH₃) | SO₂CH₃ |
| I.a.185 | CH═C(CH₃)₂ | SO₂CH₃ |
| I.a.186 | C(CH₃)═CH₂ | SO₂CH₃ |
| I.a.187 | C(CH₃)═CH(CH₃) | SO₂CH₃ |
| I.a.188 | C≡CH | SO₂CH₃ |
| I.a.189 | C≡CCH₃ | SO₂CH₃ |
| I.a.190 | CF₃ | SO₂CH₃ |
| I.a.191 | CHF₂ | SO₂CH₃ |
| I.a.192 | CF₂CF₃ | SO₂CH₃ |
| I.a.193 | CH₂OH | SO₂CH₃ |
| I.a.194 | CH₂OCH₃ | SO₂CH₃ |
| I.a.195 | CH₂OC(O)CH₃ | SO₂CH₃ |
| I.a.196 | CH(OH)CH₂OH | SO₂CH₃ |
| I.a.197 | CH₂NHC(O)H | SO₂CH₃ |
| I.a.198 | CH₂NHC(O)CH₃ | SO₂CH₃ |
| I.a.199 | cyclopropyl | SO₂CH₃ |
| I.a.200 | cyclopentyl | SO₂CH₃ |
| I.a.201 | cyclohexyl | SO₂CH₃ |
| I.a.202 | cyclopenten-2-yl | SO₂CH₃ |
| I.a.203 | cyclohexen-2-yl | SO₂CH₃ |
| I.a.204 | tetrahydrofur-2-yl | SO₂CH₃ |
| I.a.205 | tetrahydrofur-3-yl | SO₂CH₃ |
| I.a.206 | tetrahydropyran-2-yl | SO₂CH₃ |
| I.a.207 | tetrahydropyran-3-yl | SO₂CH₃ |
| I.a.208 | tetrahydropyran-4-yl | SO₂CH₃ |
| I.a.209 | 3,6-dihydro-2H-pyran-4-yl | SO₂CH₃ |
| I.a.210 | 3,6-dihydro-2H-pyran-5-yl | SO₂CH₃ |

Most preference is also given to the compounds of the formula I.b, especially the compounds of the formulae I.b.1 to I.b.210 which differ from the corresponding compounds of the formulae I.a.1 to I.a.210 in that R² is fluorine:

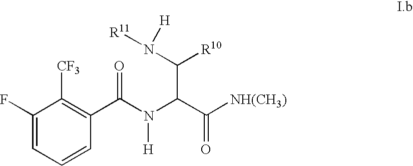

Most preference is also given to the compounds of the formula I.c, especially the compounds of the formula I.c.1 to I.c.210 which differ from the corresponding compounds of the formulae I.a.1 to I.a.210 in that R³ is fluorine:

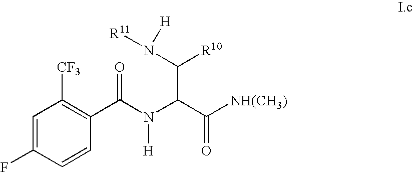

Most preference is also given to the compounds of the formula I.d, especially the compounds of the formula I.d.1 to i.d.210 which differ from the corresponding compounds of the formulae I.a.1 to I.a.210 in that R⁴ is fluorine:

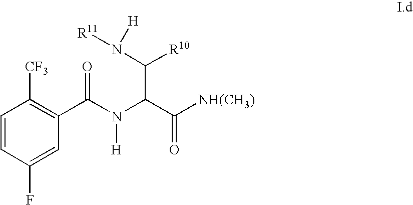

Most preference is also given to the compounds of the formula I.e, especially the compounds of the formula I.e.1 to I.e.210 which differ from the corresponding compounds of the formulae I.a.1 to I.a.210 in that R² is chlorine:

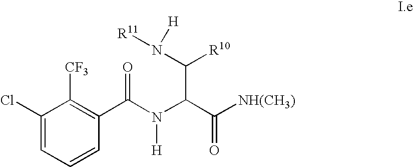

Most preference is also given to the compounds of the formula I.f, especially the compounds of the formula I.f.1 to I.f.210 which differ from the corresponding compounds of the formulae I.a.1 to I.a.210 in that R³ is chlorine:

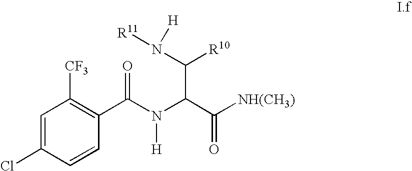

Most preference is also given to the compounds of the formula I.g, especially the compounds of the formula I.g.1 to I.g.210 which differ from the corresponding compounds of the formulae I.a.1 to I.a.210 in that R³ and R⁴ are fluorine:

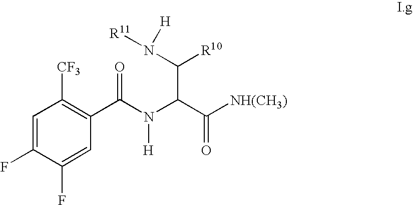

Most preference is also given to the compounds of the formula I.h, especially the compounds of the formula I.h.1 to I.h.210 which differ from the corresponding compounds of the formulae I.a.1 to I.a.210 in that R¹ is chlorine and R² is CF₃:

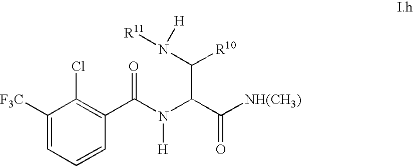

Most preference is also given to the compounds of the formula I.j, especially the compounds of the formula I.j.1 to I.j.210 which differ from the corresponding compounds of the formulae I.a.1 to I.a.210 in that R¹ and R² are chlorine:

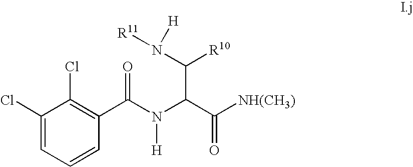

Most preference is also given to the compounds of the formula I.k, especially the compounds of the formula I.k.1 to I.k.210 which differ from the corresponding compounds of the formulae I.a.1 to I.a.210 in that R¹ and R³ are chlorine:

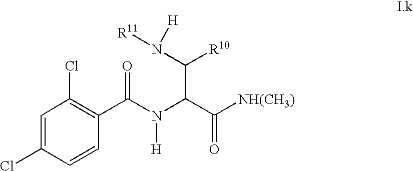

The benzoyl-substituted alanines of the formula I can be obtained by different routes, for example by the following processes:

### Process A

Alanine derivatives of the formula V are initially reacted with benzoic acids/benzoic acid derivatives of the formula IV to give the corresponding benzoyl derivatives of the formula III which then react with amines of the formula II to give the desired benzoyl-substituted alanines of the formula I:

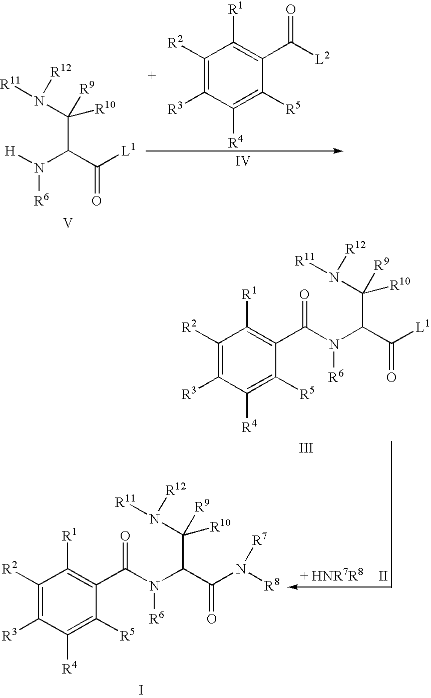

L¹ is a nucleophilically replaceable leaving group, for example hydroxyl or C₁-C₆-alkoxy.

L² is a nucleophilically replaceable leaving group, for example hydroxyl, halogen, C₁-C₆-alkylcarbonyl, C₁-C₆-alkoxycarbonyl, C₁-C₄-alkylsulfonyl, phosphoryl or isoureyl.

The reaction of the alanine derivatives of the formula V with benzoic acids/benzoic acid derivatives of the formula IV where L² is hydroxyl to give benzoyl derivatives of the formula III is carried out in the presence of an activating agent and a base, usually at temperatures of from 0° C. to the boiling point of the reaction mixture, preferably at from 0° C. to 110° C., particularly preferably at room temperature, in an inert organic solvent [cf. K. C. Nicolaou et al., J. of the Am. Chem. Soc. (2005), 127(31), 11176-11183; Shu-Sin Chng et al., Tetrahedron Lett. (2004), 45(52), 9501-9504; Werner W. K. R. Mederski et al., Bioorganic & Medicinal Chemistry Letters (2004), 14 (23), 5817-5822; Romano Silvestri et al., J. of Med. Chem. (2004), 47(15), 3892-3896; P. Rzepecki et al., J. of Org. Chem. (2004), 69(16), 5168-5178; Justin Bower et al., Bioorg. and Med. Chem. Lett. (2003), 13(15), 2455-2458; Jill Arrowsmith et al., J. of Med. Chem. (2002), 45(25), 5458-5470; Ashraf Briket al., Chemistry and Biology-(2002), 9(8), 891-896; Andrew D. Abell et al., Tetrahedron Lett. (2002), 43(20), 3673-3675; John F. Okonya et al., J. of Org. Chem. (2002), 67(4), 1102-1108; George R. Pettit et al., J. of Org. Chem. (1985), 50(15), 2654-2659].

Suitable activating agents are condensing agents, such as, for example, polystyrene-supported dicyclohexylcarbodiimide, diisopropylcarbodiimide, carbonyldiimidazole, chloroformates, such as methyl chloroformate, ethyl chloroformate, isopropyl chloroformate, isobutyl chloroformate, sec-butyl chloroformate or allyl chloroformate, pivaloyl chloride, polyphosphoric acid, propanephosphonic anhydride, bis(2-oxo-3-oxazolidinyl)-phosphoryl chloride (BOPCI) or sulfonyl chlorides, such as methanesulfonyl chloride, toluenesulfonyl chloride or benzenesulfonyl chloride.

Suitable solvents are aliphatic hydrocarbons, such as pentane, hexane, cyclohexane and mixtures of C₅-C₈-alkanes, aromatic hydrocarbons such as benzene, toluene, o-, m- and p-xylene, halogenated hydrocarbons, such as methylene chloride, chloroform and chlorobenzene, ethers, such as diethyl ether, diisopropyl ether, tert-butylmethyl ether, dioxane, anisole and tetrahydrofuran (THF), nitriles, such as acetonitrile and propionitrile, ketones, such as acetone, methyl ethyl ketone, diethyl ketone and tert-butyl methyl ketone, and also dimethyl sulfoxide, dimethylformamide (DMF), dimethylacetamide (DMA) and N-methylpyrrolidone (NMP) or else in water; particular preference is given to methylene chloride, THF and water.

It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate, and also alkali metal bicarbonates, such as sodium bicarbonate, moreover organic bases, for example tertiary amines, such as trimethylamine, triethylamine, diisopropylethylamine, N-methyl-morpholine, and N-methylpiperidine, pyridine, substituted pyridines, such as collidine, lutidine and 4-dimethylaminopyridine, and also bicyclic amines. Particular preference is given to sodium hydroxide, triethylamine and pyridine.

The bases are generally employed in equimolar amounts. However, they can also used in excess or, if appropriate, as solvents.

The starting materials are generally reacted with one another in equimolar amounts. It may be advantageous to employ an excess of IV, based on V.

The reaction mixtures are worked up in a customary manner, for example by mixing with water, separating the phases and, if appropriate, chromatographic purification of the crude products. Some of the intermediates are obtained in the form of viscous oils which may be purified or freed from volatile components under reduced pressure and at moderately elevated temperature. If the intermediates and end products are obtained as solids, purification may also be carried out by recrystallization or digestion. The reaction of the alanine derivatives of the formula V with benzoic acids/benzoic acid derivatives of the formula IV where L² is halogen, C₁-C₆-alkylcarbonyl, C₁-C₆-alkoxy-carbonyl, C₁-C₄-alkylsulfonyl, phosphoryl or isoureyl to give benzoyl derivatives of the formula III is carried out in the presence of a base, usually at temperatures of from 0° C. to the boiling point of the reaction mixture, preferably at from 0° C. to 100° C., particularly preferably at room temperature, in an inert organic solvent [cf. K. C. Nicolaou et al., J. of the Am. Chem. Soc. (2005), 127(31), 11176-11183; Shu-Sin Chng et al., Tetrahedron Lett. (2004), 45(52), 9501-9504; Werner W. K. R. Mederski et al., Bioorganic & Medicinal Chemistry Letters (2004), 14 (23), 5817-5822; Romano Silvestri et al., J. of Med. Chem. (2004), 47(15), 3892-3896; P. Rzepecki et al., J. of Org. Chem. (2004), 69(16), 5168-5178; Justin Bower et al., Bioorg. and Med. Chem. Lett. (2003), 13(15), 2455-2458; Jill Arrowsmith et al., J. of Med. Chem. (2002), 45(25), 5458-5470; Ashraf Briket al., Chemistry and Biology (2002), 9(8), 891-896; Andrew D. Abell et al., Tetrahedron Lett. (2002), 43(20), 3673-3675; John F. Okonya et al., J. of Org. Chem. (2002), 67(4), 1102-1108; George R. Pettit et al., J. of Org. Chem. (1985), 50(15), 2654-2659].

Suitable solvents are aliphatic hydrocarbons, such as pentane, hexane, cyclohexane and mixtures of C₅-C₈-alkanes, aromatic hydrocarbons such as benzene, toluene, o-, m- and p-xylene, halogenated hydrocarbons, such as methylene chloride, chloroform and chlorobenzene, ethers, such as diethyl ether, diisopropyl ether, tert-butylmethyl ether, dioxane, anisole and tetrahydrofuran (THF), nitriles, such as acetonitrile and propionitrile, ketones, such as acetone, methyl ethyl ketone, diethyl ketone and tert-butyl methyl ketone, and also dimethyl sulfoxide, dimethylformamide (DMF), dimethylacetamide (DMA) and N-methylpyrrolidone (NMP) or else in water; particular preference is given to methylene chloride, THF and water.

It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate, and also alkali metal bicarbonates, such as sodium bicarbonate, moreover organic bases, for example tertiary amines, such as trimethylamine, triethylamine, diisopropylethylamine, N-methylmorpholine, and N-methylpiperidine, pyridine, substituted pyridines, such as collidine, lutidine and 4-dimethylaminopyridine, and also bicyclic amines. Particular preference is given to sodium hydroxide, triethylamine and pyridine.

The bases are generally employed in equimolar amounts. However, they can also used in excess or, if appropriate, as solvents.

The starting materials are generally reacted with one another in equimolar amounts. It may be advantageous to employ an excess of IV, based on V.

Work-up and isolation of the products can be carried out in a manner known per se.

It is, of course, also possible to react initially, in an analogous manner, the alanine derivatives of the formula V with amines of the formula II to give the corresponding amides which are the reacted with benzoic acids/benzoic acid derivatives of the formula IV to give the desired benzoyl-substituted alanines of the formula I.

The alanine derivatives of the formula V (for example where L¹=hydroxyl or C₁-C₆-alkoxy) required for preparing the benzoyl derivatives of the formula III are, even in enantiomerically and diastereomerically pure form, known from the literature, or they can be prepared in accordance with the literature cited:
1. Condensation of the enolate of a glycine derivative with an imine derivative
[cf. Franklin A. Davis et al., Org. Lett. (2004), 6(14), 2397-2399; Alma Viso et al., J. of Org. Chem. (2004), 69(5), 1542-1547; Luca Bernardi et al., J. of Org. Chem. (2003), 68(7), 2583-2591; Ivanka K. Kavrakova et al, J. of Chem. Res., Synopses (1993), (5), 186-187.]
2. Reduction of an azide
[cf. Mark E. Bunnage et al., Organ. and Biomol. Chem. (2003), 1(21), 3708-3715; Anthony J. Burke et al., Synlett (1996), (7), 621-622.]
3. Nucleophilic opening of an aziridine
[cf. Michael A. Letavic et al., Bioorg. and Med. Chem. Lett. (2003), 13(19), 3243-3246; Tushar K. Chakraborty et al., Chem. Lett. (2003), 32(1), 82-83; K.-D. Lee et al., Tetrahedron (2001), 57(39), 8267-8276; Luciano Antolini et al., J. of Org. Chem. (1997), 62(25), 8784-8789; Johan Legters et al., Rec. des Trav. Chim. Pays-Bas (1992), 111(2), 59-68-]
4. Addition of nitroenolates to glycinimines and subsequent reduction:
[cf. Nagatoshi Nishiwaki et al., Angew. Chem., Int. Ed. (2001), 40(16), 2992-2995; Kristian Rahbek Knudsen et al., J. of the Am. Chem. Soc. (2001), 123(24), 5843-5844]
5. Hydrogenation of diaminoacrylates
[cf. Andrea J. Robinson et al., J. of Org. Chem. (2001), 66(12), 4148-4152; Ryoichi Kuwano et al., Tetrahedron Asym. (1998), 9(16), 2773-2775; Hiroyuki Setoi et al., Chem. and Pharm. Bull. (1989), 37(4), 1126-1127; Piedad Fernandez-Resa et al., J. of the Chem. Soc. Perkin Trans. I (1989), (1), 67-71]
6. Oxidative diamination of acrylates
[cf. Guigen L¹ et al., Tetrahedron Lett. (2000), 41(45), 8699-8703]
7. Opening of imidazolines
[cf. Tamio Hayashi et al., Tetrahedron Lett. (1996), 37(28), 4969-4972; Peter J. Dunn et al., J. of Org. Chem. (1990), 55(17), 5017-5025.]
8. Addition of an N-nucleophile to an aminoacrylate
[cf. B. Narasimhulu Naidu et al., J. of Org. Chem. (2003), 68(26); 10098-10102; Daeock Choi et al., Tetrahedron Lett. (1995), 36(41), 7371-7374; Montserrat Perez et al., Tetrahedron (1995), 51(30), 8355-8362; Rolf Meyer et al., Justus Liebigs Ann. Chem. (1977), (7), 1183-1193.]

The benzoic acids/benzoic acid derivatives of the formula IV required for preparing the benzoyl derivatives of the formula III are commercially available or can be prepared analogously to procedures known from the literature by means of a Grignard reaction from the corresponding halide [for example Chang-Ling Liu et al., J. of Fluorine Chem. (2004), 125(9), 1287-1290; Manfred Schlosser et al., Europ. J. of Org. Chem. (2002), (17), 2913-2920; Hoh-Gyu Hahn et al., Agricult. Chem. and Biotech. (English Edition) (2002), 45(1), 37-42; Jonatan 0 Smith et al., J. of Fluorine Chem. (1997), Vol. 1996-1997, 81(2), 123-128; Etsuji Okada et al., Heterocycles (1992), 34(4), 791-798; Aliyu B. Abubakar et al., J. of Fluorine Chem. (1991), 55(2), 189-198; J. Leroy, J of Fluorine Chem. (1991), 53(1), 61-70; Len F. Lee et al., J. of Heterocyclic Chem. (1990), 27(2), 243-245; Len F. Lee et al., J. of Heterocyclic Chem. (1985), 22(6), 1621-1630; Jacques Leroy et al., Synthesis (1982), (4), 313-315.]

The reaction of the benzoyl derivatives of the formula III where L¹=hydroxyl or salts thereof with amines of the formula II to give the desired benzoyl-substituted serineamides of the formula I is carried out in the presence of an activating agent and, if appropriate, in the presence of a base, usually at temperatures of from 0° C. to the boiling point of the reaction mixture, preferably at from 0° C. to 100° C., particularly preferably at room temperature, in an inert organic solvent. [cf. Perich, J. W., Johns, R. B., J. Org. Chem. 53 (17), 4103-4105 (1988); Somiai, C. et al., Synthesis (3), 285-287 (1992); Gupta, A. et al., J. Chem. Soc. Perkin Trans. 2, 1911 (1990); Guan et al., J. Comb. Chem. 2, 297 (2000)].

Suitable activating agents are condensing agents, such as, for example, polystyrene-supported dicyclohexylcarbodiimide, diisopropylcarbodiimide, carbonyldiimidazole, chloroformates, such as methyl chloroformate, ethyl chloroformate, isopropyl chloroformate, isobutyl chloroformate, sec-butyl chloroformate or allyl chloroformate, pivaloyl chloride, polyphosphoric acid, propanephosphonic anhydride, bis(2-oxo-3-oxazolidinyl)-phosphoryl chloride (BOPCI) or sulfonyl chlorides, such as methanesulfonyl chloride, toluenesulfonyl chloride or benzenesulfonyl chloride.

Suitable solvents are aliphatic hydrocarbons, such as pentane, hexane, cyclohexane and mixtures of C₅-C₈-alkanes, aromatic hydrocarbons such as benzene, toluene, o-, m- and p-xylene, halogenated hydrocarbons, such as methylene chloride, chloroform and chlorobenzene, ethers, such as diethyl ether, diisopropyl ether, tert-butylmethyl ether, dioxane, anisole and tetrahydrofuran (THF), nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol., and also dimethyl sulfoxide, dimethylformamide (DMF), dimethylacetamide (DMA) and N-methylpyrrolidone (NMP) or else in water; particular preference is given to methylene chloride, THF, methanol, ethanol and water.

It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate, and also alkali metal bicarbonates, such as sodium bicarbonate, moreover organic bases, for example tertiary amines, such as trimethylamine, triethylamine, diisopropylethylamine, N-methyl-morpholine, and N-methylpiperidine, pyridine, substituted pyridines, such as collidine, lutidine and 4-dimethylaminopyridine, and also bicyclic amines. Particular preference is given to sodium hydroxide, triethylamine, ethyldiisopropylamine, N-methylmorpholine and pyridine.

The bases are generally employed in catalytic amounts; however, they can also be employed in equimolar amounts, in excess or, if appropriate, as solvent.

The starting materials are generally reacted with one another in equimolar amounts. It may be advantageous to employ an excess of II, based on III.

Work-up and isolation of the products can be carried out in a manner known per se.

The reaction of the benzoyl derivatives of the formula III where L¹═C₁-C₆-alkoxy with amines of the formula II to give the desired benzoyl-substituted serineamides of the formula I is usually carried out at temperatures of from 0° C. to the boiling point of the reaction mixture, preferably at from 0° C. to 100° C., particularly preferably at room temperature, in an inert organic solvent, if appropriate in the presence of a base [cf. Kawahata, N. H. et al., Tetrahedron Lett. 43 (40), 7221-7223 (2002); Takahashi, K. et al., J. Org. Chem. 50 (18), 3414-3415 (1985); Lee, Y. et al., J. Am. Chem. Soc. 121 (36), 8407-8408 (1999)].

Suitable solvents are aliphatic hydrocarbons, such as pentane, hexane, cyclohexane and mixtures of C₅-C₈-alkanes, aromatic hydrocarbons such as benzene, toluene, o-, m- and p-xylene, halogenated hydrocarbons, such as methylene chloride, chloroform and chlorobenzene, ethers, such as diethyl ether, diisopropyl ether, tert-butylmethyl ether, dioxane, anisole and tetrahydrofuran (THF), nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide (DMF), dimethylacetamide (DMA) and N-methylpyrrolidone (NMP) or else in water; particular preference is given to methylene chloride, THF, methanol, ethanol and water.

It is also possible to use mixtures of the solvents mentioned.

The reaction can, if appropriate, be carried out in the presence of a base. Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate, and also alkali metal bicarbonates, such as sodium bicarbonate, moreover organic bases, for example tertiary amines, such as trimethylamine, triethylamine, diisopropylethylamine, N-methylmorpholine, and N-methylpiperidine, pyridine, substituted pyridines, such as collidine, lutidine and 4-dimethylaminopyridine, and also bicyclic amines. Particular preference is given to sodium hydroxide, triethylamine, ethyldiisopropylamine, N-methylmorpholine and pyridine.

The bases are generally employed in catalytic amounts; however, they can also be employed in equimolar amounts, in excess or, if appropriate, as solvent.

The starting materials are generally reacted with one another in equimolar amounts. It may be advantageous to employ an excess of II, based on III.

Work-up and isolation of the products can be carried out in a manner known per se. The amines of the formula II required for preparing the benzoyl-substituted alanines of the formula I are commercially available.

### Process B

Benzoyl derivatives of the formula III where R¹¹=R^{X} and R¹²= hydrogen can also be obtained by condensing acylated glycine derivatives of the formula VIII where the acyl group is a removable protective group such as benzyloxycarbonyl (cf. VIIIa where Σ= benzyl) or tert-butyloxycarbonyl (cf. VIIIa where v= tert-butyl) with imino compounds VII to give the corresponding aldol products VI where R¹¹=R^{X} and R¹²= hydrogen. The protective group is then removed, and the alanine derivative of the formula V formed in this manner where R¹¹=R^{X} and R¹²= hydrogen is acylated with benzoic acid/benzoic acid derivatives of the formula IV.

Analogously, it is also possible to react an acylated glycine derivative of the formula VIII where the acyl group is a substituted benzoyl radical (cf. VIIIb) in the presence of a base with an imino compound VII to give the benzoyl derivative III where R¹¹=R^{X} and R¹²= hydrogen:

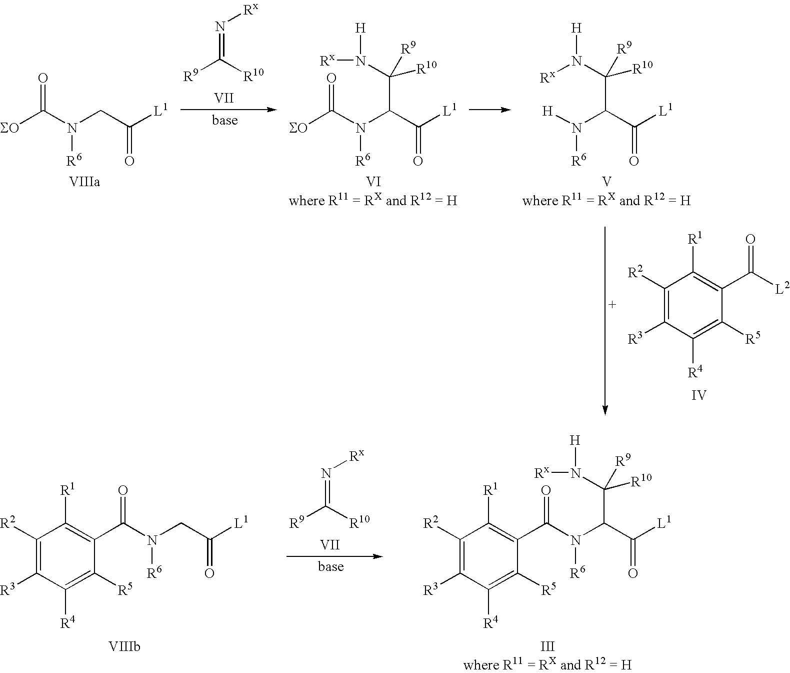

R^{X} is R¹¹ or a removable protective group such as C₁-C₆-alkyloxycarbonyl (for example tert-butyloxycarbonyl), C₁-C₆-alkylsulfinyl (for example tert-butylsulfinyl) or optionally C₁-C₆-alkyl-substituted arylsulfinyl (for example toluoylsulfinyl).

L¹ is a nucleophilically displaceable leaving group, for example hydroxyl or C₁-C₆-alkoxy.

L² is a nucleophilically displaceable leaving group, for example hydroxyl, halogen, C₁-C₆-alkylcarbonyl, C₁-C₆-alkoxycarbonyl, C₁-C₄-alkylsulfonyl, phosphoryl or isoureyl.

The reaction of the glycine derivatives VIII with imino compounds VII to give the corresponding aldol product VI where R¹¹=R^{X} and R¹²= hydrogen or the benzoyl derivative III where R¹¹=R^{X} and R¹²= hydrogen is usually carried out at temperatures of from −100° C. to the boiling point of the reaction mixture, preferably at from −80° C. to 20° C., especially preferably at from −80° C. to −20° C., in an inert organic solvent in the presence of a base [cf. J.-F. Rousseau et al., J. Org. Chem. 63, 2731-2737 (1998)].

Suitable solvents are aliphatic hydrocarbons, such as pentane, hexane, cyclohexane and mixtures of C₅-C₈-alkanes, aromatic hydrocarbons, such as toluene, o-, m- and p-xylene, ethers, such as diethyl ether, diisopropyl ether, tert-butyl methyl ether, dioxane, anisole and tetrahydrofuran, and also dimethyl sulfoxide, dimethylformamide and dimethylacetamide, particularly preferably diethyl ether, dioxane and tetrahydrofuran.

It is also possible to use mixtures of the solvents mentioned.

Suitable bases are, in general, inorganic compounds, such as alkali metal and alkaline earth metal hydride, such as lithium hydride, sodium hydride, potassium hydride and calcium hydride, alkali metal amides, such as lithium isopropylamide and lithium hexamethyldisilazide, organometallic compounds, in particular alkali metal alkyls, such as methyllithium, butyllithium and phenyllithium, and also alkali metal and alkaline earth metal alkoxides, such as sodium methoxide, sodium ethoxide, potassium ethoxide, potassium tert-butoxide, potassium-tert-pentoxide and dimethoxymagnesium, moreover organic bases, for example, tertiary amines, such as trimethylamine, triethylamine, diisopropylethylamine and N-methylpiperidine, pyridine, substituted pyridines, such as collidine, lutidine and 4-dimethylaminopyridine, and also bicyclic amines. Particular preference is given to sodium hydride, lithium hexamethyldisilazide and lithium diisopropylamide.

The bases are generally employed in equimolar amounts; however, they can also be employed in catalytic amounts, in excess or, if appropriate, as solvent.

The starting materials are generally reacted with one another in equimolar amounts. It may be advantageous to employ an excess of the base and/or the imino compounds VII, based on the glycine derivatives VIII.

Work-up and isolation of the products can be carried out in a manner known per se.

The glycine derivatives of the formula VIII required for preparing the benzoyl derivatives III where R¹¹=R^{X} and R¹²= hydrogen are commercially available, known from the literature [for example H. Pessoa-Mahana et al., Synth. Comm. 32, 1437 (2002)] or can be prepared in accordance with the literature cited.

The removal of the protective group Σ to give alanine derivatives of the formula V where R¹¹=R^{X} and R¹²= hydrogen is carried out by methods known from the literature [cf. J.-F. Rousseau et al., J. Org. Chem. 63, 2731-2737 (1998)); J. M. Andres, Tetrahedron 56, 1523 (2000)]; in the case of Σ= benzyl by hydrogenolysis, preferably using hydrogen and Pd/C in methanol; in the case of Σ= tert-butyl using acid, preferably using hydrochloric acid in dioxane.

The reaction of the alanine derivatives V where R¹¹=R^{X} and R¹²= hydrogen with benzoic acids/benzoic acid derivatives IV to give benzoyl derivatives III where R¹¹=Rx and R¹²= hydrogen is usually carried out analogously to the reaction, mentioned under process A, of the alanine derivatives of the formula V with benzoic acids/benzoic acid derivatives of the formula IV to give benzoyl derivatives Ill.

The removal, if R^{x} is a removable protective group, of this protective group R^{x}, which may be required, if appropriate, is carried out using methods known from the literature [cf. J.-F. Rousseau et al., J. Org. Chem. 63, 2731-2737 (1998)); J. M. Andres, Tetrahedron 56, 1523 (2000)]; in the case of Σ= benzyl by hydrogenolysis, preferably using hydrogen and Pd/C in methanol; in the case of Σ= tert-butyl using acid, preferably using hydrochloric acid in dioxane.

The benzoyl derivatives, obtainable in this manner, of the formula III where R¹¹ and R¹²= hydrogen can then be reacted with amines of the formula II analogously to process A to give the desired benzoyl-substituted alanines of the formula I where R¹¹ and R¹²= hydrogen, which can then be derivatized with compounds of the formula IX to give benzoyl-substituted alanines of the formula I where R¹²= hydrogen [cf., for example, Yokokawa, F. et al., Tetrahedron Lett. 42 (34), 5903-5908 (2001); Arrault, A. et al., Tetrahedron Lett. 43(22), 4041-4044 (2002)].

It is also possible to derivatize the benzoyl derivatives of the formula III where R¹¹ and R¹²= hydrogen initially with compounds of the formula IX to give further benzoyl derivatives of the formula III where R¹²= hydrogen [cf., for example, Jung-Hui Sun et al., Heterocycles (2004), 63(7), 585-1599; Christian Lherbet et al., Bioorg. and Med. Chem. Lett. (2003), 13(6), 997-1000; Masami Otsuka et al., Chem. and Pharm. Bull. (1985), 33(2), 509-514; J. R Piper et al., J. of Med. Chem. (1985), 28(8), 1016-1025] and then to react analogously to process A with amines of the formula II to give the desired benzoyl-substituted alanines of the formula I where R¹² hydrogen:

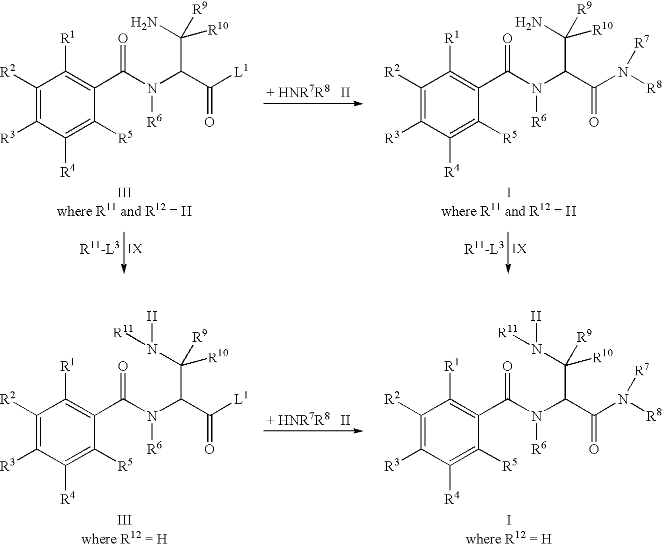

L¹ is a nucleophilically displaceable leaving group, for example hydroxyl or C₁-C₆-alkoxy.

L³ is a nucleophilically displaceable leaving group, for example halogen, hydroxyl or C₁-C₆-alkoxy.

The reaction of the benzoyl derivatives of the formula III (where R¹²= hydrogen and if appropriate where R¹¹= hydrogen) with amines of the formula II to give heteroaroyl-substituted alanines of the formula I (where R⁷= hydrogen and if appropriate where R⁶= hydrogen) usually takes place analogously to the reaction, described under Process A, of the benzoyl derivatives of the formula III with amines of the formula II.

The reaction of the benzoyl derivatives of the formula III where R¹¹ and R¹²= hydrogen or of the benzoyl-substituted alanines of the formula I where R¹¹ and R¹²= hydrogen with compounds of the formula IX to give benzoyl derivatives of the formula III where R¹²= hydrogen or benzoyl-substituted alanines of the formula I where R¹²= hydrogen is usually carried out at temperatures of from 0° C. to 100° C., preferably at from 10° C. to 50° C., in an inert organic solvent in the presence of a base [cf., for example, Jung-Hui Sun et al., Heterocycles (2004), 63(7), 585-1599; Christian Lherbet et al., Bioorg. and Med. Chem. Lett. (2003), 13(6), 997-1000; Masami Otsuka et al., Chem. and Pharm. Bull. (1985), 33(2), 509-514; J. R Piper et al., J. of Med. Chem. (1985), 28(8), 1016-1025].

Suitable solvents are aliphatic hydrocarbons, such as pentane, hexane, cyclohexane and mixtures of C₅-C₈-alkanes, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butylmethyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide, particularly preferably dichloromethane, tert-butyl methyl ether, dioxane and tetrahydrofuran.

It is also possible to use mixtures of the solvents mentioned.

Suitable bases are in general inorganic compounds such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metall amides, such as lithium amide, sodium amide and potassium amide, alkali metal and alkaline earth metal carbonates, such as lithium carbonate, potassium carbonate and calcium carbonate and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide, potassium tert-pentoxide and dimethoxymagnesium, auβerdem organic bases, for example, tertiary amines, such as trimethylamine, triethylamine, diisopropylethylamine and N-methylpiperidine, pyridine, substituted pyridines, such as collidine, lutidine and 4-dimethylaminopyridine, and also bicyclic amines. Particular preference is given to sodium hydroxide, sodium hydride and triethylamine.

The bases are generally employed in equilmolar amounts; however, they can also be employed in catalytic amounts, in excess or, if appropriate, as solvent.

The starting materials are generally reacted with one another in equimolar amounts. It may be advantageous to employ an excess of base and/or IX, based on III or 1.

Work-up and isolation of the products can be carried out in a manner known per se.

### Process C

Benzoyl derivatives of the formula III where R¹¹ and R¹²= hydrogen can also be obtained by reacting glycine derivatives of the formula XII with a nitro compound of the formula XI to give nitroaniline derivatives of the formula X, followed by reduction:

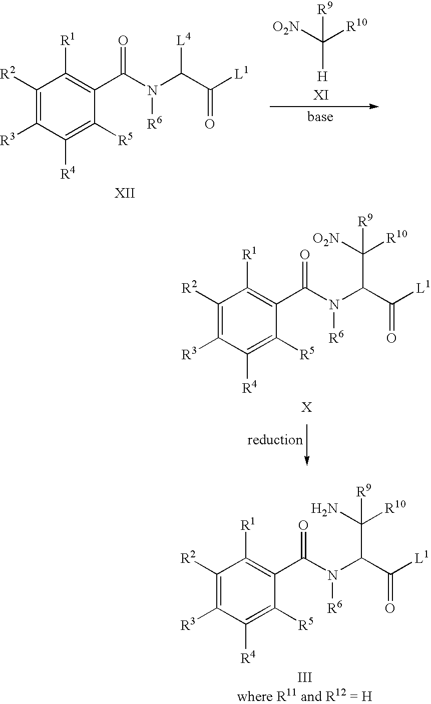

L¹ is a nucleophilically displaceable leaving group, for example hydroxyl or C₁-C₆-alkoxy.

L⁴ is a nucleophilically displaceable leaving group, for example halogen, such as chlorine or bromine.

The reaction of the nitro compound XI with the glycine derivative XII is usually carried out at a temperature of from −100° C. to the boiling point of the reaction mixture, preferably at from −80° C. to 20° C., in an inert organic solvent in the presence of a base (cf. Vicky A. Burgess et al., Aust. J. of Chem. (1988), 41(7), 1063-1070).

Suitable solvents are aliphatic hydrocarbons, such as pentane, hexane, cyclohexane and mixtures of C₅-C₈-alkanes, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butylmethyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide, particularly preferably dichloromethane, tert-butyl methyl ether, dioxane and tetrahydrofuran.

It is also possible to use mixtures of the solvents mentioned.

Suitable bases are in general inorganic compounds such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metall amides, such as lithium amide, sodium amide and potassium amide, alkali metal and alkaline earth metal carbonates, such as lithium carbonate, potassium carbonate and calcium carbonate and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide, potassium tert-pentoxide and dimethoxymagnesium, auerdem organic bases, for example, tertiary amines, such as trimethylamine, triethylamine, diisopropylethylamine and N-methylpiperidine, pyridine, substituted pyridines, such as collidine, lutidine and 4-dimethylaminopyridine, and asIo bicyclic amines. Particular preference is given to sodium hydroxide, sodium hydride and triethylamine.

The bases are generally employed in equimolar amounts; however, they can also be employed in catalytic amounts, in excess or, if appropriate, as solvent.

The starting materials are generally reacted with one another in equimolar amounts. It may be advantageous to employ an excess of base and/or XI, based on XII.

Work-up and isolation of the products can be carried out in a manner known per se.

The required nitro compounds of the formula XI are commercially available.

The required glycine derivatives of the formula XII can be obtained analogously to methods known from the literature (cf. Vicky A. Burgess et al., Aust. J. of Chem. (1988), 41(7), 1063-1070).

The reduction of the nitroaniline derivatives of the formula X is usually carried out at a temperature of from −100° C. to the boiling point of the reaction mixture, preferably at from −80° C. to 20° C., in an inert organic solvent using a reducing agent (cf. Vicky A. Burgess et al., Aust. J. of Chem. (1988), 41(7), 1063-1070).

Suitable solvents are aliphatic hydrocarbons, such as pentane, hexane, cyclohexane and mixtures of C₅-C₈-alkanes, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butylmethyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide, particularly preferably toluene, methylene chloride or tert-butyl methyl ether.

Suitable reducing agents are transition metal catalysts (for example Pd/C or Raney-Ni) in combination with hydrogen.

Work-up and isolation of the products can be carried out in a manner known per se.

Benzoyl derivatives of the formula III

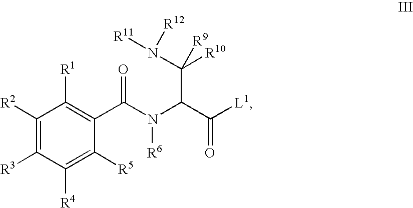

where R¹ to R⁶ and also R⁹ to R¹² are as defined above and L¹ is a nucleophilically replaceable leaving group, for example hydroxyl or C₁-C₆-alkoxy, are also provided by the invention.

The particularly preferred embodiments of the intermediates with respect to the variables correspond to those of the radicals R¹ to R⁶ and also R⁹ to R¹² of the formula Particular preference is given to heteroaroyl derivatives of the formula III in which
R¹ is fluorine, chlorine or CF₃;
R² and R³, independently of one another, are hydrogen, fluorine or chlorine;
R⁴, R⁵ and R⁶ are hydrogen;
R⁹ is hydrogen;
R¹⁰ is hydrogen, C₁-C₆-alkyl, C₂-C₆-alkenyl, C₁-C₆-haloalkyl, C₂-C₆-haloalkenyl, C₁-C₆-hydroxyalkyl, hydroxycarbonyl-C₁-C₄-alkyl, phenyl-C₁-C₄-alkyl or phenyl-C₁-C₄-hydroxyalkyl;
R¹¹ is hydrogen, formyl, C₁-C₄-alkylcarbonyl, C₁-C₄-alkylaminocarbonyl, Di-(C₁-C₄-alkyl)-aminocarbonyl, phenylaminocarbonyl, N—(C₁-C₄-alkyl)-N-(phenyl)-aminocarbonyl, SO₂CH₃, SO₂CF₃ or SO₂(C₆H₅); and
R¹² is hydrogen.

The following examples serve to illustrate the invention.

### PREPARATION EXAMPLES

### Example 1

### N-(2-Amino-3,3,3-trifluoro-1-methylcarbamoylpropyl)-4-fluoro-2-trifluoromethyl-benzamide (No. 3.4)

### 1.1) N-(Bromomethylcarbamoylmethyl)-4-fluoro-2-trifluoromethylbenzamide

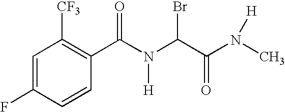

With irradiation (250 W incandescent lamp) and under reflux, 2.6 g (0.016 mol) of bromine were added dropwise to a suspension of 5 g (0.018 mol) of 4-fluoro-N methylcarbamoylmethyl-2-trifluoromethylebenzamide in 250 ml of CCl₄. The mixture was irradiated under reflux for a further 30 min and then cooled, and the solvent was removed under reduced pressure. This gave the title compound (6.6 g of crude product) which was used without further purification for the next step. ¹H-NMR (CDCl₃): =2.90 (d, 3H), 6.45 (m, 1H), 6.65 (d, 1H), 7.35 (m, 1H), 7.45 (m, 1H), 7.60 (m, 2H).

### 1.2) 4-Fluoro-2-trifluoromethyl-A-(3,3,3-trifluoro-1-methylcarbamoyl-2-nitropropyl)-benzamide

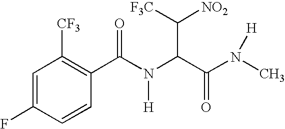

At −78° C., 7.2 g (0.017 mol) of n-butyllithium (15% in hexane) were added dropwise to a solution of 2.2 g (0.017 mol) of 1,1,1-trifluoro-2-nitroethane and 0.5 g (0.004 mol) of DMPU in 50 ml of THF, and the mixture was stirred at 0° C. for 30 min. At −78° C., a solution of 2 g (0.056 mol) of N-(bromomethylcarbamoylmethyl)-4-fluoro-2-trifluoro-methylbenzamide in 5 ml of THF was then added dropwise, and the mixture was stirred at −78° C. for another 4 h. Using dilute HCl, the pH was adjusted to 1, and the mixture was extracted with ethyl acetate. The combined organic phases were dried over Na₂SO₄, concentrated under reduced pressure and purified chromatographically on silica gel (cyclohexane/ethyl acetate 4:1). The product was stirred again with diethyl ether, filtered off and dried. This gave 0.45 g (20% of theory) of the title compound (diastereomer ratio 1:1) as a colorless solid of m.p. 224° C.

¹H-NMR (CDCl₃, diastereomer ratio 1:1): δ=2.75 (d), 2.80 (d) [3H], 5.55 (dd), 5.65 (dd) [I H], 5.85 (m), 5.95 (m) [1H], 7.35 (m, 1H), 7.45 (m, 1H), 7.55 (m, 1H), 7.85 (m), 8.10 (m) [1H], 9.05 (d), 9.20 (d) [1H].

### 1.3) N-(2-Amino-3,3,3-trifluoro-1-methylcarbamoylpropyl)-4-fluoro-2-trifluoromethyl-benzamide (No. 3.4)

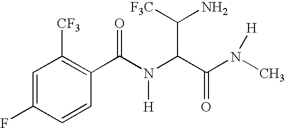

1.0 g of Raney Nickel (moist) was added to a solution of 1.0 g (2.47 mmol) of 4-fluoro-2-trifluoromethyl-N-(3,3,3-trifluoro-1-methylcarbamoyl-2-nitropropyl)benzamide in 70 ml of methanol, and the mixture was stirred at room temperature under a slightly superatmospheric hydrogen pressure for 16 h. The mixture was then flushed with nitrogen and filtered through Na₂SO₄, and the solvent was removed under reduced pressure. This gave 0.84 g (91% of theory) of the title compound (diastereomer mixture) as a colorless solid.

¹H-NMR (d₄-MeOH, diastereomer mixture): δ=2.80 (d, 3H), 3.70 (m), 3.90 (m) [1H], 4.80 (d), 5.00 (d) [1H], 7.45 (m, 1H), 7.55 (m, 1H), 7.70 (m, 1H).

### Example 2

### N-(2-Acetylamino-3,3,3-trifluoro-1-methylcarbamoylpropyl)-4-fluoro-2-trifluoromethyl-benzamide (No. 3.6)

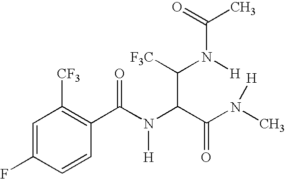

49 mg (0.48 mmol of triethylamine and 30 mg (0.38 mmol) of acetyl chloride were added to a solution of 120 mg (0.32 mmol) of A/(2-amino-3,3,3-trifluoro-1-methyl-carbamoylpropyl)-4-fluoro-2-trifluoromethylbenzamide in 5 ml of CH₂Cl₂, and the mixture was stirred at room temperature for 16 h. The reaction mixture was washed with dilute hydrochloric acid and saturated NaHCO₃ solution, dried over Na₂SO₄ and concentrated under reduced pressure. 50 mg (37% of theory) of the title compound were isolated as a colorless solid.

LC-MS (ESI): 440 [M+Na]⁺, 418 [M+H]⁺.

In addition to the compounds above, Tables 2 and 3 below list further benzoyl derivatives of the formula III and also benzoyl-substituted alanines of the formula I which were prepared or are preparable in a manner analogous to the processes described above.

TABLE 2

| | |
|---|---|
| | III |
| 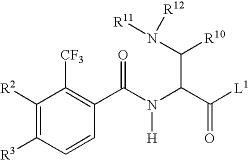 | |

where R¹ = CF₃, R⁴, R⁵, R⁶, R⁹ = H

| | | | | | | | Diastereomer | | m.p. | M⁺ + H |
|---|---|---|---|---|---|---|---|---|---|---|
| No. | R² | R³ | R¹⁰ | R¹¹ | R¹² | L¹ | ratio | Chirality | [° C.] | (m/z) |
| 2.1. | H | H | H | H | H | OH | — | 2-S | | 277 |
| 2.2. | H | H | H | SO₂-2-thienyl | H | OCH₃ | — | 2-S | | 437 |
| 2.3. | F | H | H | H | H | OH | — | 2-S | | 295 |
| 2.4. | H | F | H | H | H | OH | — | 2-S | | 295 |
| 2.5. | H | F | H | C(O)NH(CH₂CH₂CH₃) | H | OH | — | 2-S | | 380 |

TABLE 3

| | |
|---|---|
| | I |
| 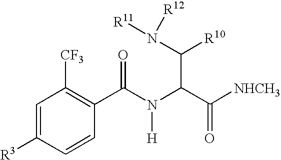 | |

where R¹ = CF₃, R², R⁴, R⁵, R⁶, R⁷, R⁹ = H; R⁸ = CH₃

| | | | | | | | | |
|---|---|---|---|---|---|---|---|---|
| | | | | | Diastereomer | | m.p. | M⁺ + H |
| No. | R³ | R¹⁰ | R¹¹ | R¹² | ratio | Chirality | [° C.] | (m/z) |

| | | | | | | | | |
|---|---|---|---|---|---|---|---|---|
| 3.1. | F | H | CH₃ | H | — | rac | 172 | |
| 3.2. | F | H | C(O)OC(CH₃)₃ | H | — | 2-S | 193 | |
| 3.3. | F | CH₃ | CH₃ | H | 1:0 | rac | 137 | |
| 3.4. | F | CF₃ | H | H | 1:1 | rac | | 376 |
| 3.5. | F | CF₃ | CH₃ | H | 4:1 | rac | 200 | |
| 3.6. | F | CF₃ | COCH₃ | H | 1:1 | rac | | 418 |
| 3.7. | F | CF₃ | COC(CH₃)₃ | H | 1:1 | rac | 244 | |
| 3.8. | F | CF₃ | CON(CH₃)₂ | H | 1:1 | rac | | 447 |
| 3.9. | F | CF₃ | C(O)NHC(O)OCH₂CH₃ | H | 1:1 | rac | | 491 |
| 3.10. | F | CF₃ | C(O)NH(SO₂)(4-CH₃—C₆H₄) | H | 1:1 | rac | 210 | |
| 3.11. | F | C(O)NH(CH₃) | CH₃ | H | 1:1 | rac | | 379 |
| 3.12. | F | CH₂OH | CH₃ | H | 7:3 | rac | 141 | |
| 3.13. | F | CH₂C₅H₅ | H | H | 1:0 | rac | 191 | |
| 3.14. | F | CH₂C₆H₅ | CH₃ | H | 4:1 | rac | 190 | |
| 3.15. | F | CH₂C₆H₅ | CH₃ | H | 1:0 | rac | 111 | |
| 3.16. | F | CH₂C₆H₅ | COCH₃ | H | 2:1 | rac | | 440 |
| 3.17. | F | CH₂C₆H₅ | COCH₃ | H | 1:2 | rac | | 440 |
| 3.18. | F | CH₂C₆H₅ | COCH₃ | CH₃ | 4:1 | rac | 103 | |
| 3.19. | F | CH₂C₆H₅ | COC(CH₃)₃ | CH₃ | 4:1 | rac | | 496 |
| 3.20. | F | CH₂C₆H₅ | CON(CH₃)₂ | H | 2:1 | rac | | 469 |
| 3.21. | F | CH₂C₆H₅ | CON(CH₃)₂ | CH₃ | 4:1 | rac | | 483 |
| 3.22. | F | CH₂C₆H₅ | SO₂CH₃ | H | 2:1 | rac | | 476 |
| 3.23. | F | CH₂C₆H₅ | SO₂CH₃ | CH₃ | 4:1 | rac | | 490 |
| 3.24. | F | 2-Cl-3-pyridyl | CH₃ | H | 1:1 | rac | 224 | |
| 3.25. | F | 2-Cl-3-pyridyl | COCH₃ | CH₃ | 1:1 | rac | 226 | |
| 3.26. | F | 2-Cl-3-pyridyl | SO₂CH₃ | CH₃ | 1:1 | rac | | 510 |
| 3.27. | F | 2-OH-3-indolyl | CH₃ | H | 3:2 | rac | | 453 |
| 3.28. | H | CH₂C₆H₅ | CH₃ | H | 1:1 | | 149 | |
| 3.29. | H | CH₂C₆H₅ | COCH₃ | CH₃ | 1:1 | rac | 193 | |
| 3.30. | H | CH₂C₆H₅ | COC(CH₃)₃ | CH₃ | 1:0 | rac | 183 | |
| 3.31. | H | CH₂C₆H₅ | COC(CH₃)₃ | CH₃ | 0:1 | rac | | 478 |
| 3.32. | H | CH₂C₆H₅ | CON(CH₃)₂ | CH₃ | 1:1 | rac | 77 | |
| 3.33. | H | CH₂C₆H₅ | SO₂CH₃ | CH₃ | 1:1 | rac | 223 | |
| 3.34. | H | H | C(O)OC(CH₃)₃ | H | — | 2-S | 196 | |
| 3.35. | H | H | C(O)-2-thienyl | H | — | 2-S | | 400 |
| 3.36. | H | H | H | H | — | 2-S | 154 | |
| 3.37. | H | H | C(S)NH(C₂H₅) | H | — | 2-S | | 377 |
| 3.38. | H | H | C(S)NH(CH₂CH═CH₂) | H | — | 2-S | | 389 |
| 3.39. | H | H | C(S)NH[CH₂CH(CH₃)₂] | H | — | 2-S | | 405 |
| 3.40. | H | H | C(S)NH[C(CH₃)₃] | H | — | | | 405 |
| 3.41. | H | H | C(O)NHCH(CH₃)₂ | H | — | 2-S | | 375 |
| 3.42. | H | H | C(O)NH(CH₂CH₂CH₃) | H | — | 2-S | | 375 |
| 3.43. | H | H | C(S)NH₂ | H | — | 2-S | | 349 |
| 3.44. | H | H | C(O)N(CH₃)₂ | H | — | 2-S | 152 | |
| 3.45. | F | 2-Cl-C₆H₄ | C(O)N(CH₃)₂ | H | 3:1 | rac | 187 | 491 |
| 3.46. | F | 2-CH₃-3-F-C₆H₃ | C(O)N(CH₃)₂ | H | 2:1 | rac | 180 | 487 |

### Biological Activity

The benzoyl-substituted alanines of the formula I and their agriculturally useful salts are suitable, both in the form of isomer mixtures and in the form of the pure isomers, as herbicides. The herbicidal compositions comprising compounds of the formula I control vegetation on non-crop areas very efficiently, especially at high rates of application. They act against broad-leaved weeds and grass weeds in crops such as wheat, rice, maize, soya and cotton without causing any significant damage to the crop plants. This effect is mainly observed at low rates of application.

Depending on the application method in question, the compounds of the formula I, or herbicidal compositions comprising them, can additionally be employed in a further number of crop plants for eliminating undesirable plants. Examples of suitable crops are the following:

Allium cepa, Ananas comosus, Arachis hypogaea, Asparagus officinalis, Beta vulgaris spec. altissima, Beta vulgaris spec. rapa, Brassica napus var. napus, Brassica napus var. napobrassica, Brassica rapa var. silvestris, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pisum sativum, Prunus avium, Prunus persica, Pyrus communis, Ribes sylvestre, Ricinus communis, Saccharum officinarum, Secale cereale, Solanum tuberosum, Sorghum bicolor (s. vulgare), Theobroma cacao, Trifolium pratense, Triticum aestivum, Triticum durum, Vicia faba, Vitis vinifera and Zea mays.

In addition, the compounds of the formula I may also be used in crops which tolerate the action of herbicides owing to breeding, including genetic engineering methods.

In addition, the compounds of the formula I may also be used in crops which tolerate attack by fungi or insects owing to breeding, including genetic engineering methods.

The compounds of the formula I, or the herbicidal compositions comprising them, can be used for example in the form of ready-to-spray aqueous solutions, powders, suspensions, also highly concentrated aqueous, oily or other suspensions or dispersions, emulsions, oil dispersions, pastes, dusts, materials for broadcasting, or granules, by means of spraying, atomizing, dusting, spreading or watering. The use forms depend on the intended purpose; in any case, they should ensure the finest possible distribution of the active ingredients according to the invention.

The herbicidal compositions comprise a herbicidally effective amount of at least one compound of the formula I or an agriculturally useful salt of 1, and auxiliaries which are customary for the formulation of crop protection agents.

Suitable as inert auxiliaries are essentially the following: mineral oil fractions of medium to high boiling point, such as kerosene and diesel oil, furthermore coal tar oils and oils of vegetable or animal origin, aliphatic, cyclic and aromatic hydrocarbons, for example paraffin, tetrahydronaphthalene, alkylated naphthalenes and their derivatives, alkylated benzenes and their derivatives, alcohols such as methanol, ethanol, propanol, butanol and cyclohexanol, ketones such as cyclohexanone, strongly polar solvents, for example amines such as N-methylpyrrolidone, and water.

Aqueous use forms can be prepared from emulsion concentrates, suspensions, pastes, wettable powders or water-dispersible granules by adding water. To prepare emulsions, pastes or oil dispersions, the substrates, either as such or dissolved in an oil or solvent, can be homogenized in water by means of a wetting agent, tackifier, dispersant or emulsifier. Alternatively, it is also possible to prepare concentrates comprising active substance, wetting agent, tackifier, dispersant or emulsifier and, if desired, solvent or oil, which are suitable for dilution with water.

Suitable surfactants (adjuvants) are the alkali metal salts, alkaline earth metal salts and ammonium salts of aromatic sulfonic acids, for example ligno-, phenol-, naphthalene- and dibutylnaphthalenesulfonic acid, and of fatty acids, alkyl- and alkylarylsulfonates, alkyl sulfates, lauryl ether sulfates and fatty alcohol sulfates, and salts of sulfated hexa-, hepta- and octadecanols, and also of fatty alcohol glycol ethers; condensates of sulfonated naphthalene and its derivatives with formaldehyde, condensates of naphthalene or of the naphthalenesulfdnic acids with phenol and formaldehyde, polyoxyethylene octylphenol ether, ethoxylated isooctyl-, octyl- or nonylphenol, alkylphenyl or tributylphenyl polyglycol ether, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignosulfite waste liquors or methylcellulose.

Powders, materials for broadcasting and dusts can be prepared by mixing or grinding the active ingredients together with a solid carrier.

Granules, for example coated granules, impregnated granules and homogeneous granules, can be prepared by binding the active ingredients to solid carriers. Solid carriers are mineral earths such as silicas, silica gels, silicates, talc, kaolin, limestone, lime, chalk, bole, loess, clay, dolomite, diatomaceous earth, calcium sulfate, magnesium sulfate and magnesium oxide, ground synthetic materials, fertilizers such as ammonium sulfate, ammonium phosphate, ammonium nitrate and ureas, al id products of vegetable origin, such as cereal meal, tree bark meal, wood meal and nutshell meal, cellulose powders, or other solid carriers.

The concentrations of the compounds of the formula I in the ready-to-use preparations can be varied within wide ranges. In general, the formulations comprise approximately from 0.001 to 98% by weight, preferably 0.01 to 95% by weight of at least one active ingredient. The active ingredients are employed in a purity of from 90% to 100%, preferably 95% to 100% (according to NMR spectrum). The formulation examples below illustrate the preparation of such preparations:
I. 20 parts by weight of an active compound of the formula I are dissolved in a mixture composed of 80 parts by weight of alkylated benzene, 10 parts by weight of the adduct of from 8 to 10 mol of ethylene oxide to 1 mol of oleic acid N-monoethanolamide, 5 parts by weight of calcium dodecylbenzenesulfonate and 5 parts by weight of the adduct of 40 mol of ethylene oxide to 1 mol of castor oil. Pouring the solution into 100 000 parts by weight of water and finely distributing it therein gives an aqueous dispersion which comprises 0.02% by weight of the active ingredient of formula I.
II. 20 parts by weight of an active compound of the formula I are dissolved in a mixture composed of 40 parts by weight of cyclohexanone, 30 parts by weight of isobutanol, 20 parts by weight of the adduct of 7 mol of ethylene oxide to 1 mol of isooctylphenol and 10 parts by weight of the adduct of 40 mol of ethylene oxide to 1 mol of castor oil. Pouring the solution into 100 000 parts by weight of water and finely distributing it therein gives an aqueous dispersion which comprises 0.02% by weight of the active ingredient of formula I.
III. 20 parts by weight of an active compound of the formula I are dissolved in a mixture composed of 25 parts by weight of cyclohexanone, 65 parts by weight of a mineral oil fraction of boiling point 210 to 280° C. and 10 parts by weight of the adduct of 40 mol of ethylene oxide to 1 mol of castor oil. Pouring the solution into 100 000 parts by weight of water and finely distributing it therein gives an aqueous dispersion which comprises 0.02% by weight of the active ingredient of formula I.
IV. 20 parts by weight of an active compound of the formula I are mixed thoroughly with 3 parts by weight of sodium diisobutylnaphthalenesulfonate, 17 parts by weight of the sodium salt of a lignosulfonic acid from a sulfite waste liquor and 60 parts by weight of pulverulent silica gel, and the mixture is ground in a hammer mill. Finely distributing the mixture in 20 000 parts by weight of water gives a spray mixture which comprises 0.1% by weight of the active ingredient of formula
V. 3 parts by weight of an active compound of the formula I are mixed with 97 parts by weight of finely divided kaolin. This gives a dust which comprises 3% by weight of the active ingredient of formula I.
VI. 20 parts by weight of an active compound of the formula I are mixed intimately with 2 parts by weight of calcium dodecylbenzenesulfonate, 8 parts by weight of fatty alcohol polyglycol ether, 2 parts by weight of the sodium salt of a phenol/urea/formaldehyde condensate and 68 parts by weight of a paraffinic mineral oil. This gives a stable oily dispersion.
VII. 1 part by weight of an active compound of the formula I is dissolved in a mixture composed of 70 parts by weight of cyclohexanone, 20 parts by weight of ethoxylated isooctylphenol and 10 parts by weight of ethoxylated castor oil. This gives a stable emulsion concentrate.
VIII. 1 part by weight of an active compound of the formula I is dissolved in a mixture composed of 80 parts by weight of cyclohexanone and 20 parts by weight of Wettole EM 31 (=nonionic emulsifier based on ethoxylated castor oil). This gives a stable emulsion concentrate.

The compounds of the formula I or the herbicidal compositions can be applied pre- or post-emergence. If the active ingredients are less well tolerated by certain crop plants, application techniques may be used in which the herbicidal compositions are sprayed, with the aid of the spraying equipment, in such a way that as far as possible they do not come into contact with the leaves of the sensitive crop plants, while the active ingredients reach the leaves of undesirable plants growing underneath, or the bare soil surface (post-directed, lay-by).

The rates of application of the compound of the formula I are from 0.001 to 3.0, preferably 0.01 to 1.0, kg/ha of active substance (a.s.), depending on the control target, the season, the target plants and the growth stage.

To widen the spectrum of action and to achieve synergistic effects, the benzoyl-substituted alanines of the formula I may be mixed with a large number of representatives of other herbicidal or growth-regulating active ingredient groups and then applied concomitantly. Suitable components for mixtures are, for example, 1,2,4-thiadiazoles, 1,3,4-thiadiazoles, amides, aminophosphoric acid and its derivatives, aminotriazoles, anilides, (het)aryloxyalkanoic acids and their derivatives, benzoic acid and its derivatives, benzothiadiazinones, 2-(het)aroyl-1,3-cyclohexanediones, hetaryl aryl ketones, benzylisoxazolidinones, meta-CF₃-phenyl derivatives, carbamates, quinolinecarboxylic acid and its derivatives, chloroacetanilides, cyclohexenone oxime ether derivatives, diazines, dichloropropionic acid and its derivatives, dihydro-benzofurans, dihydrofuran-3-ones, dinitroanilines, dinitrophenols, diphenyl ethers, dipyridyls, halocarboxylic acids and their derivatives, ureas, 3-phenyluracils, imidazoles, imidazolinones, N-phenyl-3,4,5,6-tetrahydrophthalimides, oxadiazoles, oxiranes, phenols, aryloxy- and hetaryloxyphenoxypropionic esters, phenylacetic acid and its derivatives, 2-phenylpropionic acid and its derivatives, pyrazoles, phenylpyrazoles, pyridazines, pyridinecarboxylic acid and its derivatives, pyrimidyl ethers, sulfonamides, sulfonylureas, triazines, triazinones, triazolinones, triazolecarboxamides and uracils.

It may furthermore be beneficial to apply the compounds of the formula I alone or in combination with other herbicides, or in the form of a mixture with other crop protection agents, for example together with agents for controlling pests or phytopathogenic fungi or bacteria. Also of interest is the miscibility with mineral salt solutions, which are employed for treating nutritional and trace element deficiencies. Non-phytotoxic oils and oil concentrates may also be added.

### USE EXAMPLES

The herbicidal activity of the benzoyl-substituted alanines of the formula I was demonstrated by the following greenhouse experiments:

The culture containers used were plastic flowerpots containing loamy sand with approximately 3.0% of humus as the substrate. The seeds of the test plants were sown separately for each species.

For the pre-emergence treatment, the active ingredients, which had been suspended or emulsified in water, were applied directly after sowing by means of finely distributing nozzles. The containers were irrigated gently to promote germination and growth and subsequently covered with transparent plastic hoods until the plants had rooted. This cover causes uniform germination of the test plants, unless this has been impaired by the active ingredients.

For the post-emergence treatment, the test plants were first grown to a height of 3 to 15 cm, depending on the plant habit, and only then treated with the active ingredients which had been suspended or emulsified in water. For this purpose, the test plants were either sown directly and grown in the same containers, or they were first grown separately as seedlings and transplanted into the test containers a few days prior to treatment. The rate of application for the post-emergence treatment was 0.5 or 1.0 kg/ha of a.s. (active substance).

Depending on the species, the plants were kept at 10-25° C. or 20-35° C. The test period extended over 2 to 4 weeks. During this time, the plants were tended, and their response to the individual treatments was evaluated.

Evaluation was carried out using a scale from 0 to 100. 100 means no emergence of the plants, or complete destruction of at least the aerial parts, and 0 means no damage, or normal course of growth.

The plants used in the greenhouse experiments belonged to the following species:

| Scientific name | Common Name |
|---|---|
| Amaranthus retroflexus | pig weed |
| Chenopodium album | lambsquarters |

At application rates of 0.5 kg/ha, the compound 3.27 (Table 3) showed very good post-emergence action against the unwanted plants Amaranthus retroflexus and Chenopodium album.

Furthermore, the compounds 3.18, 3.20, 3.21, 3.22, 3.24, 3.25, 3.26, 3.32 and 3.44 (Table 3), applied by the post-emergence method at application rates of 1.0 kg/ha, effected very good control of the harmful plants Amaranthus retroflexus and Chenopodium album.

1-11. (canceled)

12. A benzoyl-substituted alanine of the formula I 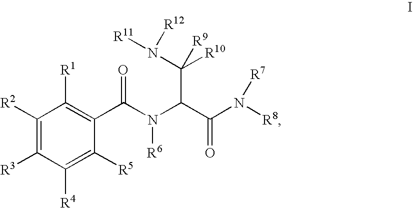
wherein the variables are as defined below:
R¹ is halogen, cyano, C₁-C₆-alkyl, C₁-C₆-haloalkyl or C₁-C₆-haloalkoxy;
R², R³, R⁴, R⁵ are hydrogen, halogen, cyano, C₁-C₆-alkyl, C₁-C₆-haloalkyl, C₁-C₆-alkoxy or C₁-C₆-haloalkoxy;
R⁶, R⁷ are hydrogen, hydroxy or C₁-C₆-alkoxy;
R⁸ is C₁-C₆-alkyl, C₁-C₄-cyanoalkyl or C₁-C₆-haloalkyl;
R⁹ is hydrogen or C₁-C₆-alkyl;
R¹⁰ is hydrogen, C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₁-C₆-cyanoalkyl, C₂-C₆-cyanoalkenyl, C₂-C₆-cyanoalkynyl, C₁-C₆-hydroxyalkyl, C₂-C₆-hydroxyalkenyl, C₂-C₆-hydroxyalkynyl, C₃-C₆-cycloalkyl, C₃-C₆-cycloalkenyl, 3- to 6-membered heterocyclyl,
wherein the cycloalkyl, cycloalkenyl or 3- to 6-membered heterocyclyl radicals mentioned above may be partially or fully halogenated and/or may carry one to three radicals from the group consisting of oxo, cyano, nitro, C₁-C₆-alkyl,
C₁-C₆-haloalkyl, hydroxy, C₁-C₆-alkoxy, C₁-C₆-haloalkoxy, hydroxycarbonyl, C₁-C₆-alkoxycarbonyl, hydroxycarbonyl-C₁-C₆-alkoxy, C₁-C₆-alkoxycarbonyl-C₁-C₆-alkoxy, amino, C₁-C₆-alkylamino, di(C₁-C₆-alkyl)amino, C₁-C₆-alkyl-sulfonylamino, C₁-C₆-haloalkylsulfonylamino, aminocarbonylamino, (C₁-C₆-alkylamino)carbonylamino, di(C₁-C₆-alkyl)aminocarbonylamino, aryl and aryl(C₁-C₆-alkyl);
C₁-C₆-alkoxy-C₁-C₄-alkyl, C₂-C₆-alkenyloxy-C₁-C₄-alkyl, C₂-C₆-alkynyloxy-C₁-C₄-alkyl, C₁-C₆-haloalkoxy-C₁-C₄-alkyl, C₂-C₆-haloalkenyloxy-C₁-C₄-alkyl, C₂-C₆-haloalkynyloxy-C₁-C₄-alkyl, C₁-C₆-alkoxy-C₁-C₄-alkoxy-C₁-C₄-alkyl, C₁-C₆-alkylthio-C₁-C₄-alkyl, C₂-C₆-alkenylthio-C₁-C₄-alkyl, C₂-C₆-alkynylthio-C₁-C₄-alkyl, C₁-C₆-haloalkyl-C₁-C₄-thioalkyl, C₂-C₆-haloalkenyl-C₁-C₄-thioalkyl,
C₂-C₆-haloalkynyl-C₁-C₄-thioalkyl, C₁-C₆-alkylsulfinyl-C₁-C₄-alkyl,
C₁-C₆-haloalkylsulfinyl-C₁-C₄-alkyl, C₁-C₆-alkylsulfonyl-C₁-C₄-alkyl,
C₁-C₆-haloalkylsulfonyl-C₁-C₄-alkyl, amino-C₁-C₄-alkyl, C₁-C₆-alkylamino-C₁-C₄-alkyl, di(C₁-C₆-alkyl)amino-C₁-C₄-alkyl, C₁-C₆-alkylsulfonylamino-C₁-C₄-alkyl, C₁-C₆-alkylsulfonyl(C₁-C₆-alkyl)amino-C₁-C₄-alkyl, C₁-C₆-alkylcarbonyl, hydroxycarbonyl, C₁-C₆-alkoxycarbonyl, aminocarbonyl, C₁-C₆-alkylaminocarbonyl, di(C₁-C₆-alkyl)aminocarbonyl, formylamino-C₁-C₄-alkyl, C₁-C₆-alkoxycarbonylamino-C₁-C₄-alkyl, C₁-C₆-alkylcarbonyl-C₁-C₆-alkyl, hydroxycarbonyl-C₁-C₄-alkyl, C₁-C₆-alkoxycarbonyl-C₁-C₄-alkyl, C₁-C₆-haloalkoxycarbonyl-C₁-C₄-alkyl, C₁-C₆-alkylcarbonyloxy-C₁-C₄-alkyl, aminocarbonyl-C₁-C₄-alkyl, C₁-C₆-alkylaminocarbonyl-C₁-C₄-alkyl, di(C₁-C₆-alkyl)aminocarbonyl-C₁-C₄-alkyl, C₁-C₆-alkylcarbonylamino-C₁-C₄-alkyl, C₁-C₆-alkylcarbonyl-(C₁-C₆-alkylamino)-C₁-C₄-alkyl, (C₁-C₆-alkyl)aminocarbonyloxy-C₁-C₄-alkyl, di(C₁-C₆-alkyl)aminocarbonyloxy-C₁-C₄-alkyl, [(C₁-C₆-alkyl)aminocarbonylamino]C₁-C₄-alkyl, [di(C₁-C₆-alkyl)aminocarbonylamino]C₁-C₄-alkyl; phenyl-C₁-C₄-alkyl, phenyl-C₂-C₄-alkenyl, phenyl-C₂-C₄-alkynyl, phenyl-C₁-C₄-haloalkyl, phenyl-C₂-C₄-haloalkenyl, phenyl-C₂-C₄-haloalkynyl, phenyl-C₁-C₄-hydroxyalkyl, phenyl-C₂-C₄₋hydroxyalkenyl, phenyl-C₂-C₄-hydroxyalkynyl, phenylcarbonyl-C₁-C₄-alkyl, phenylcarbonyloxy-C₁-C₄-alkyl, phenyloxycarbonyl-C₁-C₄-alkyl, phenyloxy-C₁-C₄-alkyl, phenylthio-C₁-C₄-alkyl, phenylsulfinyl-C₁-C₄-alkyl, phenylsulfonyl-C₁-C₄-alkyl, heteroaryl, heteroaryl-C₁-C₄-alkyl, heteroaryl-C₂-C₄-alkenyl, heteroaryl-C₂-C₄-alkynyl, heteroaryl-C₁-C₄-haloalkyl, heteroaryl-C₂-C₄-haloalkenyl, heteroaryl-C₂-C₄-haloalkynyl, heteroaryl-C₁-C₄-hydroxyalkyl, heteroaryl-C₂-C₄-hydroxyalkenyl, heteroaryl-C₂-C₄-hydroxyalkynyl, heteroarylcarbonyl-C₁-C₄-alkyl, heteroarylcarbonyloxy-C₁-C₄-alkyl, heteroaryloxycarbonyl-C₁-C₄-alkyl, heteroaryloxy-C₁-C₄-alkyl, heteroarylthio-C₁-C₄-alkyl, or heteroarylsulfinyl-C₁-C₄-alkyl, heteroarylsulfonyl-C₁-C₄-alkyl,
wherein the phenyl and heteroaryl radicals mentioned above may be partially or fully halogenated and/or may carry one to three radicals from the group consisting of cyano, nitro, C₁-C₆-alkyl, C₁-C₆-haloalkyl, hydroxy, C₁-C₆-hydroxyalkyl, C₁-C₆-alkoxy, C₁-C₆-haloalkoxy, hydroxycarbonyl, C₁-C₆-alkoxycarbonyl, hydroxycarbonyl-C₁-C₆-alkoxy, C₁-C₆-alkoxy-carbonyl-C₁-C₆-alkoxy, amino, C₁-C₆-alkylamino, di(C₁-C₆-alkyl)amino, C₁-C₆-alkylsulfonylamino, C₁-C₆-haloalkylsulfonylamino, (C₁-C₆-alkylamino)carbonylamino, di(C₁-C₆-alkyl)-aminocarbonylamino, aryl and aryl(C₁-C₆-alkyl);
R¹¹ is hydrogen, C₁-C₆-alkyl, C₃-C₆-cycloalkyl, C₃-C₆-alkenyl, C₃-C₆-alkynyl, C₃-C₆-haloalkenyl, C₃-C₆-haloalkynyl, formyl, C₁-C₆-alkylcarbonyl, C₃-C₆-cycloalkylcarbonyl, C₂-C₆-alkenylcarbonyl, C₂-C₆-alkynylcarbonyl, C₁-C₆-alkoxycarbonyl, C₃-C₆-alkenyloxycarbonyl, C₃-C₆-alkynyloxycarbonyl, aminocarbonyl, C₁-C₆-alkylaminocarbonyl, C₃-C₆-alkenylaminocarbonyl, C₃-C₆-alkynylaminocarbonyl, C₁-C₆-alkylsulfonylaminocarbonyl, di(C₁-C₆-alkyl)aminocarbonyl, N—(C₃-C₆-alkenyl)-N—(C₁-C₆-alkyl)aminocarbonyl, N—(C₃-C₆-alkynyl)-N—(C₁-C₆-alkyl)aminocarbonyl, N—(C₁-C₆-alkoxy)-N—(C₁-C₆-alkyl)aminocarbonyl, N—(C₃-C₆-alkenyl)-N—(C₁-C₆-alkoxy)aminocarbonyl, N—(C₃-C₆-alkynyl)-N—(C₁-C₆-alkoxy)aminocarbonyl, di(C₁-C₆-alkyl)aminothiocarbonyl, (C₁-C₆-alkyl)cyanoimino, (amino)cyanoimino, [(C₁-C₆-alkyl)amino]cyanoimino, di(C₁-C₆-alkyl)aminocyanoimino, C₁-C₆-alkylcarbonyl-C₁-C₆-alkyl, C₁-C₆-alkoxyimino-C₁-C₆-alkyl, N—(C₁-C₆-alkylamino)imino-C₁-C₆-alkyl, N-[di-(C₁-C₆-alkyl)amino]imino-C₁-C₆-alkyl or tri-C₁-C₄-alkylsilyl, wherein the alkyl, cycloalkyl and alkoxy radicals mentioned may be partially or fully halogenated and/or may carry one to three of the following groups: cyano, hydroxy, C₃-C₆-cycloalkyl, C₁-C₆-alkoxy-C₁-C₄-alkyl, C₁-C₄-alkoxy-C₁-C₄-alkoxy-C₁-C₄-alkyl, C₁-C₄-alkoxy, C₁-C₄-alkylthio, di(C₁-C₄-alkyl)amino, C₁-C₄-alkyl-C₁-C₆-alkoxycarbonylamino, C₁-C₄-alkylcarbonyl, hydroxycarbonyl, C₁-C₄-alkoxycarbonyl, aminocarbonyl, C₁-C₄-alkylaminocarbonyl, di(C₁-C₄-alkyl)aminocarbonyl or C₁-C₄-alkylcarbonyloxy;
phenyl, phenyl-C₁-C₆-alkyl, phenylcarbonyl-C₁-C₆-alkyl, phenoxycarbonyl, phenylaminocarbonyl, phenylsulfonylaminocarbonyl, N—(C₁-C₆-alkyl)-N-(phenyl)aminocarbonyl, or phenyl-C₁-C₆-alkylcarbonyl,
wherein the phenyl radical may be partially or fully halogenated and/or may carry one to three of the following groups: nitro, cyano, C₁-C₄-alkyl, C₁-C₄-haloalkyl, C₁-C₄-alkoxy or C₁-C₄-haloalkoxy; or
SO₂R¹³;
R¹² is hydrogen, C₁-C₆-alkyl, C₃-C₆-cycloalkyl, C₃-C₆-alkenyl, C₃-C₆-alkynyl, C₃-C₆-haloalkenyl, C₃-C₆-haloalkynyl, hydroxy or C₁-C₆-alkoxy;
R¹³ is C₁-C₆-alkyl, C₁-C₆-haloalkyl or phenyl,
wherein the phenyl radical may be partially or fully halogenated and/or may carry one to three of the following groups: C₁-C₆-alkyl, C₁-C₆-haloalkyl or C₁-C₆-alkoxy;
or an agriculturally useful salt thereof.

13. The benzoyl-substituted alanine of the formula I according to claim 12, wherein R¹ is halogen or C₁-C₆-haloalkyl.

14. The benzoyl-substituted alanine of the formula I according to claim 12, wherein R² and R³, independently of one another, are hydrogen, halogen or C₁-C₆-haloalkyl.

15. The benzoyl-substituted alanine of the formula I according to claim 12, wherein R⁴, R⁵, R⁶, R⁷, R⁹ and R¹² are hydrogen.

16. The benzoyl-substituted alanine of the formula I according to claim 12, wherein R¹⁰ is C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₁-C₆-cyanoalkyl, C₁-C₆-hydroxyalkyl, C₂-C₆-hydroxyalkenyl, C₂-C₆-hydroxyalkynyl, C₃-C₆-cycloalkyl, C₃-C₆-cycloalkenyl, 3- to 6-membered heterocyclyl,
wherein the cycloalkyl, cycloalkenyl or 3- to 6-membered heterocyclyl radicals mentioned above may be partially or fully halogenated and/or may carry one to three radicals from the group consisting of oxo, C₁-C₆-alkyl, C₁-C₆-haloalkyl, hydroxycarbonyl and C₁-C₆-alkoxycarbonyl,
C₁-C₆-alkoxy-C₁-C₄-alkyl, C₁-C₆-haloalkoxy-C₁-C₄-alkyl, C₁-C₆-alkoxy-C₁-C₄-alkoxy-C₁-C₄-alkyl, C₁-C₆-alkylthio-C₁-C₄-alkyl, C₁-C₆-alkylsulfonylamino-C₁-C₄-alkyl, hydroxycarbonyl, C₁-C₆-alkoxycarbonyl, hydroxycarbonyl-C₁-C₄-alkyl, C₁-C₆-alkoxycarbonyl-C₁-C₄-alkyl, C₁-C₆-haloalkoxycarbonyl-C₁-C₄-alkyl, C₁-C₆-alkylcarbonyloxy-C₁-C₄-alkyl, C₁-C₆-alkylcarbonylamino-C₁-C₄-alkyl, di(C₁-C₆-alkyl)carbonylamino-C₁-C₄-alkyl, [di(C₁-C₆-alkylamino)carbonyloxy]C₁-C₄-alkyl, {di[di(C₁-C₆-alkyl)amino]carbonyloxy}C₁-C₄-alkyl, phenyl-C₁-C₄-alkyl, phenyl-C₂-C₄-alkenyl, phenyl-C₂-C₄-alkynyl, phenyl-C₁-C₄-haloalkyl, phenyl-C₂-C₄-haloalkenyl, phenyl-C₁-C₄-hydroxyalkyl, phenyloxy-C₁-C₄-alkyl, phenylthio-C₁-C₄-alkyl, phenylsulfinyl-C₁-C₄-alkyl, phenylsulfonyl-C₁-C₄-alkyl,
heteroaryl, heteroaryl-C₁-C₄-alkyl, heteroaryl-C₁-C₄-hydroxyalkyl, heteroaryloxy-C₁-C₄-alkyl, heteroarylthio-C₁-C₄-alkyl, heteroarylsulfinyl-C₁-C₄-alkyl, or heteroarylsulfonyl-C₁-C₄-alkyl,
wherein the phenyl and heteroaryl radicals mentioned above may be partially or fully halogenated and/or may carry one to three radicals from the group consisting of cyano, nitro, C₁-C₆-alkyl, C₁-C₆-haloalkyl, hydroxy, C₁-C₆-alkoxy, C₁-C₆-haloalkoxy, hydroxycarbonyl, C₁-C₆-alkoxycarbonyl, hydroxycarbonyl-C₁-C₆-alkoxy, C₁-C₆-alkylsulfonylamino and C₁-C₆-haloalkylsulfonylamino.

17. A process for preparing a benzoyl-substituted alanine of the formula I according to claim 12, wherein an alanine derivative of the formula V 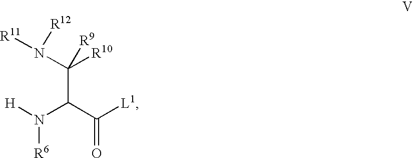
wherein R⁶, R⁹, R¹⁰, R¹¹ and R¹² are as defined in claim 1 and L¹ is hydroxy or C₁-C₆-alkoxy,
is reacted with a benzoic acid (derivative) of the formula IV 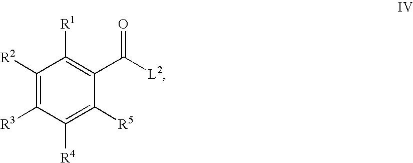
wherein R¹ to R⁵ are as defined in claim 12 and L² is hydroxy, halogen, C₁-C₆-alkylcarbonyl, C₁-C₆-alkoxycarbonyl, C₁-C₄-alkylsulfonyl, phosphoryl or iso-ureyl,
to give the corresponding benzoyl derivative of the formula III 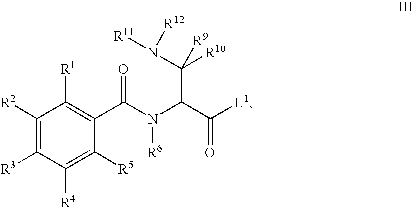
wherein R¹ to R⁶ and R⁹ to R¹² are as defined in claim 12 and L¹ is hydroxy or C₁-C₆-alkoxy,
and the resulting benzoyl derivative of the formula III is then reacted with an amine of the formula II
HNR⁷R⁸ II,
wherein R⁷ and R⁸ are as defined in claim 12,
to give the benzoyl-substituted alanine of formula I.

18. A benzoyl derivative of the formula III 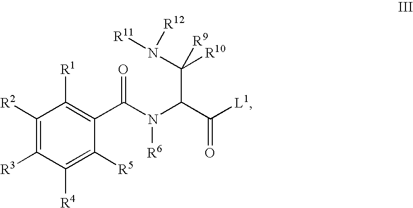
wherein R¹ to R⁶ and R⁹ to R¹² are as defined in claim 12 and L¹ is hydroxy or C₁-C₆-alkoxy.

19. A composition, comprising a herbicidally effective amount of at least one benzoyl-substituted alanine of the formula I or an agriculturally useful salt of formula I according to claim 12 and auxiliaries customary for formulating crop protection agents.

20. A process for preparing compositions comprising a herbicidally effective amount of at least one benzoyl-substituted alanine of the formula I or an agriculturally useful salt of formula I according to claim 12 and auxiliaries customary for formulating crop protection agents, wherein a herbicidally effective amount of at least one benzoyl-substituted alanine of the formula I or an agriculturally useful salt of formula I according to claim 12 and auxiliaries customary for formulating crop protection agents are mixed.

21. A method for controlling unwanted vegetation, wherein a herbicidally effective amount of at least one benzoyl-substituted alanine of the formula I or an agriculturally useful salt of formula I according to claim 12 is allowed to act on plants, their habitat and/or on seed.
